(19) United States
(12) Patent Application Publication
(10) Pub. No.: US 2009/0078384 A1
(43) Pub. Date: Mar. 26, 2009

(54) Esterases from rumen

(76) Inventors: Manuel Ferrer, Madrid (ES); Peter Golyshin, Wolfenbuttel (DE); Olga Golyshina, Wolfenbuttel (DE); Tatyana Chernikova, Braunschweig (DE); Carsten Strompl, Evessen (DE); Kenneth Timmis, Wolfenbuttel (DE); Kieran Elborough, Franklin (NZ); Graeme Jarvis, Wellington (NZ)

Correspondence Address: KLAUBER & JACKSON, 411 HACKENSACK AVENUE, HACKENSACK, NJ, 07601, US

(21) Appl. No.: 11/631,708

(22) PCT Filed: Jul. 6, 2005
(86) PCT No.: PCT/EP2005/007305

§ 371 Date: Aug. 21, 2008

(30) Foreign Application Priority Data
Jul. 6, 2004 (EP) 04015920.4

(51) Int. Cl. C12P 21/04; C12N 9/16; C12N 15/11; C07H 1/00; D21H 17/22; C11D 3/386; C12S 11/00; A23L 1/48; D21H 17/24; A23L 1/00; C12N 15/00; C12N 1/21

(52) U.S. Cl. 162/174; 435/196; 536/23.2; 435/320.1; 435/252.33; 435/69.1; 435/274; 426/61; 162/175; 510/392

(57) ABSTRACT

The invention provides polypeptides coding for new esterases from rumen. The invention also relates to functional fragments or functional derivatives thereof as well as to nucleic acids encoding the polypeptides of the invention, vectors and host cells containing said nucleic acids, a method for producing the polypeptides and the use of the polypeptides according to the present invention for various industrial purposes and medical treatments. The invention also relate to the conversion of said esterases into lipases.

The first invention relates to new esterases from rumen, in particular to polypeptides comprising or consisting of an amino acid sequence according to FIGS. 22 to 33 of the invention or a functional fragment or functional derivative thereof. The second invention herein relates to lipases, which are obtained by the conversion of said esterases.

Esterases are enzymes which are involved in degradation of plant cell walls and certain other substrates. Plant cell walls are composed mainly of cellulose, hemicellulose, xylan, lignin and xylo-oligomers. Xylan is the major constituent of hemicellulose and after cellulose it is the most abundant renewable polysaccharide in nature. It is located predominantly in the secondary cell walls of angiosperms and gymnosperms. The composition and structure of xylan are more complicated than that of cellulose and can vary quantitatively and qualitatively in various woody plant species, grasses and cereals. Xylan is a heteropolymer in which the constituents are linked together not only by glycosidic linkages but also by ester linkages. In detail, xylan of hemicellulosic polysaccharide plant cell walls is predominantly a 1,4-β-d-xylose polymer and is commonly substituted to various degrees with acetyl, arabinosyl and glucuronyl residues (Whistler, R. L. & Richards, E. L. (1970), The Carbohydrates—Chemistry and Biochemistry, pp. 447-469, Edited by W. Pigman & D. Horton. New York: Academic Press). About 60-70% of xylose residues are esterified at the hydroxyl group with acetic acid.

The structural complexity of xylan requires the cooperation of xylanases and b-xylosidases with several accessory enzymes for its biodegradation. Several bacteria and fungi grow on xylan as a carbon source by using an array of enzymes, such as endoxylanases and β-xylosidases.

One class of enzymes (showing hydrolytic activity) incorporated in the hydrolysis of Xylan are hydrolases (EC 3.2.1) involved in the hydrolysis of the glycosidic bonds of xylan. Another class of enzymes (showing esterolytic activity) incorporated in the hydrolysis of Xylan are esterases that hydrolyze the ester linkages.

In summary, beside a number of fibrolytic enzymes, which are needed to degrade components of plant cell walls as hemicellulose, xylases, β-xylanases, arabinofuranosidase, cellulases, glucanohydrolases, glucosidases and endoglucanases, esterases are also important enzymes influencing the digestibility of plant cell-wall material by hydrolyzing acetyl groups at O-2 and/or O-3 of xylose. This deacetylation process increases biodegradability and renders cellulose more accessible for the attack of other polysaccharide hydrolytic enzymes (Grohmann K. et al. (1989), Appl. Biochem. Biotechnol. 20/21: 45-61).

Esterases are mainly used for industrial purposes. Such industrial uses include, for example, the use in cosmetics, pulp and paper industry, feed processing, detergents or detergent compositions, synthesis of carbohydrate derivatives, such as sugar derivatives, or as food additive, e.g. flavour enhancer. Moreover, esterases are useful as research reagents in studies on plant cell wall structure, particularly the nature of covalent bonds between lignin and carbohydrate polymers in the cell wall matrix, and in studies on mechanisms related to disease resistance in plants and the process of organic matter decomposition. Furthermore, esterases are useful in selection of plants bred for production of highly digestible animal feeds, particularly for ruminant animals.

For industrial application esterase are desired, which show high stability and high substrate specifity, activity at optimal pH and optimal temperature, low addition of cations, high enantioselectivity and resistance towards detergents and solvents.

Although some of the investigated esterases in the art may fulfill the essential requirements upon which various industrial processes are based, there is a high need for new esterases with improved properties in stability, substrate specifity, enzyme rate, pH tolerance, temperature stability, low addition of cations, enantioselectivity and resistance towards detergents and solvents.

Despite vast information available in the art about numerous esterases having desirable properties for certain applications, esterases or esterase compositions, which simultaneously exhibit some or preferable several most of the aforementioned properties are not known.

Therefore, it is an object of the present invention to provide esterases with improved properties, preferably a combination of improved properties, which are useful for industrial applications.

The first invention of this application provides polypeptides comprising an amino acid sequence of amino acids No. 90 to No. 120 of one of the amino acid sequences shown in FIGS. 22 to 33 or a functional fragment, or functional derivative thereof.

Preferably, the polypeptide of the invention comprises an amino acid sequence of amino acids No. 90 to No. 120, preferably No. 85 to No. 135, more preferably No. 70 to No. 160 most preferably No. 60 to No. 175 of one of the amino acid sequences shown in FIGS. 22 to 33.

In an preferred embodiment the polypeptide of the invention comprises one of the amino acid sequences shown in FIGS. 22 to 33.

The invention is based on the discovery that symbiotic rumen ecosystem consists of mostly obligate anaerobic microorganisms including fungi, protozoa, bacteria and archaea. Thus, rumen ecosystems represent a unique microbial ecosystem with a high potential of microbial and manifold enzymatic diversity including hemicellulose, xylases, β-xylanases, arabinofuranosidase, cellulases, glucanohydrolases, glucosidases, endoglucanases as well as esterases, especially phenolic acid esterases and acetylxylan esterases. Consequently, according to a preferred embodiment of the invention, the polypeptide is derived from rumen, particularly from rumen ecosystem, preferably from cow rumen, more preferably from New Zealand dairy cow.

According to the invention an expression library from DNA extracted from rumen ecosystem was created and analysed by an activity selection technique, using alpha-naphthyl acetate as esterase substrate and Fast Blue RR. This expression library from DNA was screened for esterase activity. Individual proteins were expressed and genes featuring esterase phenotype were selected, purified in analytical scale and preliminary characterized in terms of stability, activity, regio- and stereospecificity, thermostability, salinity, solvent resistance, etc.

In general, the esterases of the invention relates to carboxylic acid esterases comprising several subclasses, e.g., feruloyl esterases and carbohydrate esterases.

Feruloyl esterases involved e.g., in breaking down the bond between the arabinase and ferulic acid, releasing the covalently bound lignin from hemicelluloses, were detected. These include clones pBKR. 13, pBKR.17, pBKR.35, pBKR.41, pBKR.43, pBKR.44 and pBKR.45). Thus, a preferred embodiment relates to a polypeptide, which releases covalently bound lignin from hemicelluloses. Consequently, another preferred embodiment of the invention relates to polypeptide, which represents a feruloyl esterase.

Carbohydrate esterases, that release acetic acid from carbohydrate (xylose, glucose or cellulose) and acetate esters, have also been detected. These include clones pBKR.13, pBKR.17, pBKR.34, pBKR.35, pBKR.41, pBKR.43, pBKR.44 and pBKR.45). Thus, a preferred embodiment relates to a polypeptide, which releases acetic acid from carbohydrates. Consequently, another preferred embodiment of the invention relates to polypeptide, which represents a carbohydrate esterase.

Several esterases were identified, which show esterolytic activities towards p-nitrophenyl esters and p-nitrophenyl acetates (in particular esterase clones pBKR.9, pBKR.14, pBKR.27, and pBKR.40). Consequently, a preferred embodiment of the invention relates to a polypeptide hydrolyzing p-nitrophenyl acetates and/or p-nitrophenyl esters, preferably p-nitrophenyl esters containing from 2 to 12 carbon atoms. This esterases cannot release ferulate or acetyl groups attached to xylose or xylan polymers or between arabinosyl groups and phenolic moieties such as ferulic acid (feruloyl esterase, EC 3.1.1.73) and p-coumaric acid (coumaroyl esterase).

Furthermore, several esterases pBKR.32 and pBKR.45) were identified, which release acetate from acetylated substrates, i.e., they showed acetylxylan esterase activity. Consequently, a preferred embodiment of the invention relates to a polypeptide releasing acetate from acetylated substrates. Another preferred embodiment relates to a polypeptide, which represents an acetylxylan esterase.

Several esterases, namely clones pBKR. 13, 27, 40, 41 and 43, seems to belong to a new family of esterases (see also illustration to FIGS. 11 to 34 below).

High performance esterases (enzymes showing esterolytic activity), which hydrolyze i.a. p-nitrophenyl esters, p-nitrophenyl acetates, carboxylic acids, primary or secondary alcohols, lactones and acetylated substrates were defined. Most of the polypeptides of the invention show esterolytic activity, which is considerably higher than of esterases known in the art (see i.a. FIGS. 1, 9), are highly stable towards tested detergents and solvents, highly stable over a broad pH ranging from pH 7.5 to pH 12.0 and at a temperature of up to 60° C. and/or are not influenced by mono- and divalent cations (see FIGS. 2 to 5). Moreover, they show high enantiomeric ratio towards esters of chiral carboxylic acids and chiral esters of primary and secondary alcohols (see FIGS. 6 to 8).

Consequently, in preferred embodiments of the invention functional active polypeptides show activity
(i) at pH optimum, preferably at pH ranging from 7.5 to 12.0, more preferably from 7.5 to 8.5 or from 9.5 to 10.0 or from 11.0 to 12.0,
(ii) at temperature optimum, preferably at a temperature from 40° C. to 60° C., more preferably from 40° C. to 50° C. or from 50° C. to 60° C. and/or
(iii) at low addition of cations, preferably without any addition of cations.

Furthermore, preferred embodiments relate to polypeptides showing
(i) highly specific activities and/or
(ii) high stability towards its substrate and/or
(iii) high enantioselectivity towards its substrate.

Some or all of these features may be realized by functional active polypeptides of the invention. In a particularly preferred embodiment of the invention, the polypeptide shows a combination of at least two, preferably three, more preferably four, even more preferably five, most preferably all six of the aforementioned features.

Activity, i.e. hydrolyzing activity of the polypeptide of the invention, at a “pH optimum” means that the polypeptide shows activity and is stable towards its substrate at a pH, which is optimal for the individual application.

Correspondingly, activity at a “temperature optimum” depends on specific use of the functional active polypeptides of the invention and means that the polypeptides show activity and are stable towards their environment conditions at a temperature, which is optimal for the respective application. Usually, a temperature stability is required from 40° C. up to 60° C. for the functional active polypeptides of the invention. For most biotechnical applications a temperature stability of the functional active polypeptide according to the invention at 60° C. is preferred.

Most enzymes, particularly enzymes showing esterolytic activity, require the presence of cations for their activity. Usually, mono- or divalent cations as NH₄⁺, K⁺, Li⁺, Na⁺ or Ca²⁺, Mn²⁺, Mg²⁺, Sr²⁺, Fe²⁺, Cu²⁺, Ni²⁺, Co²⁺, Zn²⁺ have to be added to obtain satisfying enzymatic activity. Thus, “activity at low addition of cations”, preferably activity without addition of cations, means that a polypeptide of the invention shows activity and is stable towards its environment conditions at a low cation concentration, preferably without addition of any cations at all.

“High specific activity” of the polypeptide of the invention means that its activity is essentially directed only towards its substrates.

“High enantioselectivity” and “enantiomeric ratio (E)” of the polypeptide of the invention means preferential selection of one enantiomer over another. A simple program to calculate the enantiomeric ratio is e.g., freely available at http://www.orgc.tugraz.ac.at. A nonselective reaction has an E value of 1, while resolutions with E>20 are considered good for synthesis.

A preferred embodiment relates to esterases of the invention, which are converted into lipases. Despite their relationship in sequence and structure, these enzymes differ in their profile for chain length specificity. Whereas esterases (EC 3.1.1.1) preferentially hydrolyze water-soluble esters and usually only triglycerides composed of short-chain fatty acids, especially shorter than C6, lipases (EC 3.1.1.3) prefer water-insoluble substrates, typically triglycerides composed of long-chain fatty acid (U. T. Bornscheuer, FEMS Microbiol. Rev. 2002, 26, 73-81).

“Functional”, e.g., functional fragment or functional derivative according to the invention means that the polypeptides exhibit esterolytic activity, particularly any esterolytic effect on esterase substrates. For example, it relates to a deacetylation process, i.e. hydrolysis of acetyl groups at O-2 and/or O-3 of xylose. Several methods for measuring esterolytic activity are known by a person skilled in the art (e.g., enzyme assays using marked substrates, substrate analysis by chromatographic methods (as HPLC or TLC) for separating enzyme and substrate and spectrophotometric assays for measuring esterolytic activity) (see e.g., Maniatis et al. (2001) Molecular Cloning: A laboratory manual, Cold Spring Harbor Laboratory Press, Cold Spring Harbor, N.Y.). Among various alternatives, an appropriate method is, for example, the esterase activity assay, as described below (see Example 4). This assay system is based on an activity selection technique with alpha-naphthyl acetate as assay substrate and Fast Blue RR.

The term “fragment of a polypeptide” according to the invention is intended to encompass a portion of a amino sequence disclosed herein of at least about 60 contiguous amino acids, preferably of at least about 80 contiguous amino acids, more preferably of at least about 100 contiguous amino acids or longer in length. Functional fragments of polypeptides that retain their esterolytic activity are particularly useful.

A “derivative of a polypeptide” according to the invention is intended to indicate a polypeptide, which is derived from the native polypeptide by substitution of one or more amino acids at one or two or more of different sites of the native amino acid sequence, deletion of one or more amino acids at either or both ends of the native amino acid sequence or at one or more sites of the amino acid sequence, or insertion of one or more amino acids at one or more sites of the native amino acid sequence retaining its characteristic activity, particularly esterolytic activity. Such a polypeptide can possess altered properties, which may be advantageous over the properties of the native sequence for certain applications (e.g. increased pH optimum, increased temperature stability etc.).

A derivative of a polypeptide according to the invention means a polypeptide, which has substantial identity with the amino acid sequences disclosed herein. Particularly preferred are nucleic acid sequences, which have at least 60% sequence identity, preferably at least 75% sequence identity, even more preferably at least 80%, yet more preferably 90% sequence identity and most preferably at least 95% sequence identity thereto. Appropriate methods for isolation of a functional derivative of a polypeptide as well as for determination of percent identity of two amino acid sequences are described below.

The production of such polypeptide fragments or derivatives (as described below) is well known and can be carried out following standard methods, which are well known by a person skilled in the art (see e.g., Maniatis et al. (2001) supra). In general, the preparation of such functional fragments or derivatives of a polypeptide can be achieved by modifying a DNA sequence, which encode the native polypeptide, transformation of that DNA sequence into a suitable host and expression of the modified DNA sequence to form the functional derivative of the polypeptide with the provision that the modification of the DNA does not disturb the characteristic activity, particularly esterolytic activity.

The isolation of these polypeptide fragments or derivatives can be carried out using standard methods as e.g. separation from cell or culture medium by centrifugation, filtration or chromatography and precipitation procedures (see, e.g., Maniatis et al. (2001) supra).

The polypeptide of the invention can also be fused to at least one second moiety. Preferably, the second or further moiety/moieties does not occur in the esterase as found in nature. The at least second moiety can be an amino acid, oligopeptide or polypeptide and can be linked to the polypeptide of the invention at a suitable position, for example, the N-terminus, the C-terminus or internally. Linker sequences can be used to fuse the polypeptide of the invention with at least one other moiety/moieties. According to one embodiment of the invention, the linker sequences preferably form a flexible sequence of 5 to 50 residues, more preferably 5 to 15 residues. In a preferred embodiment the linker sequence contains at least 20%, more preferably at least 40% and even more preferably at least 50% Gly residues. Appropriate linker sequences can be easily selected and prepared by a person skilled in the art. Additional moieties may be linked to the inventive sequence, if desired. If the polypeptide is produced as a fusion protein, the fusion partner (e.g., HA, HSV-Tag, His6) can be used to facilitate purification and/or isolation. If desired, the fusion partner can then be removed from polypeptide of the invention (e.g., by proteolytic cleavage or other methods known in the art) at the end of the production process.

According to another embodiment of the invention a nucleic acid encoding a polypeptide of the invention (or a functional fragment or functional derivative thereof) or a functional fragment or functional derivative of said nucleic acid is provided. Preferably, the nucleic acid comprises or consists of one of the nucleic acid sequences of FIGS. 10 to 21.

The nucleic acids of the invention can be DNA or RNA, for example, mRNA. The nucleic acid molecules can be double-stranded or single-stranded; single stranded RNA or DNA can be either the coding (sense) strand or the non-coding (antisense) strand. If desired, the nucleotide sequence of the isolated nucleic acid can include additional non-coding sequences such as non-coding 3′- and 5′-sequences (including regulatory sequences, for example). All nucleic acid sequences, unless otherwise designated, are written in the direction from the 5′ end to the 3′ end.

Furthermore, the nucleic acids of the invention can be fused to a nucleic acid comprising, for example, a marker sequence or a nucleotide sequence, which encodes a polypeptide to assist, e.g., in isolation or purification of the polypeptide. Representative sequences include, but are not limited to those, which encode a glutathione-S-transferase (GST) fusion protein, a polyhistidine (e.g., His6), hemagglutinin, HSV-Tag, for example.

The term “nucleic acid” also relates to a fragment or derivative of said nucleic acid as described below.

The term “fragment of a nucleic acid” is intended to encompass a portion of a nucleotide sequence described herein, which is from at least about 25 contiguous nucleotides to at least about 50 contiguous nucleotides, preferably at least about 60 contiguous nucleotides, more preferably at least about 120 contiguous nucleotides, most preferably at least about 180 contiguous nucleotides or longer in length. Especially, shorter fragments according to the invention are useful as probes and also as primers. Particularly preferred primers and probes selectively hybridize to the nucleic acid molecule encoding the polypeptides described herein. A primer is a nucleic acid fragment, which functions as an initiating substrate for enzymatic or synthetic elongation. A probe is a nucleic acid sequence, which hybridizes with a nucleic acid sequence of the invention, a fragment or a complementary nucleic acid sequence thereof. Fragments, which encode polypeptides according to the invention that retain activity are particularly useful.

Hybridization can be used herein to analyze whether a given fragment or gene corresponds to the esterases described herein and thus falls within the scope of the present invention. Hybridization describes a process in which a strand of nucleic acid joins with a complementary strand through base pairing. The conditions employed in the hybridization of two non-identical, but very similar, complementary nucleic acids varies with the degree of complementary of the two strands and the length of the strands. Such conditions and hybridisation techniques are well known by a person skilled in the art and can be carried out following standard hybridization assays (see e.g., Maniatis et al. (2001) supra). Consequently, all nucleic acid sequences, which hybridize to the nucleic acid or the functional fragments or functional derivatives thereof according to the invention are encompassed by the invention.

A “derivative of a nucleic acid” according to the invention is intended to indicate a nucleic acid, which is derived from the native nucleic acid corresponding to the description above relating to a “functional derivative of a polypeptide”, i.e. by addition, substitution, deletion or insertion of one or more nucleic acids retaining the characteristic activity, particularly esterolytid activity of said nucleic acid. Such a nucleic acid can exhibit altered properties in some specific aspect (e.g. increased or decreased expression rate).

Skilled artisans will recognize that the amino acids of polypeptides of the invention can be encoded by a multitude of different nucleic acid triplets because most of the amino acids are encoded by more than one nucleic acid triplet due to the degeneracy of the amino acid code. Because these alternative nucleic acid sequences would encode the same amino acid sequences, the present invention further comprises these alternate nucleic acid sequences.

A derivative of a nucleic acid according to the invention means a nucleic acid or a fragment or a derivative thereof, which has substantial identity with the nucleic acid sequences described herein. Particularly preferred are nucleic acid sequences, which have at least about 30%, preferably at least about 40%, more preferably at least about 50%, even more preferably at least about 60%, yet more preferably at least about 80%, still more preferably at least about 90%, and even more preferably at least about 95% identity with nucleotide sequences described herein.

To determine the percent identity of two nucleotide sequences, the sequences can be aligned for optimal comparison purposes (e.g., gaps can be introduced in the sequence of a first nucleotide sequence). The nucleotides at corresponding nucleotide positions can then be compared. When a position in the first sequence is occupied by the same nucleotide as the corresponding position in the second sequence, then the molecules are identical at that position. The percent identity between the two sequences is a function of the number of identical positions shared by the sequences.

The determination of percent identity of two sequences can be accomplished using a mathematical algorithm. A preferred, non-limiting example of a mathematical algorithm utilized for the comparison of two sequences is the algorithm of Karlin et al. (1993), PNAS USA, 90:5873-5877. Such an algorithm is incorporated into the NBLAST program, which can be used to identify sequences having the desired identity to nucleotide sequences of the invention. To obtain gapped alignments for comparison purposes, Gapped BLAST can be utilized as described in Altschul et al. (1997), Nucleic Acids Res, 25:3389-3402. When utilizing BLAST and Gapped BLAST programs, the default parameters of the respective programs (e.g., NBLAST) can be used. The described method of determination of the percent identity of two can be also applied to amino acid sequences.

The production of such nucleic acid fragments or derivatives (as described below) is well known and can be carried out following standard methods, which are well known by a person skilled in the art (see e.g., Maniatis et al. (2001) supra). In general, the preparation of such functional fragments or derivatives of a nucleic add can be achieved by modifying (altering) a DNA sequence, which encodes the native polypeptide and amplifying the DNA sequence with suitable means, e.g., by PCR technique. These mutations of the nucleic acids may be generated by either random mutagenesis techniques, such as those techniques employing chemical mutagens, or by site-specific mutagenesis employing oligonucleotides. These nucleic acids conferring substantially the same function, as described above, in substantially the same manner as the exemplified nucleic acids are also encompassed within the present invention.

Accordingly, derivatives of a polypeptide according to the invention (as described above) encoded by the nucleic adds of the invention may also be induced by alterations of the nucleic acids, which encodes these proteins.

One of the most widely employed technique for altering a nucleic acid sequence is by way of oligonucleotide-directed site-specific mutagenesis (see Comack B, CURRENT PROTOCOLS IN MOLECULAR BIOLOGY, 8.01-8.5.9, Ausubel F, et al., eds. 1991). In this technique an oligonucleotide, whose sequence contains a mutation of interest, is synthesized as described supra. This oligonucleotide is then hybridized to a template containing the wild-type nucleic acid sequence. In a preferred embodiment of this technique, the template is a single-stranded template. Particularly preferred are plasmids, which contain regions such as the f1 intergenic region. This region allows the generation of single-stranded templates when a helper phage is added to the culture harboring the phagemid. After annealing of the oligonucleotide to the template, a DNA-dependent DNA polymerase is used to synthesize the second strand from the oligonucleotide, complementary to the template DNA. The resulting product is a heteroduplex molecule containing a mismatch due to the mutation in the oligonucleotide. After DNA replication by the host cell a mixture of two types of plasmid are present, the wild-type and the newly constructed mutant. This technique permits the introduction of convenient restriction sites such that the coding nucleic acid sequence may be placed immediately adjacent to whichever transcriptional or translational regulatory elements are employed by the practitioner.

The construction protocols utilized for E. coli can be followed to construct analogous vectors for other organisms, merely by substituting, if necessary, the appropriate regulatory elements using techniques well known to skilled artisans.

The isolation of such nucleic acid functional fragments or functional derivatives (as described below) can be carried out by using standard methods as screening methods (e.g., screening of a genomic DNA library) followed by sequencing or hybridisation (with a suitable probe, e.g., derived by generating an oligonucleotide of desired sequence of the “target” nucleic acid) and purification procedures, if appropriate.

The invention also relates to isolated nucleic acids. An “isolated” nucleic acid molecule or nucleotide sequence is intended to mean a nucleic acid molecule or nucleotide sequence, which is not flanked by nucleotide sequences, which normally flank the gene or nucleotide sequence (as in genomic sequences) and/or has been completely or partially purified from other nucleic acids (e.g., as in an DNA or RNA library). For example, an isolated nucleic acid of the invention may be substantially isolated with respect to the complex cellular milieu in which it naturally occurs. In some instances, the isolated material will form a part of a composition (for example, a crude extract containing other substances), buffer system or reagent mix. In other circumstance, the material may be purified to essential homogeneity, for example as determined by PAGE or column chromatography such as HPLC. This meaning refers correspondingly to an isolated amino acid sequence.

The present invention also encompasses gene products of the nucleic acids of the invention coding for a polypeptide of the invention or a functional fragment or functional derivative thereof. Preferably the gene product codes for a polypeptide according to one of the amino acid sequences of FIGS. 22 to 33. Also included are alleles, derivatives or fragments of such gene products.

“Gene product” according to the invention relates not only to the transcripts, accordingly RNA, preferably mRNA, but also to polypeptides or proteins, particularly, in purified form.

“Derivatives” or “fragments” of a gene product are defined corresponding to the definitions or derivatives or fragments of the polypeptide or nucleic acid according to the invention.

The invention also provides a vector comprising the nucleic acid of the invention. The terms “construct”, “recombinant construct” and “vector” are intended to have the same meaning and define a nucleotide sequence, which comprises beside other sequences one or more nucleic acid sequences (or functional fragments, functional derivatives thereof) of the invention. A vector can be used, upon transformation into an appropriate host cell, to cause expression of the nucleic acid. The vector may be a plasmid, a phage particle or simply a potential genomic insert. Once transformed into a suitable host, the vector may replicate and function independently of the host genome, or may, under suitable conditions, integrate into the genome itself. Preferred vectors according to the invention are E. coli XL-Blue MRF′ and pBK-CMV plasmid.

The aforementioned term “other sequences” of a vector relates to the following: In general, a suitable vector includes an origin of replication, for example, Ori p, colEl Ori, sequences, which allow the inserted nucleic acid to be expressed (transcribed and/or translated) and/or a selectable genetic marker including, e.g., a gene coding for a fluorescence protein, like GFP, genes, which confer resistance to antibiotics such as the p-lactamase gene from Tn3, the kanamycin-resistance gene from Tn903 or the chloramphenicol-resistance gene from Tn9.

The term “plasmid” means an extrachromosomal usually self-replating genetic element. Plasmids are generally designated by a lower “p” preceded and/or followed by letters and numbers. The starting plasmids herein are either commercially available, publicly available on an unrestricted basis or can be constructed from available plasmids in accordance with the published procedures. In addition, equivalent plasmids to those described are known to a person skilled in the art. The starting plasmid employed to prepare a vector of the present invention may be isolated, for example, from the appropriate E. coli containing these plasmids using standard procedures such as cesium chloride DNA isolation.

A vector according to the invention also relates to a (recombinant) DNA cloning vector as well as to a (recombinant) expression vector. A DNA cloning vector refers to an autonomously replicating agent, including, but not limited to, plasmids and phages, comprising a DNA molecule to which one or more additional nucleic acids of the invention have been added. An expression vector relates to any DNA cloning vector recombinant construct comprising a nucleic acid sequence of the invention operably linked to a suitable control sequence capable of effecting the expression and to control the transcription of the inserted nucleic acid of the invention in a suitable host. Also, the plasmids of the present invention may be readily modified to construct expression vectors that produce the polypeptides of the invention in a variety of organisms, including, for example, E. coli, Sf9 (as host for baculovirus), Spodoptera and Saccharomyces. The literature contains techniques for constructing AV12 expression vectors and for transforming AV12 host cells. U.S. Pat. No. 4,992,373, herein incorporated by reference, is one of many references describing these techniques.

“Operably linked” means that the nucleic acid sequence is linked to a control sequence in a manner, which allows expression (e.g., transcription and/or translation) of the nucleic acid sequence.

“Transcription” means the process whereby information contained in a nucleic acid sequence of DNA is transferred to complementary RNA sequence

“Control sequences” are well known in the art and are selected to express the nucleic acid of the invention and to control the transcription. Such control sequences include, but are not limited to a polyadenylation signal, a promoter (e.g., natural or synthetic promoter) or an enhancer to effect transcription, an optional operator sequence to control transcription, a locus control region or a silencer to allow a tissue-specific transcription, a sequence encoding suitable ribosome-binding sites on the mRNA, a sequence capable to stabilize the mRNA and sequences that control termination of transcription and translation. These control sequences can be modified, e.g., by deletion, addition, insertion or substitution of one or more nucleic acids, whereas saving their control function. Other suitable control sequences are well known in the art and are described, for example, in Goeddel (1990), Gene Expression Technology: Methods in Enzymology 185, Academic Press, San Diego, Calif.

Especially a high number of different promoters for different organism is known. For example, a preferred promoter for vectors used in Bacillus subtilis is the AprE promoter; a preferred promoter used in E. coli is the T7/Lac promoter, a preferred promoter used in Saccharomyces cerevisiae is PGK1, a preferred promoter used in Aspergillus niger is glaA, and a preferred promoter used in Trichoderma reesei (reesei) is cbhI. Promoters suitable for use with prokaryotic hosts also include the beta-lactamase (vector pGX2907 (ATCC 39344) containing the replicon and beta-lactamase gene) and lactose promoter systems (Chang et al. (1978), Nature (London), 275:615; Goeddel et al. (1979), Nature (London), 281:544), alkaline phosphatase, the tryptophan (trp) promoter system (vector pATH1 (ATCC 37695) designed to facilitate expression of an open reading frame as a trpE fusion protein under control of the trp promoter) and hybrid promoters such as the tac promoter (isolatable from plasmid pDR540 ATCC-37282). However, other functional bacterial promoters, whose nucleotide sequences are generally known, enable a person skilled in the art to ligate them to DNA encoding the polypeptides of the instant invention using linkers or adapters to supply any required restriction sites. Promoters for use in bacterial systems also will contain a Shine-Delgarno sequence operably linked to the DNA encoding the desired polypeptides.

Useful expression vectors, for example, may consist of segments of chromosomal, non-chromosomal and synthetic DNA sequences such as various known derivatives of SV40 and known bacterial plasmids, e.g., plasmids from E. coli including col E1, pBK, pCR1, pBR322, pMb9, pUC 19 and their derivatives, wider host range plasmids, e.g., RP4, phage DNAs e.g., the numerous derivatives of phage lambda, e.g., NM989, and other DNA phages, e.g., M13 and filamentous single stranded DNA phages, yeast plasmids, vectors useful in eukaryotic cells, such as vectors useful in animal cells and vectors derived from combinations of plasmids and phage DNAs, such as plasmids, which have been modified to employ phage DNA or other expression control sequences. Expression techniques using the expression vectors of the present invention are known in the art and are described generally in, for example, Maniatis et al. (2001) supra.

The invention also provides a host cell comprising a vector or a nucleic acid (or a functional fragment, or a functional derivative thereof according to the invention.

“Host cell” means a cell, which has the capacity to act as a host and expression vehicle for a nucleic acid or a vector according to the present invention. The host cell can be e.g., a prokaryotic, an eukaryotic or an archaeon cell. Host cells comprising (for example, as a result of transformation, transfection or transduction) a vector or nucleic acid as described herein include, but are not limited to, bacterial cells (e.g., R. marinus, E. coli, Streptomyces, Pseudomonas, Bacillus, Serratia marcescens, Salmonella typhimurium), fungi including yeasts (e.g., Saccharomyces cerevisiae, Pichia pastoris) and molds (e.g., Aspergillus sp.), insect cells (e.g., Sf9) or mammalian cells (e.g., COS, CHO). In a preferred embodiment according to the present invention, host cell means the cells of E. coli.

Eukaryotic host cells are not limited to use in a particular eukaryotic host cell. A variety of eukaryotic host cells are available, e.g., from depositories such as the American Type Culture Collection (ATCC) and are suitable for use with the vectors of the present invention. The choice of a particular host cell depends to some extent on the particular expression vector used to drive expression of the nucleic adds of the present invention. Eukaryotic host cells include mammalian cells as well as yeast cells.

The imperfect fungus Saccharomyces cerevisiae is the most commonly used eukaryotic microorganism, although a number of other strains are commonly available. For expression in Saccharomyces sp., the plasmid YRp⁷ (ATCC-40053), for example, is commonly used (see e.g., Stinchcomb L. et al. (1979) Nature, 282:39; Kingsman J. al. (1979), Gene, 7:141; S. Tschemper et al. (1980), Gene, 10:157). This plasmid already contains the trp gene, which provides a selectable market for a mutant strain of yeast lacking the ability to grow in tryptophan.

Suitable promoting sequences for use with yeast hosts include the promoters for 3-phosphoglycerate kinase (found on plasmid pAP12BD (ATCC 53231) and described in U.S. Pat. No. 4,935,350, issued Jun. 19, 1990, herein incorporated by reference) or other glycolytic enzymes such as enolase (found on plasmid pAC1 (ATCC 39532)), glyceraldehyde-3-phosphate dehydrogenase (derived from plasmid pHcGAPC1 (ATCC 57090, 57091)), hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase, as well as the alcohol dehydrogenase and pyruvate decarboxylase genes of Zymomonas mobilis (U.S. Pat. No. 5,000,000 issued Mar. 19, 1991, herein incorporated by reference).

Other yeast promoters, which are inducible promoters, having the additional advantage of their transcription being controllable by varying growth conditions, are the promoter regions for alcohol dehydrogenase 2, isocytochrome C, acid phosphatase, degradative enzymes associated with nitrogen metabolism, metallothionein (contained on plasmid vector pCL28XhoLHBPV (ATCC 39475) and described in U.S. Pat. No. 4,840,896, herein incorporated by reference), glyceraldehyde 3-phosphate dehydrogenase, and enzymes responsible for maltose and galactose (e.g. GAL1 found on plasmid pRY121 (ATCC 37658)) utilization. Yeast enhancers such as the UAS Ga1 from Saccharomyces cerevisiae (found in conjunction with the CYC1 promoter on plasmid YEpsec-hI1beta ATCC 67024), also are advantageously used with yeast promoters.

A vector can be introduced into a host cell using any suitable method (e.g., transformation, electroporation, transfection using calcium chloride, rubidium chloride, calcium phosphate, DEAE dextran or other substances, microprojectile bombardment, lipofection, infection or transduction). Transformation relates to the introduction of DNA into an organism so that the DNA is replicable, either as an extrachromosomal element or by chromosomal integration. Methods of transforming bacterial and eukaryotic hosts are well known in the art. Numerous methods, such as nuclear injection, protoplast fusion or by calcium treatment are summarized in Maniatis et al. (2001) supra. Transfection refers to the taking up of a vector by a host cell whether or not any coding sequences are in fact expressed. Successful transfection is generally recognized when any indication or the operation or this vector occurs within the host cell.

Another embodiment of the invention provides a method for the production of the polypeptide of the invention comprising the following steps:
(a) cultivating a host cell of the invention and expressing the nucleic acid under suitable conditions;
(b) isolating the polypeptide with suitable means.

The polypeptides according to the present invention may also be produced by recombinant methods. Recombinant methods are preferred if a high yield is desired. A general method for the construction of any desired DNA sequence is provided, e.g., in Brown J. et al. (1979), Methods in Enzymology, 68:109; Maniatis (1982), supra.

According to the invention an activity-based screening in metagenome library was used as a powerful technique (Lorenz, P et al. (2002), Current Opinion in Biotechnology 13:572-577) to isolated new enzymes from the big diversity of microorganisms found in ramen ecosystem. For this purposes an activity selection technique using alpha-naphthyl acetate as substrate and Fast Blue RR was used. This technology enable screening of 105-109 clones/day from genomes of between 1 and 15,000 microorganisms. In detail, this method is described in the Example 1 below.

The polypeptide can be isolated from the culture medium by conventional procedures including separating the cells from the medium by centrifugation or filtration, if necessary after disruption of the cells, precipitating the proteinaceous components of the supernatant or filtrate by means of a salt, e.g., ammonium sulfate, followed by purification by a variety of chromatographic procedures, e.g., ion exchange chromatography, affinity chromatography or similar art recognized procedures.

Efficient methods for isolating the polypeptide according to the present invention also include to utilize genetic engineering techniques by transforming a suitable host cell with a nucleic acid or a vector provided herein, which encodes the polypeptide and cultivating the resultant recombinant microorganism, preferably E. coli, under conditions suitable for host cell growth and nucleic acid expression, e.g., in the presence of inducer, suitable media supplemented with appropriate salts, growth factors, antibiotic, nutritional supplements, etc.), whereby the nucleic acid is expressed and the encoded polypeptide is produced.

In additional embodiments the polypeptide of the invention can be produced by in vitro translation of a nucleic acid that encodes the polypeptide, by chemical synthesis (e.g., solid phase peptide synthesis) or by any other suitable method.

Skilled artisans will recognize that the polypeptides of the present invention can also be produced by a number of different methods. All of the amino acid sequences of the invention can be made by chemical methods well known in the art, including solid phase peptide synthesis, or recombinant methods. Both methods are described in U.S. Pat. No. 4,617,149, the entirety of which is herein incorporated by reference.

The principles of solid phase chemical synthesis of polypeptides are well known in the art and are described by, e.g., Dugas H. and Penney C. (1981), Bioorganic Chemistry, pages 54-92. For examples, peptides may be synthesized by solid-phase methodology utilizing an Applied Biosystems 430A peptide synthesizer (commercially available from Applied Biosystems, Foster City, Calif.) and synthesis cycles supplied by Applied Biosystems. Protected amino acids, such as t-butoxycarbonyl-protected amino adds, and other reagents are commercially available from many chemical supply houses.

Sequential t-butoxycarbonyl chemistry using double couple protocols are applied to the starting p-methyl benzhydryl amine resins for the production of C-terminal carboxamides. For the production of C-terminal acids, the corresponding pyridine-2-aldoxime methiodide resin is used. Asparagine, glutamine, and arginine are coupled using preformed hydroxy benzotriazole esters. The following side chain protection may be used:

### Arg, Tosyl

Asp, cyclohexyl
Glu, cyclohexyl

### Ser, Benzyl

### Thr, Benzyl

Tyr, 4-bromo carbobenzoxy

Removal of the t-butoxycarbonyl moiety (deprotection) may be accomplished with trifluoroacetic acid (TFA) in methylene chloride. Following completion of the synthesis the peptides may be deprotected and cleaved from the resin with anhydrous hydrogen fluoride containing 10% meta-cresol. Cleavage of the side chain protecting group(s) and of the peptide from the resin is carried out at zero degrees centigrade or below, preferably −20° C. for thirty minutes followed by thirty minutes at 0° C.

After removal of the hydrogen fluoride, the peptide/resin is washed with ether, and the peptide extracted with glacial acetic acid and then lyophilized. Purification is accomplished by size-exclusion chromatography on a Sephadex G-10 (Pharmacia) column in 10% acetic acid.

Another subject of the invention relates to the use of the polypeptide, the nucleic acid, the vector and/or the cell of the invention in consumer products, particularly food products, preferably as a food additive.

When a grain or other plant-derived food or feed component having a substantial non-starch polysaccharide content is used, the energy source availability can be increased by treatment with an esterase according to the invention and a xylanase at a ration of 1 to 200 U/kg for each enzyme, desirably about 10 to about 50 U/kg feed or food. Food or feed can also be supplemented or treated with a combination of an esterase according to the invention and a xylanase to improve nutrition and energy source availability for humans, poultry (e.g., chickens, turkeys, ducks, geese, and other fowl), swine, sheep, cattle, horse, goats, fish (including but not limited to salmon, catfish, tilapia and trout) and shellfish, especially shrimp, and other farmed organisms.

Food or feed ingredients, which can be improved by treatment with a combination of an esterase according to the invention and a xylanase include, for example, wheat, rye, barley, oats, corn, rice, soybean, millet, sorghum, grasses, legumes and other pasture and forage plants. Fresh or dry feed or food components can be treated with a liquid comprising the combination of esterase and xylanase so that the particles of the food or feed are coated with the enzymes. Similarly, wet or dry enzyme compositions can be added to a liquid food or feed composition.

According to the invention of the polypeptide, nucleic acid, vector and/or cell (hereinafter designated as “substances of the invention”) is useful as an animal food additive. For example, wheat presents a potential energy source for, e.g., poultry and swine but it is frequently avoided because of its low energy value relative to corn. The lower energy availability is due to the presence of a significant amount of non-digestible fiber or non-starch polysaccharide ASP). In addition to NSP being unavailable for energy, it also acts as an anti-nutritional factor and reduces digestibility of other components of the diet. The availability of fiber-degrading enzymes that can be added to wheat diets has increased interest in the use of wheat and other grains for poultry and swine rations.

Another subject of the invention relates to the use of the polypeptide, the nucleic acid, the vector and/or the cell of the invention for the treatment in pulp and paper industry.

For example, substances of the invention are useful in the pulp and paper industry for improving the drainability of wood pulp or paper pulp lignin removal from cellulose pulps, for lignin solubilization by cleaving the ester linkages between aromatic acids and lignin and between lignin and hemicelluloses and for disruption of cell wall structure when used in combination with xylanase and other xylan-degrading enzymes in biopulping and biobleaching of pulps.

Thus, an esterase according to the invention, desirably in combination with a cellulase and/or xylanase, for example that from Orpinomyces PC-2, can be used in the pulping and paper recycling industries. The ratio of the esterase to solids is from about 0.1 to about 200 U/kg dry weight, desirably from about 1 to about 100 U/kg, and advantageously from about 10 to about 50 U/kg. This esterase alone or in combination with a xylanase can be formulated as dry material or as liquid concentrate for subsequent use in combination with a source of plant-derived non-starch polysaccharide or poorly digestible plant fiber material to be treated. Such a formulation can be freeze-dried in the case of a dry material or it can be a liquid concentrate. A liquid formulation can contain from about 100 μg to about 50 mg/ml of protein. Reducing agents such as cystine dithiothreitol, dethioerythritol or [beta]-mercaptoethanol can be included to prevent enzyme oxidation and protein stabilizing agents, for example glycerol (0.1% to 10% w/v), sucrose (0.1% to 10% w/v) among others, can be included or an irrelevant protein such as bovine serum albumin or gelatin can also be present. Although the esterases of the present invention are stable, a buffering agent can be added to stabilize the pH.

Another subject of the invention relates to the use of the polypeptide, the nucleic acid, the vector and/or the cell of the invention for the treatment of cellulosic textiles or fabrics, e.g. as an ingredient in detergent compositions or fabric softener compositions. Consequently, the invention relates also to detergent compositions including a polypeptide, nucleic acid, vector and/or cell according to the invention.

The treatment of cellulosic textiles or fabrics includes textile processing or cleaning with a composition comprising a substance of the present invention. Such treating includes, but is not limited to, stonewashing, modifying the texture, feel and/or appearance of cellulose containing fabrics or other techniques used during manufacturing or cleaning/reconditioning of cellulose containing fabrics. Additionally, treating within the context of this invention contemplates the removal of immature cotton from cellulosic fabrics or fibers.

The detergent and solvent resistances or in other words the effect of surfactants on the activity of the polypeptide of the invention was measured. The results are given in FIGS. 2 and 5 and confirm that a polypeptide of the present invention can be employed in a detergent composition. Such a detergent compositions is useful as pre-wash compositions, pre-soak compositions or for cleaning during the regular wash or rinse cycle. Preferably, the detergent compositions of the present invention comprise an effective amount of polypeptide, surfactants, builders, electrolytes, alkalis, antiredeposition agents, bleaching agents, antioxidants, solubilizer and other suitable ingredients known in the art.

An “effective amount” of the polypeptide employed in the detergent compositions of this invention is an amount sufficient to impart the desirable effects and will depend on the extent to which the detergent will be diluted upon addition to water so as to form a wash solution.

“Surfactants” of the detergent composition can be anionic (e.g., linear or branched alkylbenzenesulfonates, alkyl or alkenyl ether sulfates having linear or branched alkyl groups or alkenyl groups, alkyl or alkenyl sulfates, olefinsulfonates and alkanesulfonates), ampholytic (e.g., quaternary ammonium salt sulfonates and betaine-type ampholytic surfactants) or non-ionic surfactants (e.g., polyoxyalkylene ethers, higher fatty acid alkanolamides or alkylene oxide adduct thereof, fatty acid glycerine monoesters). It is also possible to use mixtures of such surfactants.

“Builders” of the detergent composition include, but are not limited to alkali metal salts and alkanolamine salts of the following compounds: phosphates, phosphonates, phosphonocarboxylates, salts of amino acids, aminopolyacetates high molecular electrolytes, non-dissociating polymers, salts of dicarboxylic acids, and aluminosilicate salts.

“Electrolytes” or “alkalis” of the detergent composition include, for example, silicates, carbonates and sulfates as well as organic alkalis such as triethanolamine, diethanolamine, monoethanolamine and triisopropanolamine.

“Antiredeposition agents” of the detergent composition include, for example, polyethylene glycol, polyvinyl alcohol, polyvinylpyrrolidone and carboxymethylcellulose.

“Bleaching agents” of the detergent composition include, for example, potassium monopersulfate, sodium percarbonate, sodium perborate, sodium sulfate/hydrogen peroxide adduct and sodium chloride/hydrogen peroxide adduct or/and a photo-sensitive bleaching dye such as zinc or aluminum salt of sulfonated phthalocyanine further improves the detergenting effects.

“Antioxidants” of the detergent composition include, for example, tert-butyl-hydroxytoluene, 4,4′-butylidenebis(6tert-butyl-3-methylphenol), 2,2′-butylidenebis(6-tert-butyl-4-methylphenol), monostyrenated cresol, distyrenated cresol, monostyrenated phenol, distyrenated phenol and 1,1-bis(4hydroxy-phenyl)cyclohexane.

“Solubilizer” of the detergent composition include, for example, lower alcohols (e.g., ethanol), benzenesulfonate salts, lower alkylbenzenesulfonate salts (e.g., p-toluenesulfonate salts), glycols (e.g., propylene glycol), acetylbenzene-sulfonate salts, acetamides, pyridinedicarboxylic acid amides, benzoate salts and urea.

The detergent compositions of the present invention may be in any suitable form, for example, as a liquid, in granules, in mulsions, in gels, or in pastes. Such forms are well known in the art and are described e.g., in U.S. Pat. No. 5,254,283, which is incorporated herein by reference in its entirety.

The treatment according to the invention also comprises preparing an aqueous solution, which contains an effective amount of the polypeptide, nucleic acid, vector and/or cell of the invention together with other optional ingredients, for example, a surfactant, as described above, a scouting agent and/or a buffer. A buffer can be employed to maintain the pH of the aqueous solution within the desired range. Such suitable buffets are well known in the art. As described above, an effective amount of the polypeptide will depend on the intended purpose of the aqueous solution.

The following figures and examples are thought to illustrate the invention. They should not be constructed to limit the scope of the invention thereon. All references cited by the disclosure of the present application are hereby incorporated in their entirety by reference.

### FIGURES

FIG. 1 shows Table 2 representing substrate specifity of the esterases according to the invention towards several p-nitrophenyl esters and acetylated substrates. The values of esterases encoded by clones pBKR.9, 13, 14, 17, 27, 32, 34, 35, 37, 38, 40, 41, 43, 45, 47, 52 are depicted. The activity is expressed in μmol min⁻¹ g⁻¹ of pure protein.

For an analysis of substrate specifities and acyl chain length preferences of the esterases, the esterolytic activity of esterases towards various p-nitrophenyl esters (p-NP) of fatty acids (C2 to C12) (indicated as p-NPC . . . ) was determined. Results show that esterases preferably hydrolyzed esters of short chain and medium chain (C2-C6) fatty acids. Esters of long-chain fatty acids were poor substrates. pBKR.9 and pBKR.14 showed maximal activity towards fatty acid esters with 6 carbon atoms, pBKR.17 towards butylate p-nitrophenyl ester (=p-NPC₄) and pBKR.13, pBKR.27, pBKR.34, pBKR.38, pBKR.40, pBKR.41, pBKR.43, pBKR45, pBKR.47 and pBKR.52 were highly specific for p-Npacetate (=p-NPC₂). pBKR.35 was the less active enzymes towards p-nitrophenyl esters. The lack of esterase activity against long-chain p-nitrophenyl fatty acid esters exclude the possibility than the enzymes may have been lipases.

Furthermore, the esterases were tested for their ability to hydrolyze various carbohydrate acetyl-esters, i.e., glucose, xylose or cellulose acetate esters. As shown, some of the esterases showed acetyl xylan esterase activity, i.e. pBKR.13, pBKR.17, pBKR.35, pBKR.37, pBKR.38, pBKR.41, pBKR.43 and pBKR.47. A comparative analysis shows that pBKR.35 was more active towards glucose, xylose or cellulose acetate esters, than against p-nitrophenyl esters. The substrate specificity results are consistent with the classification of pBKR.13, pBKR.17, pBKR.35, pBKR.37, pBKR.38, pBKR.41, pBKR.43 and pBKR.47 as esterases associated with the degradation of complex polysaccharides.

FIG. 2 shows Table 3 representing an overview over the biochemical properties of the esterases of the invention. The values for esterases of clones pBKR.9, 13, 14, 17, 27, 32, 34, 35, 38, 40, 41, 43, 45, 47 and 52 are given. Reactions were performed at the optimum pH and temperature for each clone using different substrates: p-NPA, p-NPB or glucose pentaacetate. p-NPA was used as substrate for clones pBKR.13, pBKR.27, pBKR.34, pBKR.38, pBKR.40, pBKR.41, pBKR.43, pBKR.45, pBKR.47 and pBKR.52, p-NPB was used as substrate for pBKR.9, pBKR.14 and pBKR.17 and glucose pentaacetate was used as substrate for pBKR.32, pBKR.35 and pBKR.37. For each clone the following data are given:

maximum pH at which the remaining activity after 24 h incubation at the indicate pH is >50% (percentage activity at the indicate pH is shown in parenthesis) (column “Stable at pH”). The esterases were highly active at pHs between 7.5 to 10.0. pBKR.13, pBKR.14, pBKR.34, pBKR.38, pBKR.40, pBKR43 and pBKR.45 show maximum activity at pH 7.5-8.5 with 20% of the maximum activity at pH>10 and pBKR.9, pBKR.17, pBKR.27, pBKR.32, pBKR.35, pBKR.41, pBK47 and pBKR.52 shows maximum activity at pH 9.5-10.0 with activities at pH between 10.0-11.0>83%.
maximum temperature at which the remaining activity after 2 h incubation is >90% (column “Stable at Temperature”). The average temperature for all clones was in the range 40-60° C. However, the esterases are also highly active at a temperature as low as 4° C. (75% of the activity showed at the optimal temperature). At 60° C., the activity in the range 27.1% to 38.9% of the activity showed at the optimal temperature.
specific activity at the optimum pH and the optimum temperature using p-NPB as esterase substrate (for details see Examples). The specific activity is expressed in μmol min⁻¹ g⁻¹. pBKR.34, 17 and 40 showed the highest specific activities, whereas pBKR.9 and 35, showing a maximum activity at high pHs (pH 12.0 and 11.0), showed low specific activities.
cation dependence: all indicated cations (NH4⁺, Na⁺, K⁺, Ca²⁺, Mg²⁺, Li⁺, Zn²⁺, Sr²⁺ and Co²⁺) were added as chloride salts and tested at a concentration of 10 to 100 mM Nearly all clones exhibited no salt dependence, i.e. the purified esterases of the invention showed esterolytic activity without addition of any metal ions. Moreover, in the majority of the esterases the addition of EDTA did not result in a decrease in esterase activity, indicating that esterases were independent of divalent cations. Thus, clones pBKR.9, 13, 27, 32, 35, 38, 41, 43 and 45 showed no cation dependence at all, whereas pBKR 34 (showing the highest specific activity) was slightly activated (1.4 fold) by NH4⁺ at >25 mM and pBKR 52 was slightly activated (1.1-1.6 fold) by mono- and divalent cations. In contrast, pBKR 17 and pBKR 40 (showing the second and third highest specific activity) were inhibited by Sr²⁺ or Co²⁺ at concentration>75 mM, respectively. pBKR 40 was strongly inhibited by Mg²⁺, Zn²⁺ at concentration>25 mM.
detergent resistance: the effect of various detergents, i.e. Triton X-100 and SDS (sodium dodecylsulphate) at concentration of 1% w/v or 50 mM, respectively, Tween20 and Tween80 (from 0.05 to 3% v/v), on the esterolytic activity of the esterases according to the invention, was analyzed. Unlike otherwise indicated the detergent resistance is given by the remaining esterase activity in the corresponding buffer (optimum pH and T) containing the detergent. Activity was measured after 2 h incubation and compared with a control reaction in buffer lacking detergent. The level of activity of the enzyme after 24 h incubation with each detergent did not display significant differences from the activity observed when the activity was measured immediately. All esterases of the invention showed a high detergent resistance except pBKR.35. pBKR.14 was not influenced by Triton X-100 between 1-3% whereas pBKR.34, 40, 45, 47 and 52 retains more than 85% activity up to 3% Triton X-100. pBKR.9, 13, 17, 27, 32, 41, 43 were only slightly inhibited (from 17% up to 38%) by used detergents. The most resistant to SDS up to 50 mM, were pBKR.34 and pBKR.40

FIG. 3 shows the determination of pH optima of esterase according to the invention. Reactions were carried out for 2 min at 40° C. in the following 100 mM buffer solutions: citrate (circles), HEPES (squares) and Tris-HCl (triangles). p-NPB was used as substrate. The relative activity is expressed in %. The results confirmed the data of Table 3 (FIG. 2).

FIG. 4 shows Table 4 representing a further overview of the effects of cations and detergents on the activity of esterases of the invention, which show an effect on cations up to a concentration of 75 mM. The values for clones pBKR. 14, 17, 34, 40, 47 and 52 are given. The concentrations of the additives are given in mM and the effects on esterase activity are indicated as percentage.

FIG. 5 shows Table 5 representing the effect of solvents on esterase activity. Since it is well known that organic solvents affect the enzyme activities of different lipases and esterases, which are different from each others, the effects on the activity of esterases according to the invention was analyzed. Data of clones pBKR.9, 13, 14, 17, 27, 34, 35, 40, 41, 43, 45 and 47 are given. The effects on esterase activity are indicated as percentage. The purified esterases were incubated with various water-miscible and immiscible solvents at 4° C. from 2 to 120 min. In general, when activity and stability towards organic solvents is tested, the activity is to be measured in a broad range of time, to check whether the enzyme is stable or not. If the enzyme is not stable the activity after 2 min (the minimum time for the esterase assay) will be higher than after longer incubation. This time incubation depends on the analysis (1, 2, 4, 24 h). According to the invention the time incubation is limited to 120 min, because 2 hours is more than the time chemical reactions like that are applied in industry or chemical synthesis. In the case of rumen esterase the activity did not differ between 2 and 180 min, and this indicates that rumen esterases are stable. Of course, according to the invention, it is also possible to analyse the activity after longer times.

A concentration range from 30 to 90% (v/v) was used. The results showed that the majority of esterase were active and stable in polar solvent, which were normally used in biocatalysis. Slight activation of esterolytic activity was detected in the presence of medium and high concentrations of acetonitrile (up to 2.4 fold), tert-amylalcohol (up to 2.4 fold) and dimethylsulfoxide (up to 1.6 fold). The most stable esterases were those encoded by clones pBKR.9, pBKR.13, pBKR.35, whereas the most susceptible clone was pBKR.14. Furthermore, all the enzymes were also active and stable in non-polar solvents, such as hexane, iso-octane, toluene and medium polar solvents, i.e., tert-butyl alcohol, (data not shown).

FIGS. 6 to 8 show Table 6 to 8 representing the substrate selectivity of the esterases of the invention. A rapid screen method was used reported by Janes et al. (1998), Chem. Eur. J. 4: 2317-2324. 6, to map substrate selectivity. A chiral ester library of 13 pairs of enantiomers was screened in 96-well plates using EPPS buffer, pH 8.0 (at pH 8.0 majority of esterases of the invention showed maximum activity), and phenol red as pH indicator. The activity as well as the true selectivity were determined in the presence of resorufin acetate as reference compound (Janes et al. (1998), supra, Man Fai Lui et al. (2001), supra). The chiral ester library was chosen to identify the acyl chain length preferred by the esterases of the invention as well as the ability of the esterases to hydrolyze hindered or charged group, including lactones, primary and secondary alcohols and aromatic and non-aromatic compounds containing carboxylic acids with a stereocenter alpha or beta to carbonyl. The results show high esterolytic activities towards lactones and chiral carboxylic acid with a stereocenter alpha to carbonyl (from 281 to 7111 μmol min⁻¹ g⁻¹) (see Table 7), followed by primary or secondary alcohols (from 299 to 5963 μmol min⁻¹ g⁻¹) (see Table 6) and in less extension towards chiral esters with stereocenter ? to carbonyl (from 303 to 3946 μmol min⁻¹ g⁻¹) (see Table 8).

FIG. 6 shows Table 6 representing the esterolytic activities of the esterases towards primary or secondary alcohols. For the resolution of primary and secondary alcohols (neomenthyl acetate and menthyl acetate) were found that esterases encoded by pBKR.13, pBKR.14, pBKR.38, pBKR.40 and pBKR.45 showed high enantioselectivity (E from 20 to more than 100) towards both aromatic and non-aromatic esters, whereas pBKR.17 and pBKR.43 were more specific for non-aromatic alcohols. In contrast, pBKR.41 hydrolyzed only aromatic chiral esters. To note from Table 1 (FIG. 9), is the resolution of solketal esters where the enantiomeric ratio showed by several clones were quite high, i.e. pBKR.17, pBKR.34 and pBKR.40 (E values of 8.4, 18.5 and 12.6, respectively), which are higher or similar than the best reported values in the literature, i.e. 14.8 for horse liver esterase (Altschul et al. (1997) Gapped BLAST and PSI-BLAST: a new generation of protein database search programs, Nucleic Acids Res. 25: 3389-3402; Lorenz, et al. (2002), Current Opinion in Biotechnology 13: 572-577) and 9.0 for Bulkhoderia cepacia lipase (Weissfloch et al., (1995), J. Org. Chem. 60: 6959-6969). Solketal esters are important intermediate compounds for AIDS drugs development.

FIG. 7 shows Table 7 representing the esterolytic activities of the esterases towards lactones and chiral carboxylic acid with a stereocenter alpha to carbonyl. Six esterases, pBKR.17 (E>100), pBKR.34 E>100), pBKR.40 (E˜84), pBKR.41 (E˜19) and pBKR.45 E˜14) showed high enantiomeric ratio towards esters of chiral carboxylic acids (stereocenter alpha to carbonyl). Positive esterases for the resolution of lactones (pantolactone and dihydro-5-hydroxymethyl-2(3H)-furanone) were pBKR.41 (E˜15 for pantolactone and 64 for dihydro-5-hydroxymethyl-2(3H)-furanone) and pBKR.43 (E˜8.3 for pantolactone and 28.0 for dihydro-5-hydroxymethyl-2(3H)-furanone.

FIG. 8 shows Table 8 representing the esterolytic activities of the esterases towards chiral esters with stereocenter beta to carbonyl. Six esterases, in detail: pBKR.13, pBKR.27, pBKR.38, pBKR.41 and pBKR.43, showed E-values>20 for the resolution of chiral carboxylic acid with a stereocenter beta to carbonyl. From these clones, pBKR.13, pBKR.18, pBKR.27 and pBKR.38 were useful for resolution of aromatic compounds containing chiral carboxylic acid with a stereocenter beta to carbonyl, involving amino acid derivatives, whereas pBKR.41 and pBKR.43 were highly useful for industrial resolutions of chiral carboxylic acid with a stereocenter beta to carbonyl, involving lactic derivatives.

FIG. 9 shows Table 1 representing a comparison of specific activity of rumen esterases of the invention with commercial esterases.

FIGS. 10 to 33 shows the coding nucleic acid sequences (FIGS. 10 to 21) and the deduced amino acid sequences (FIGS. 22 to 33) of the clones of the invention. The determination of the translation start codon was based on the fact that this was the longest ORF observed as well as there was a typical signal sequence of the predicted protein. N-terminus sequenced matched the deduced amino acid sequence. Parallel to this, the determination of the translation start codon was done using the Hidden Markov Model (HMM), based web tool (http://opal.biology.gatech.edu/GeneMark/heuristic_hmm2.cgi) and then confirmed by the N-terminal peptide sequencing. Predicted peptide sequences of the enzymes (esterases) confirmed a low identity (from 30 to less than 10%) and a low similarity (from 60% to 40% and less) of deduced amino acid sequences of the cloned DNA fragments to sequences of known ester hydrolases classified in different families after comparison with the sequences, which were available in the National Center for Biotechnology Information (NCBI) database.

The data suggest that pBKR.13, pBKR.27, pBKR.40, pBKR.41, pBKR.43, belong to a new family of ester hydrolases. Some of the esterases showed similarity to acetylxylane esterases pBKR.32, pBKR.45). All the esterases except those from pBKR.44 and pBKR.45 contained either the sequence, Gly-X-Ser-X-Gly (with X an arbitrary amino acid residue), or Gly-Ser-Asp-(Lys) found in most serine hydrolases of this superfamily (Ollis et al., 1992; Jaeger et al., 1994; 1999). The highest degree of homology with other ester hydrolases was found around the above motifs.

FIGS. 10 to 21 show the nucleic acid sequences of the positive clones obtained from the genomic library constructed from ruminal ecosystem. In detail

FIG. 10 shows the nucleic acid sequence of clone pBKR 09,

FIG. 11 shows the identical nucleic acid sequence of clones pBKR 13 and pBKR 52,

FIG. 12 shows the nucleic acid sequence of clone pBKR 14,

FIG. 13 shows the nucleic acid sequence of clone pBKR 17,

FIG. 14 shows the nucleic acid sequence of clone pBKR 27,

FIG. 15 shows the nucleic acid sequence of clone pBKR 34,

FIG. 16 shows the nucleic acid sequence of clone pBKR 35,

FIG. 17 shows the nucleic acid sequence of clone pBKR 40,

FIG. 18 shows the nucleic acid sequence of clone pBKR 41,

FIG. 19 shows the nucleic acid sequence of clone pBKR 43,

FIG. 20 shows the nucleic acid sequence of clone pBKR 44,

FIG. 21 shows the identical nucleic acid sequence of clones pBKR 45 and pBKR 48,

FIGS. 22 to 33 show the amino acid sequences of the positive clones obtained from the genomic library constructed from ruminal ecosystem. In detail

FIG. 22 shows the amino add sequence of clone pBKR 09,

FIG. 23 shows the identical amino acid sequence of clones pBKR 13 and pBKR 52,

FIG. 24 shows the amino add sequence of clone pBKR 14,

FIG. 25 shows the amino acid sequence of clone pBKR 17 (polypeptide and mature protein),

FIG. 26 shows the amino acid sequence of clone pBKR 27,

FIG. 27 shows the amino acid sequence of clone pBKR 34 (polypeptide and mature protein),

FIG. 28 shows the amino acid sequence of clone pBKR 35,

FIG. 29 shows the amino acid sequence of clone pBKR 40,

FIG. 30 shows the amino acid sequence of clone pBKR 41,

FIG. 31 shows the amino acid sequence of clone pBKR 43,

FIG. 32 shows the amino acid sequence of clone pBKR 44,

FIG. 33 shows the identical amino acid sequence of clones pBKR 45 and pBKR 48,

### EXAMPLES

### Materials and Buffers

p-nitrophenyl esters, triacetin, tributyrin, Fast Blue RR, α-naphthyl acetate and butyrate, indoxyl acetate, olive oil emulsion, resorufin esters and phenyl ethanol were purchased from Sigma Chemical Co. (St. Louis, Mo., USA). Molecular mass markets for SDS- and native-PAGE were provided from Novagen (EMD Biosciences, Inc. La Jolla, Calif., USA) and Amersham Pharmacia Biotech (Little Chalfont, United Kingdom), respectively. Unless otherwise noted, esters for the substrate library were purchased from Aldrich (Oakville, ON) or Fluka (Oakville, ON). Restriction and modifying enzymes were purchased from New England Biolabs. DNase I grade II, was from Boehringer Mannheim, DE. Chromatographic media and LMW calibration kit for native electrophoresis, were from Amersham Pharmacia Biotech. The following buffers were used: buffer A, 50 mM Tris-HCl buffer, pH 7.0; buffer B, 50 mM Tris-HCl, pH 7.0, 1 M NaCl; buffer C, 50 mM Tris-HCl buffer, pH 7.0, 1 M (NH₄)₂SO₄; Buffer D, 10 mM Tris-HCl buffer, pH 7.0, Buffer E, 10 mM Tris-HCl, pH 7.0 150 mM NaCl. All operations were performed at 4° C. to maintain the stability of all proteins during purification.

### Example 1

### Construction of Environmental DNA Libraries and Screening for Genes Conferring Esterase Activity

An environmental DNA library was constructed from rumen content of New Zealand dairy cows, using Escherichia coli XL1-Blue MRF′ strain as a host. The DNA was isolated from the samples using the phenol method and the genomic library in bacteriophage lambda (4×10⁸ phage particles, average insert size 7.5 kb) was created using ZAP Express Kit (Stratagene) according to producers' protocols. Esterase-positive clones were selected as follows. After infection of E. coli and consequent incubation, the plates (22.5×22.5 cm) containing about 7 000 phage clones were overlaid with 20 ml of a water solution containing 320 μl of α-naphthyl acetate (20 mg/ml in dimethylsulfoxide), 5 mM IPTG and 320 μl of Fast Blue RR (80 mg/ml in dimethylsulfoxide). Positive clones exhibited a brown halo after about 30-120 seg of incubation. Those were picked and the separate positive clones were isolated after consequent phage particles dilution, E. coli infection and halo detection. From the selected phages, the pBIC-CMV plasmids have been excised using co-infection with helper phage, f1 (according to Stratagene protocols), and the insert DNA was sequenced from both ends by using universal primers. Internal primers were made from the original sequence and used to sequence both strands of the insert completely. The plasmids were isolated and analyzed by restriction enzyme analysis.

### Example 2

### Expression and Purification of Esterases in E. coli

For the expression of rumen esterases, the corresponding plasmids that bears the esterase genes in the orientation that enables their expression from Plac-promoter of the plasmids were chosen. E. coli XL1-Blue MRF′ cells bearing pBKR.9, pBKR.13, pBKR.14, pBKR.17, pBKR.27, pBKR.34, pBKR.35, pBKR.40, pBKR.41, pBKR.43, pBKR.45, pBKR.47 were grown in LB medium with 50 μg/ml kanamycin at 37° C. The development of the optical density at 600 nm was followed in time. Once the cultures had reached a OD600 nm of 1.5, the production of the recombinant protein was induced by addition of isopropyl-beta-D-galactopyranoside to a final concentration of 2 mM. 3 h after induction, bacterial cells were harvested and resuspended in buffer A, which contained 1 protease inhibitor cocktail tablet (Roche) and DNase I grade II, incubated on ice for 30-45 min, and then sonicated for 4 min total time. The soluble fraction was separated from insoluble debris by centrifugation (10,000×g, 30 min, 4° C.), dialyzed overnight against buffer A, concentrated by ultrafiltration on a Centricon YM-10 membrane (Amicon, Millipore) to a total volume of 1000 μl and purified by preparative non-denaturing PAGE (5-15% polyacrylamide), at 45 V constant power at 4° C., according to the manufacturer's (Bio-Rad) protocol. The gel region containing the active esterases, detected in a parallel track by activity staining (using Fast blue RR and α-naphthyl acetate: see above), was excised, suspended in two volumes of buffer A and homogenized in a glass tissue homogenizer. The eluate, obtained after removal of polyacrylamide by centrifugation at 4500 g at 4° C. for 15 min, was concentrated by ultrafiltration on a Centricon YM-10 (Amicon, Millipore), to a total volume of 1000 μl. Sample was further purified on a Superose 12 HR 10/30 gel filtration column pre-equilibrated with buffer A containing 150 mM NaCl. Separation was performed at 4° C. at a flow rate of 0.5 ml/min. The following standards were used to calibrate the column: Ribonuclease A (13.7 kDa), Chymotrypsinogen A (25 kDa), Ovalbumin (43 kDa), Bovine serum albumin (67 kDa), Gamma globulin (158 kDa) and Ferritin (440 kDa). All operations were performed at 4° C. to maintain the stability of all the proteins during purification. The purified recombinant esterases were dialysed versus buffer A and stored at −20° C., at a concentration of 50 μM, until use. The N-terminal and several internal fragments sequencing was performed to corroborate the identity of the proteins.

### Example 3

### Esterase Assay

Esterase activity using p-nitrophenyl esters ranging from acetate to laurate as substrates was assayed spectrophotometrically. Briefly, the enzyme activity using p-nitrophenyl esters ranging from acetate to laurate as substrates was assayed by the addition of 5 μl esterase containing solution (50 μM) to 150 μl of 16 mM p-nitrophenyl ester (Sigma) stock solution (in isopropanol), in 2850 μl of a mixture containing 0.1 M of the corresponding buffer, 15% acetonitrile, and 0.038 mM Triton X-100. The esterase reaction was monitored spectrophotometrically at 405 nm. One unit of enzymatic activity was defined as the amount of protein releasing 1 μmol of p-nitrophenoxide/min from p-nitrophenyl ester at the indicated temperature and pH. The release of acetate from acetylated substrates (glucose pentaacetate, tri-O-acetyl-D-galactal, xylose tetraacetate and ABX-acetylated birchwood xylan), was measured using a Boehringer Mannheim acetic acid assay kit (no. 148261), in a mixture containing 0.1 M of the corresponding buffer and 83 μM final concentration of pure esterase. Ferulic ester hydrolase activity towards FAXX, O-[5-O-(trans-feruloyl)-α-L-arabinofuranosyl-(1,3)-O-β-D-xylopyranosyl-(1,4)-D-xylo-pyranose], was assay in a mixture containing 0.1 M of the corresponding buffer and 83 μM final concentration of pure esterase, and enzymatic products release were analysed by HPLC using a reverse phase analytical column (8×100 mm, Waters Nova-Pak C18 Radial-PAK, 4 μm pore size) with isocratic elution by water:acetic acid:butanol (350:1:7) at 2 ml min⁻¹ and detection at 254 nm. One unit of enzyme is defined as the amount of enzyme liberating 1 μmol product min⁻¹, under experimental conditions. All values were determined in triplicate and were corrected considering the autohydrolysis of the substrate. Hydrolytic activity was also determined by titrating free fatty acids released by hydrolysis of triacetin, tripropionin, tributyrin and olive oil, using the pH-stat method, as described previously (San Clemente and Valdegra, 1967). The hydrolysis of substrates was assayed titrimetrically at the optimum pH and temperature in a pH-stat (Mettler, model DL50) using 0.01 M NaOH as titrant. The reaction mixture (20 ml) contained the substrate, 0.15 M NaCl and 0.09% (v/v) acetonitrile. One lipase unit is defined as the amount of enzyme liberating 1 μmol of free fatty acid per minute. Unlike otherwise indicated the standard esterase assay used in this study was: 0.8 mM p-NPA, 100 mM Tris-HCl, pH 8.0, 40° C.

### Example 4

### Temperature and pH Effects on Esterase Activity

Optimal pH and temperature were determined in the range pH 5.5-12.0 (100 mM sodium citrate, pH 5.5; MES, pH 5.5-7.0; HEPES, pH 7.0-8.0; Tris-HCl, pH 8.0-9.0 and glycine-NaOH, pH 9.0-12) and 4-80° C., respectively. In both cases determination was made using p-nitrophenyl acetate in 2 min assays. For optimal temperature determination, 100 mM Tris-HCl buffer, pH 8.0, was used. For determination of temperature and pH stability, 100 μl aliquots were withdrawn at times and remaining esterase activity was measured using the standard assay. Residual activity was monitored by taken the activity at the indicated temperature and pH as 100%. To study the effect of cations, inhibitors, solvents and surfactants in esterase activity, 100 mM Tris-HCl buffer, pH 8.0 supplemented with the corresponding chemical, was used. Activity measurements were carried out immediately and after 30 min of incubation at 40° C. The esterase activity was assayed using the standard esterase assay. In the cases of inhibitors, the enzyme was incubated for 5 min with different concentrations of the inhibitor (o-10 mM). The reaction was stopped by chilling on ice, and aliquots were assayed by the standard assay. The esterase reaction was monitored in at 405 nm, and the residual activity determined quantitatively with respect to a control, as described above. All values were determined in triplicate and were corrected considering the autohydrolysis of the substrate. For the study of organic solvents on activity and stability of the esterase, the enzyme was incubated at 40° C. in 100 mM Tris-HCl, pH 8.0 buffer containing the indicated organic solvent in a concentration ranging from 0 to 90% v/v. Activity measurements were carried out immediately and after 12 h of incubation at the given temperature. Residual activity was determined with 0.8 mM p-nitrophenyl acetate or butyrate using the standard esterase assay and expressed as percent of the control value (without addition of organic solvent).

### Example 5

### Effect of Various Chemicals on Esterase Activity

To study the effect of cations, inhibitors, solvents and surfactants in esterase activity, the conditions were as follows. Salts were used at concentration ranging from 1 to 125 mM. The effects of detergents on the esterase activity was analysed by adding of 1% (wt/vol) detergent to the enzyme solution. Activity measurements were carried out immediately and after 30 min of incubation at 25° C. The enzyme activity was assayed as described above in Tris-HCl at the optimum pH. The esterase reaction was monitored at 405 nm and the residual activity was determined quantitatively with respect to a control, as described above. All values were determined in triplicate and were corrected considering the autohydrolysis of the substrate. In the cases of inhibitors the enzyme was incubated at the optimum temperature and pH for 5 min with different concentrations of the inhibitor. The reaction was stopped by chilling on ice and aliquots were assayed by the standard assay. For the study of organic solvents on activity and stability of the esterase the enzyme was incubated at the optimum temperature in standard buffer containing the indicate organic solvent in a concentration ranging from 0 to 90%. Activity measurements were carried out immediately and after 12 h of incubation at the given temperature. Residual activity was determined with 0.4 mM p-nitrophenyl acetate or butyrate in Tris-HCl buffer at the optimum pH and temperature and expressed as percent of the control value (without addition of organic solvent).

### Example 6

### Positional Specificity and Enantioselectivity of Hydrolases

The hydrolysis of enantiomerically pure esters was measured colorimetrically in 5.0 mM EPPS buffer (N-(2-hydroxyethyl)piperazine-N′-(3-propanesulfonic acid), pH 8.0 and phenol red, on a 96-well microtiter plate and by using 1 to 5 μg of pure protein, in each assay (Man Lai Liu et al., 2001; Janes et al., 1998).

### Example 7

### Assays and Other Methods

The protein concentration was determined by the Bradford dye-binding method with a BioRad Protein Assay Kit with bovine serum albumin as standard Bradford, M M (1976), Anal Biochem 72: 248-254). SDS-PAGE and native electrophoresis were performed according to Laemmli, U K (1970), Nature 227: 680-685. For NH₂-terminal amino acid sequencing, the purified proteins were subjected to PAGE in the presence of sodium dodecyl sulfate (SDS), and protein bands were blotted to a polyvinylidene difluoride membrane (Millipore Corp.) using semidry blot transfer apparatus according to the manufacturer's instructions. The blotted membrane was stained with Coomassie Brilliant Blue R250 and after destaining with 40% methanol/10% acetic acid the bands were cut out and processed for N-terminal amino acid sequence.

### Example 7a

A chemically stable esterase from rumen metagenome (R.34) was characterized by a number of experiments.

### Protein Purification and Biochemical Characterization of Wild Type R.34 Esterase

1. Sequence Analysis and Expression of R.34 Esterase in Escherichia coli

A novel esterase, R.34, was retrieved from the bacteriophage lambda-based expression library created from DNA isolated from rumen fluid of one New Zealand dairy cow, after screening on indicator plates that contained αNA. Sequence analysis revealed an ORF of 5700 bp encoding a polypeptide of 273 residues. Analysis of the deduced amino-acid sequence is consistent with a protein of Mᵣ 25.810 Da and an isoelectric point of 4.57. The amino-acid sequence shares 49% identity (top hit), to the sequence of putative xylanase from Bacteroides thetaiotaomicron VPI-548. It also shared with beta-1,4-D-xylanase from Butyrivibrio fibrisolvens (22% identity), acetyl esterase family enzyme from Clostridium acetobutylicum ATCC 824 (25%), and esterases/lipases from Bifidobacterium longum D5010A (39%), Lactobacillus plantarum WCFS1 (33%) and Magnetospirdium magnetotacticum MS-1 (35%). Lipases typically have a Ser-Asp-His catalytic triad where the active site serine is located within the middle of the conserved consensus GXSXG or GDS(L) motifs (J. L. Arpigny, K. E. Jaeger. Biochem. J. 1999, 343, 177-183). R.34 contained in their sequences the motif GDS(L) (FIG. 44), which is typical for family II of ester hydrolases (J. L. Arpigny, K. E. Jaeger. Biochem. J. 1999, 343, 177-183). Sequence inspection allowed the identification of residues Ser₁₃₇, Asp₂₁₅ and His₂₄₇ as the catalytic residues of R.34 esterase (FIG. 44).

### 2. Characterization of Recombinant R.34 Esterase

In order to study in more detail the biochemical properties and substrate specificity of the esterase, R.34 was expressed as a carboxyl-terminal 6×His tag from pCRR.34 in E. coli TOP10 and affinity purified using a Ni-Sepharose column. About 2.2 trig of pure recombinant protein per g wet weight cells were recovered by a one-step purification method involving metal-chelating chromatography (FIG. 47). Summary of the properties, subunit composition and putative catalytic triad of the recombinant R.34 is shown in Table S1.

Subunit composition: The subunit structure of the purified esterase was deduced from the ration of the experimentally determined molecular weight of the undenatured protein (assessed by polyacrylamide gel electrophoresis and gel filtration) and its subunit molecular mass determined by translation of it gene sequence, to be monomeric (25810:26000 Da).

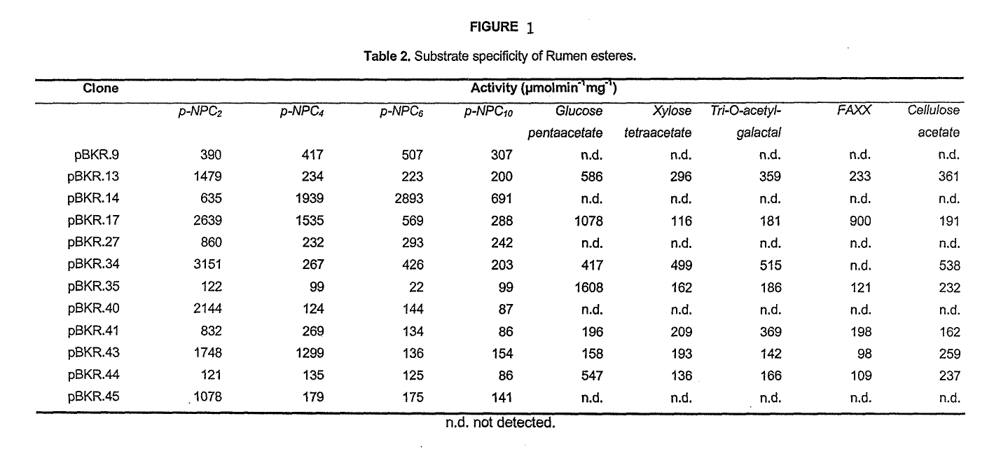

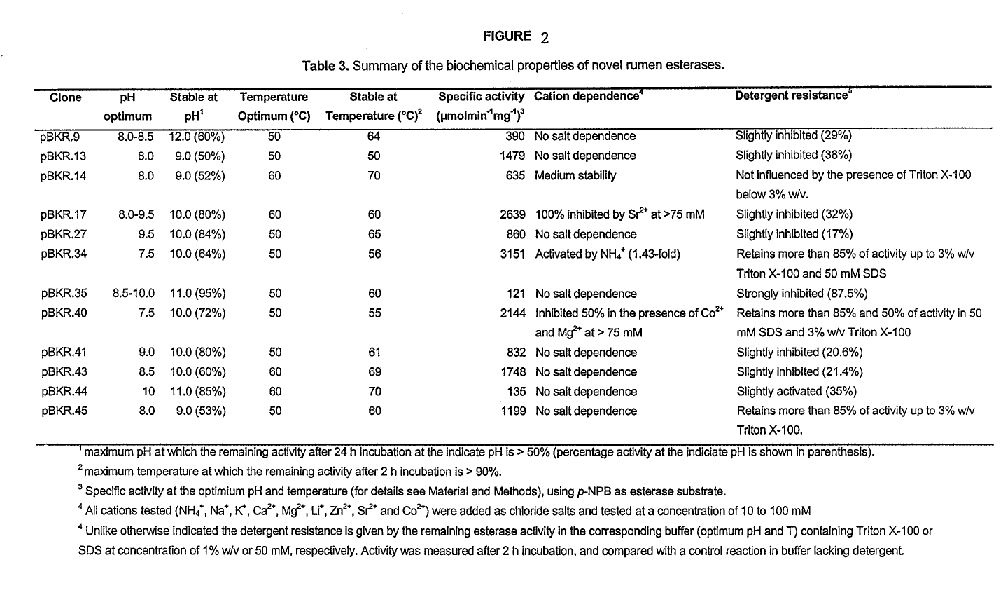

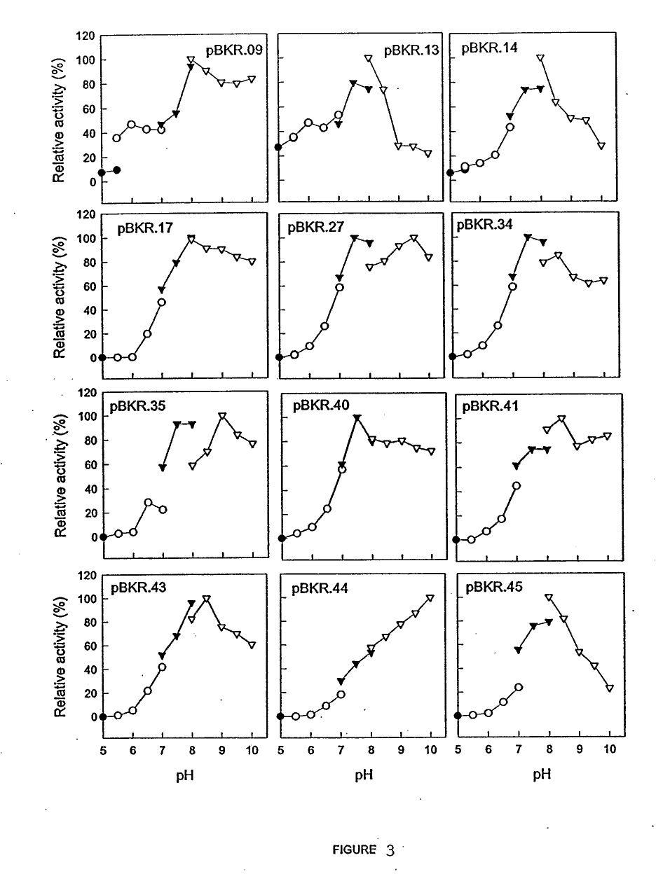

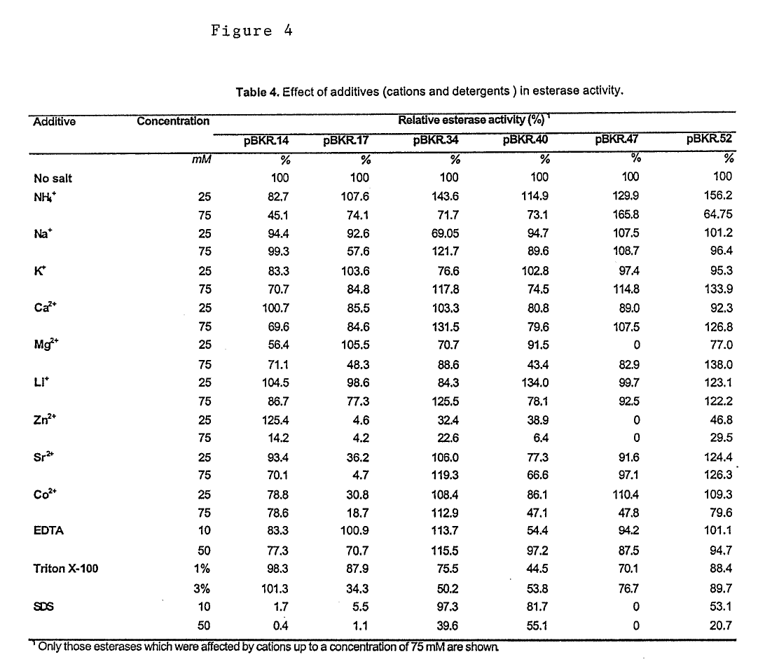

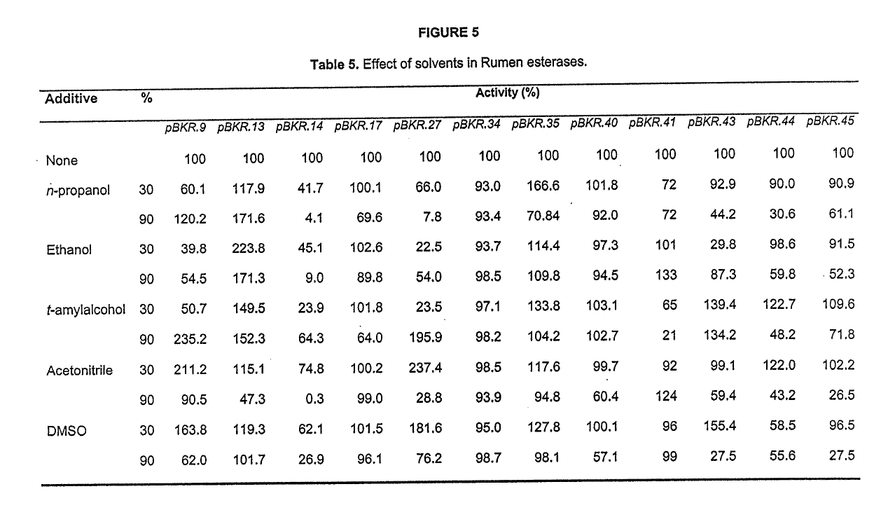

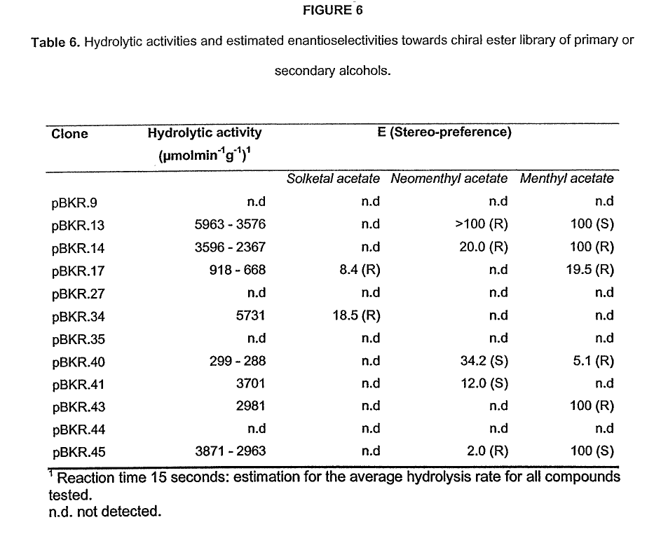

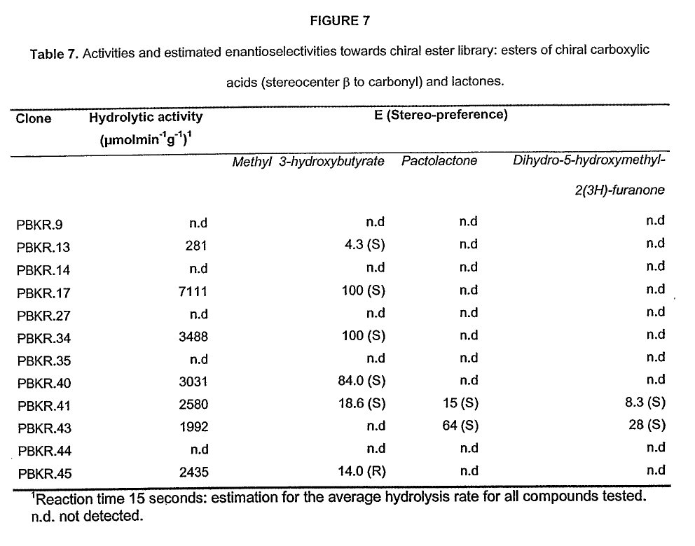

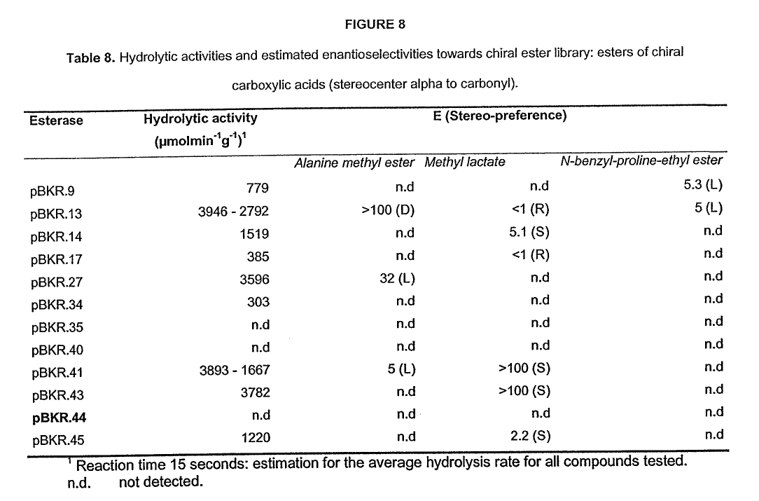

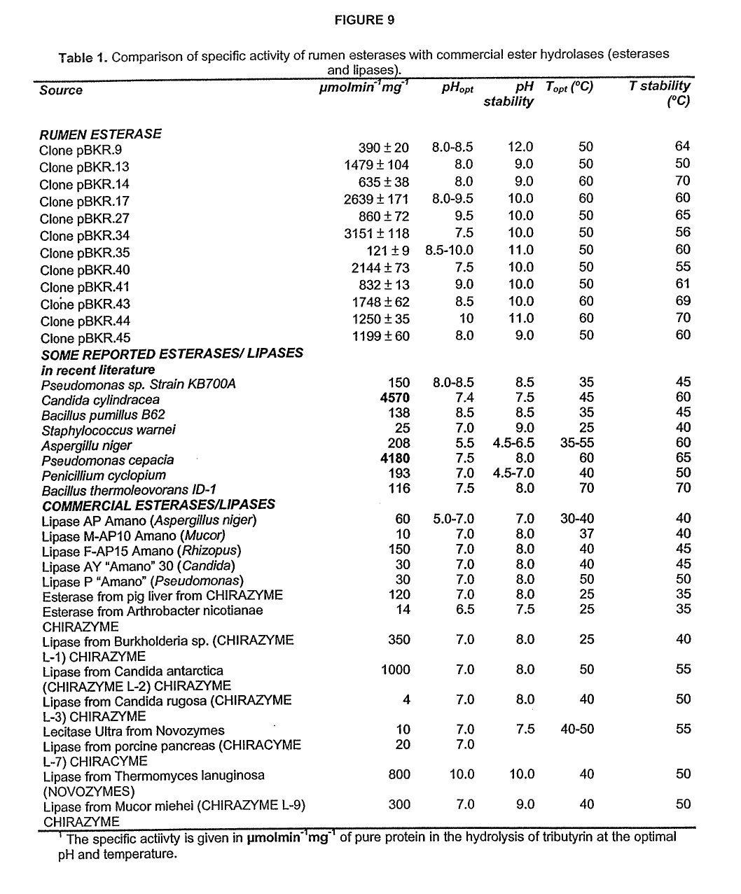

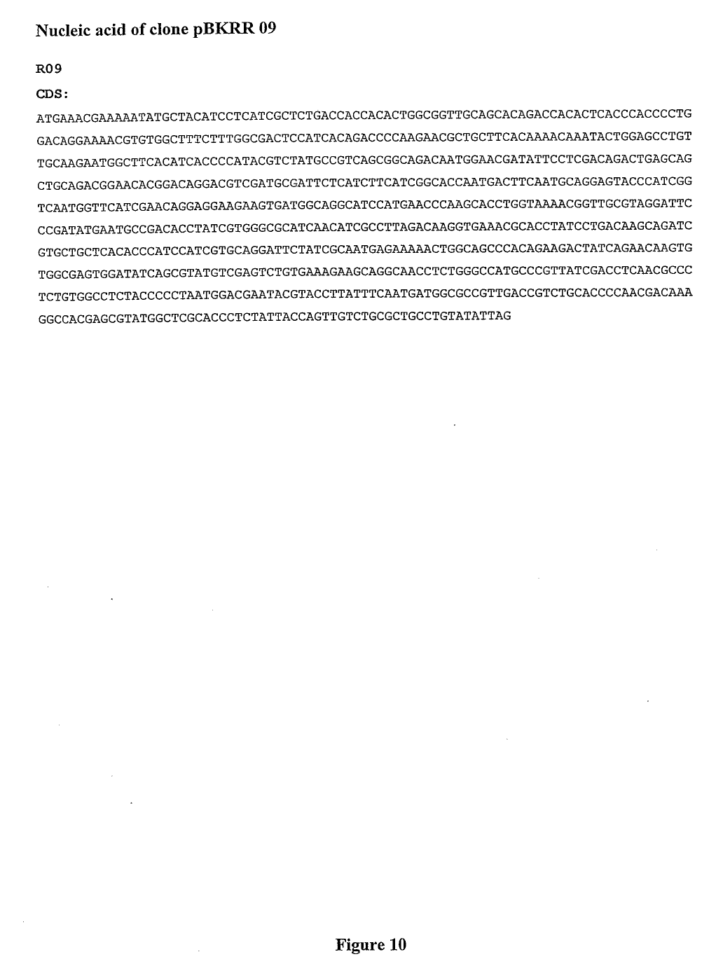

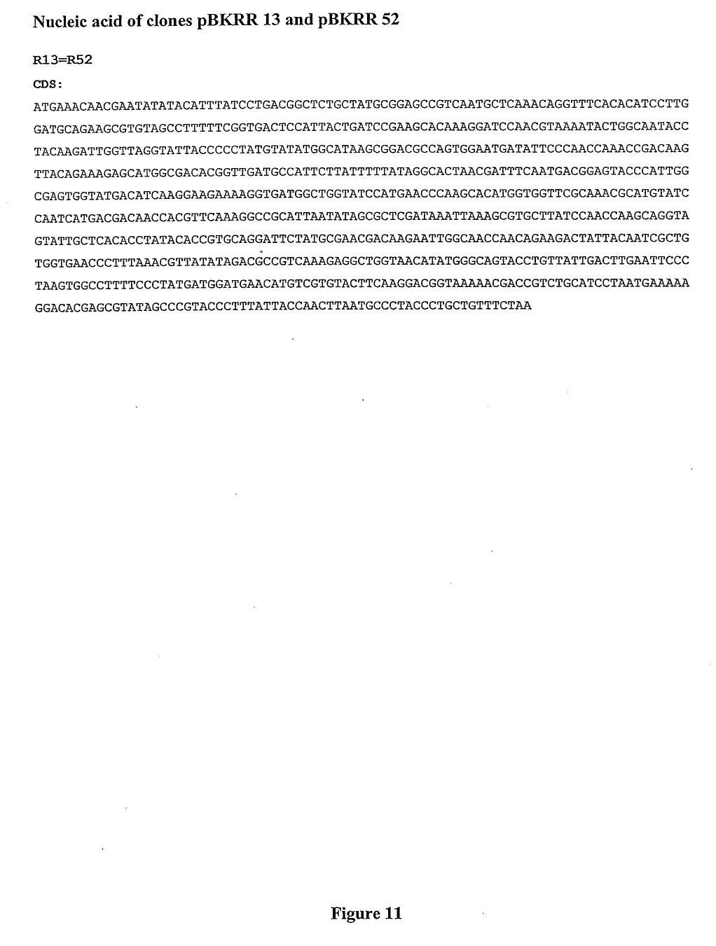

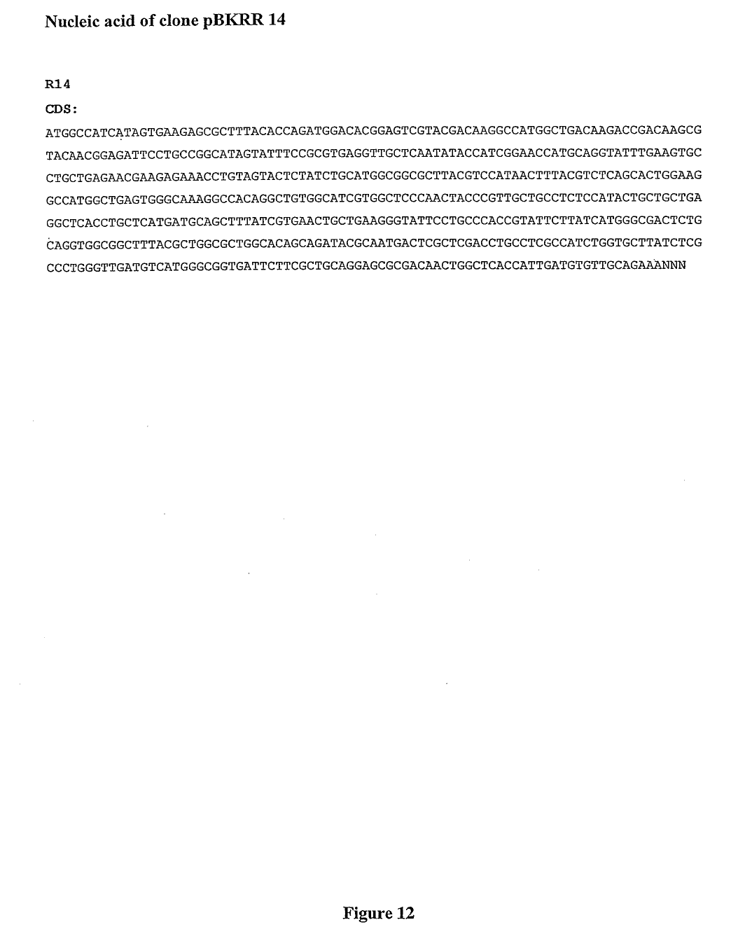

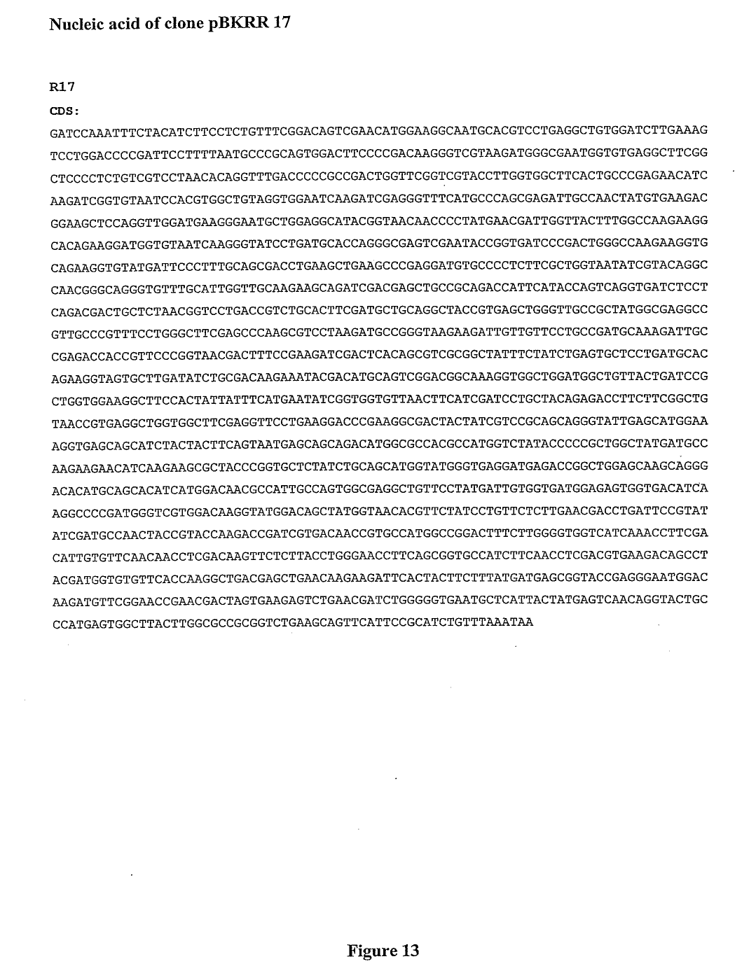

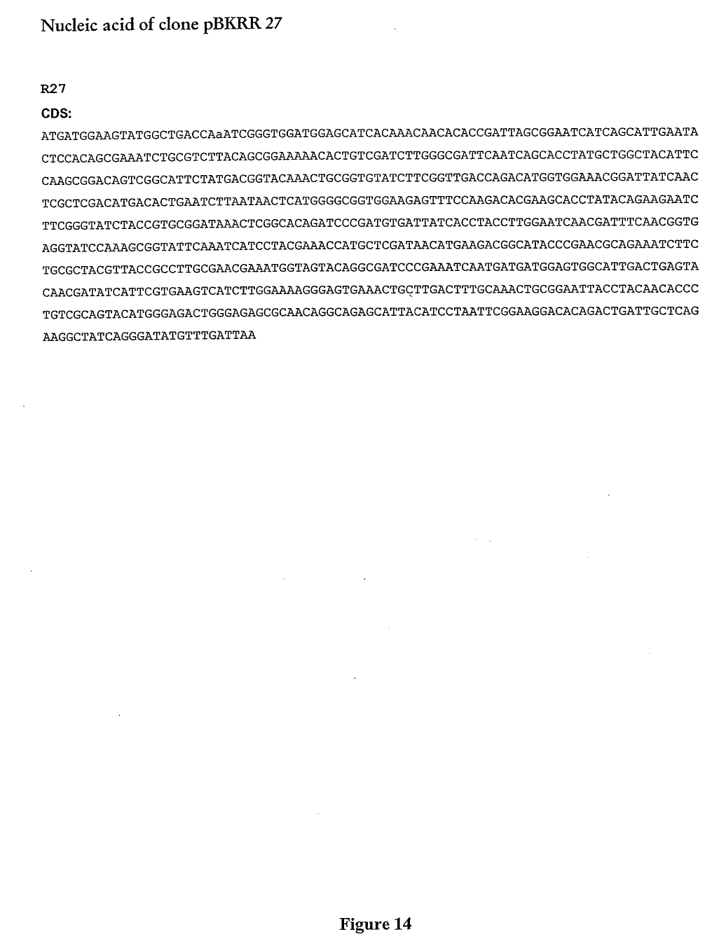

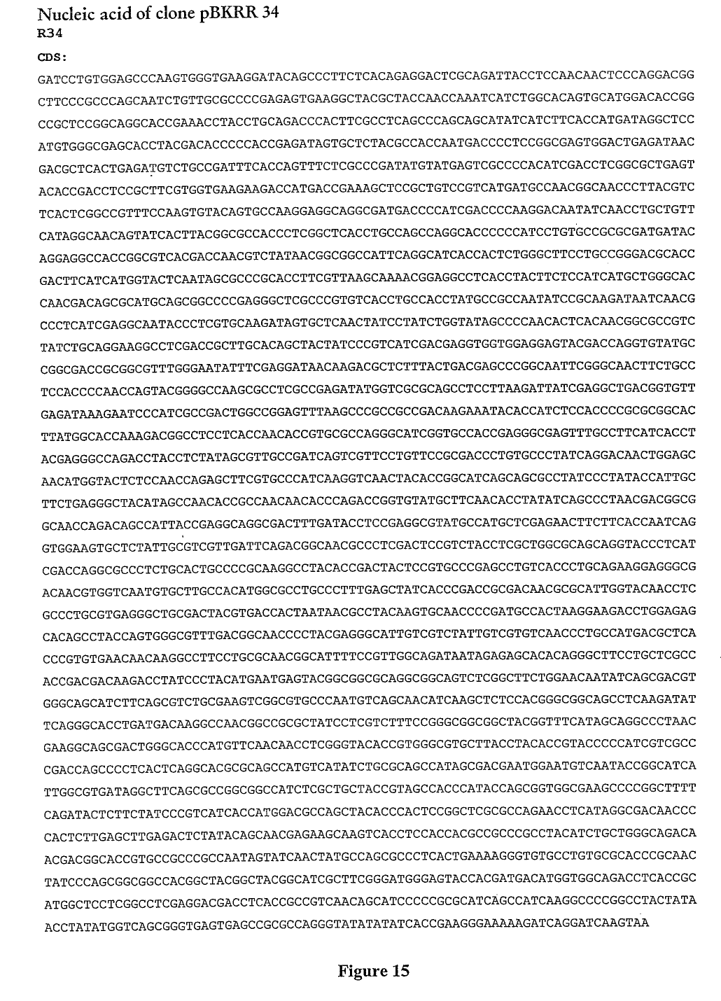

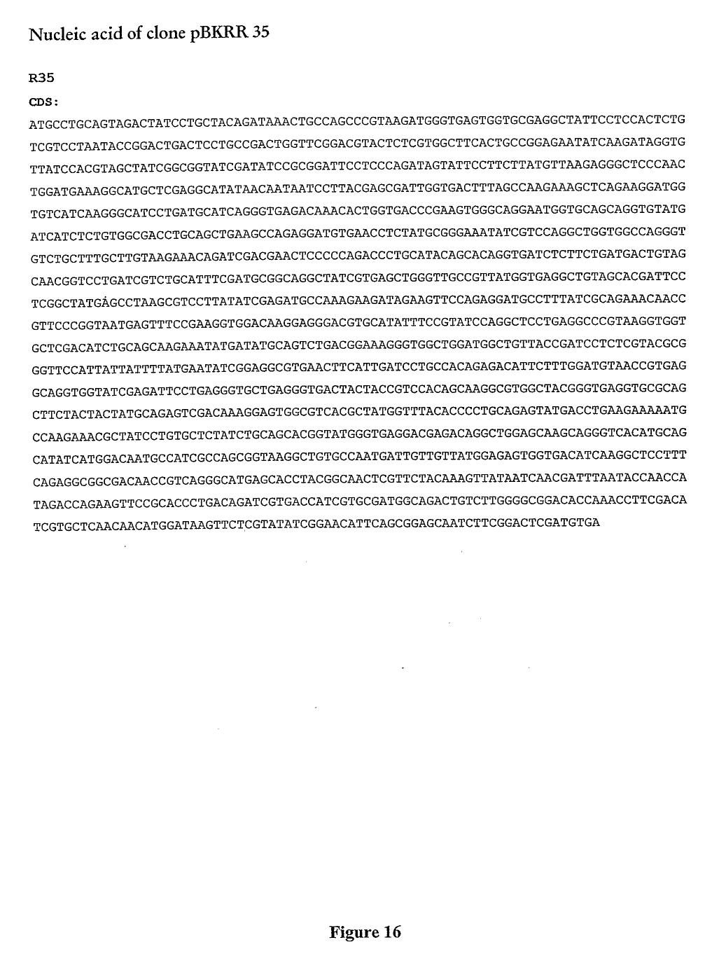

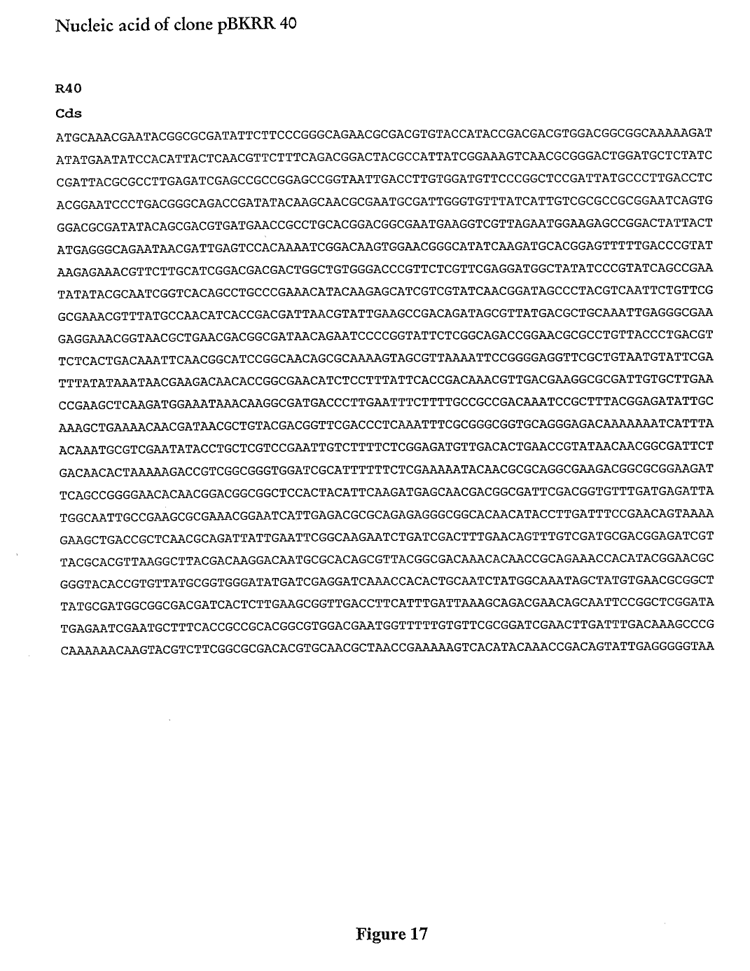

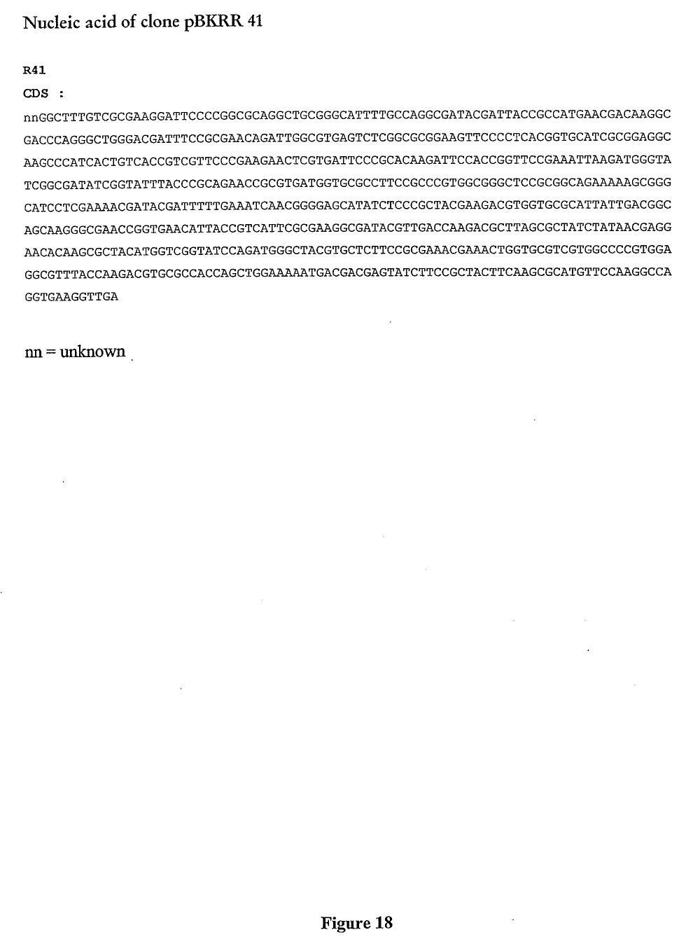

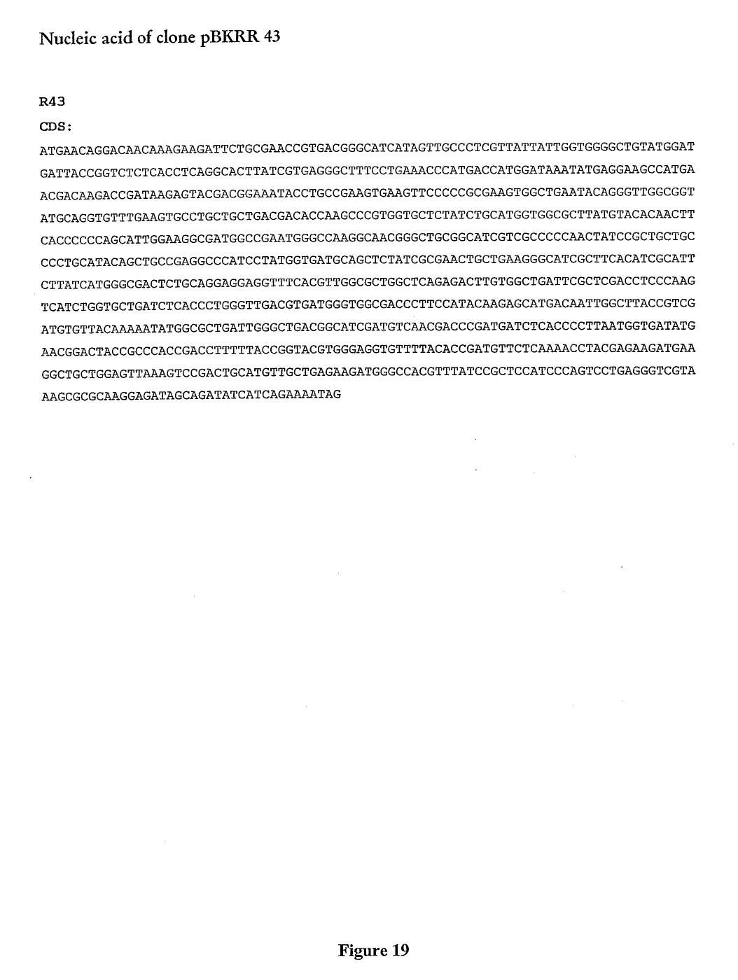

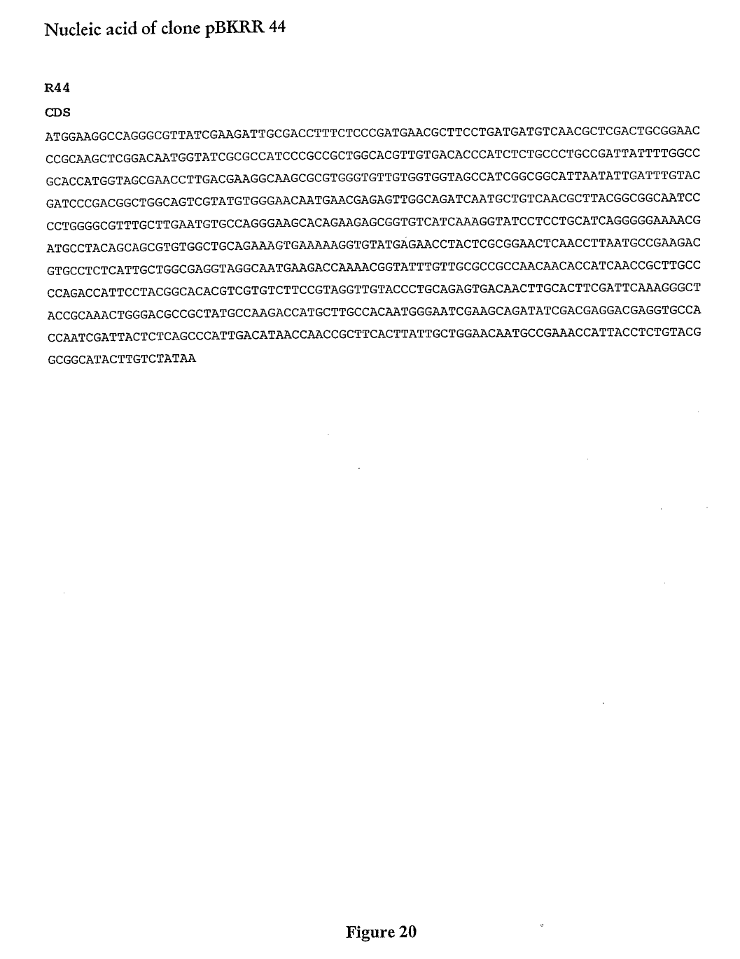

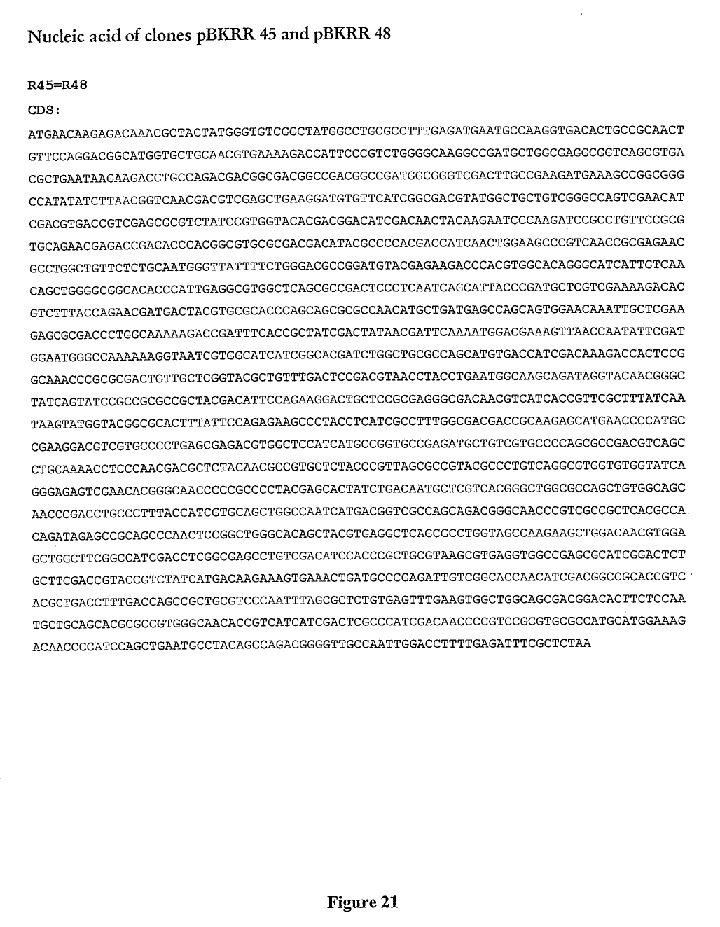

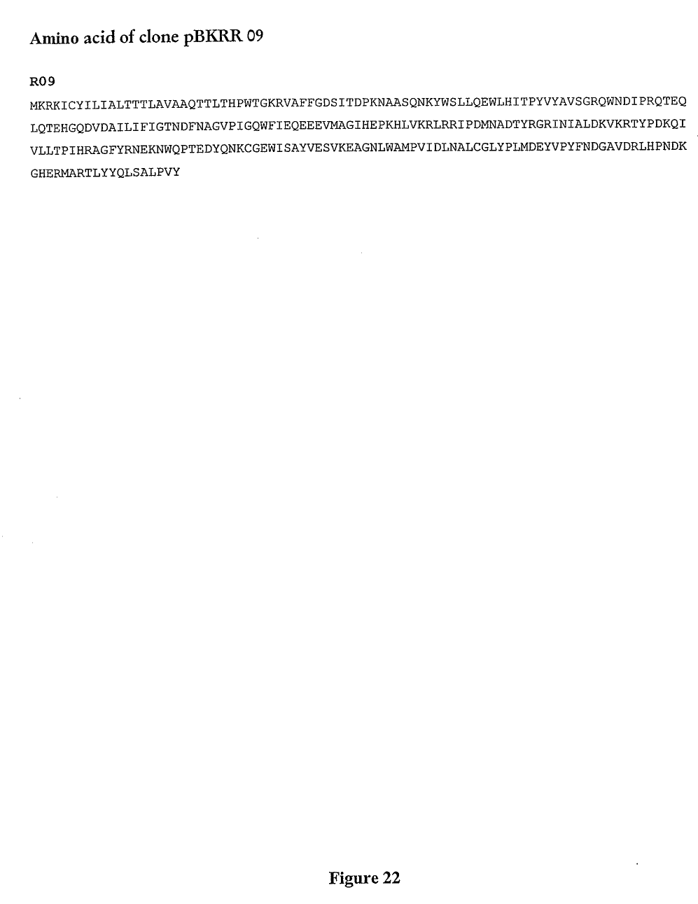

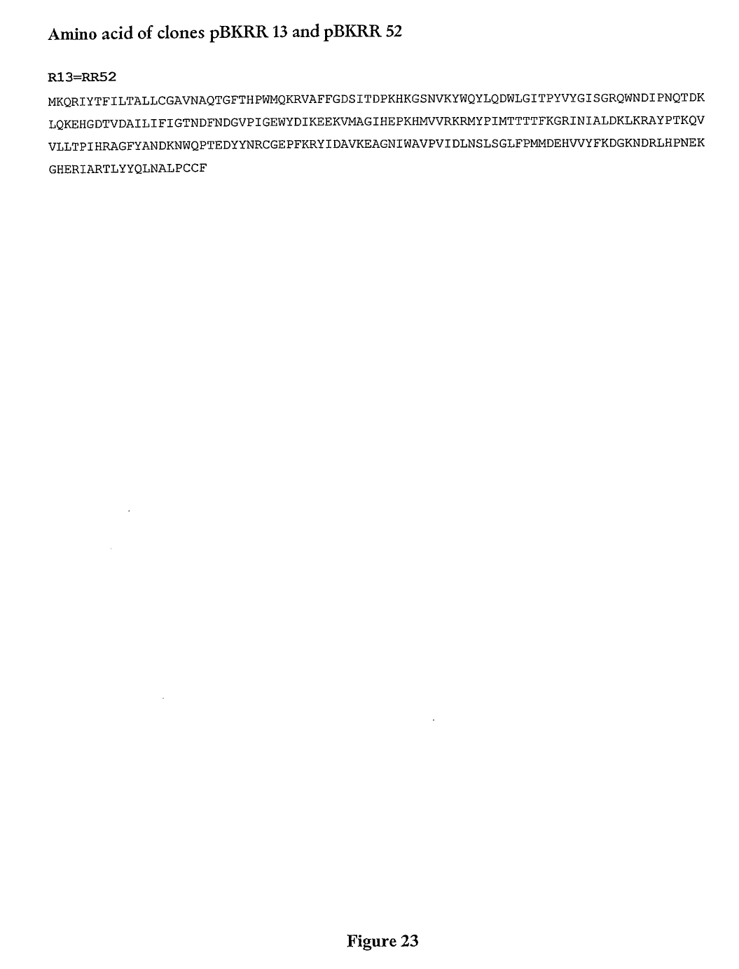

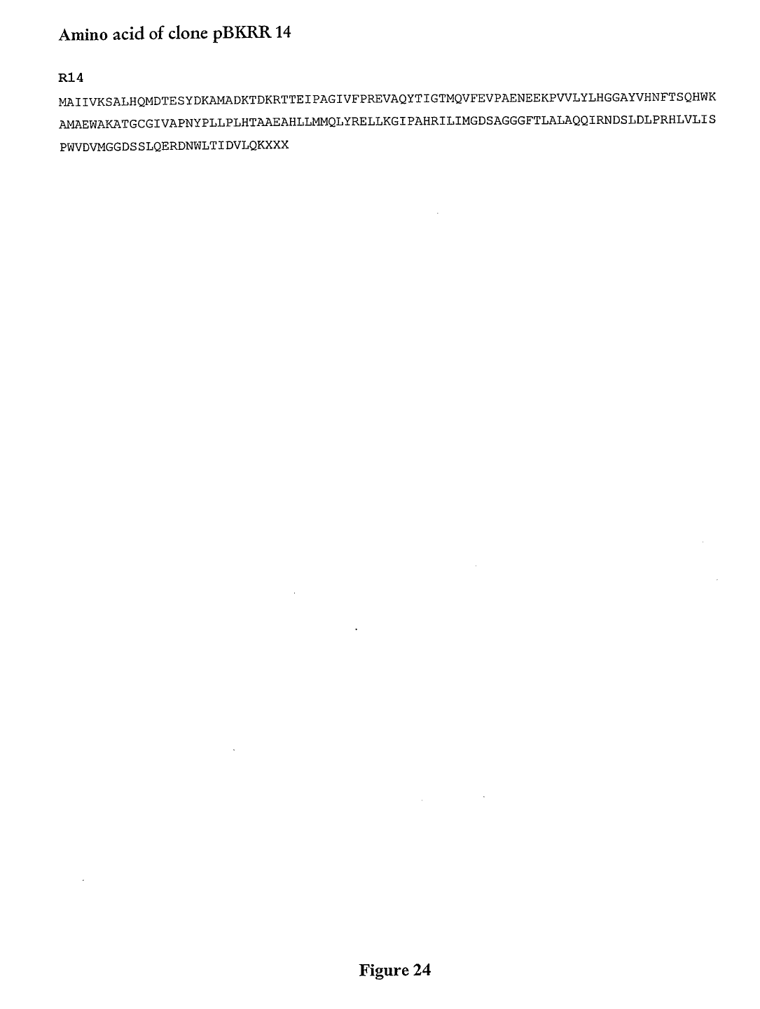

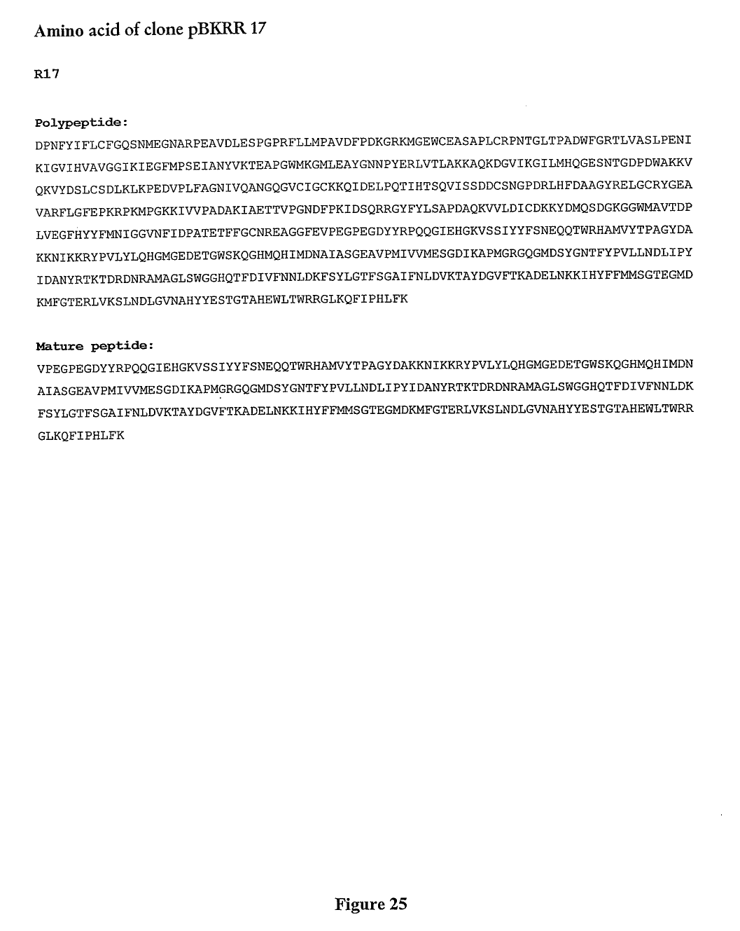

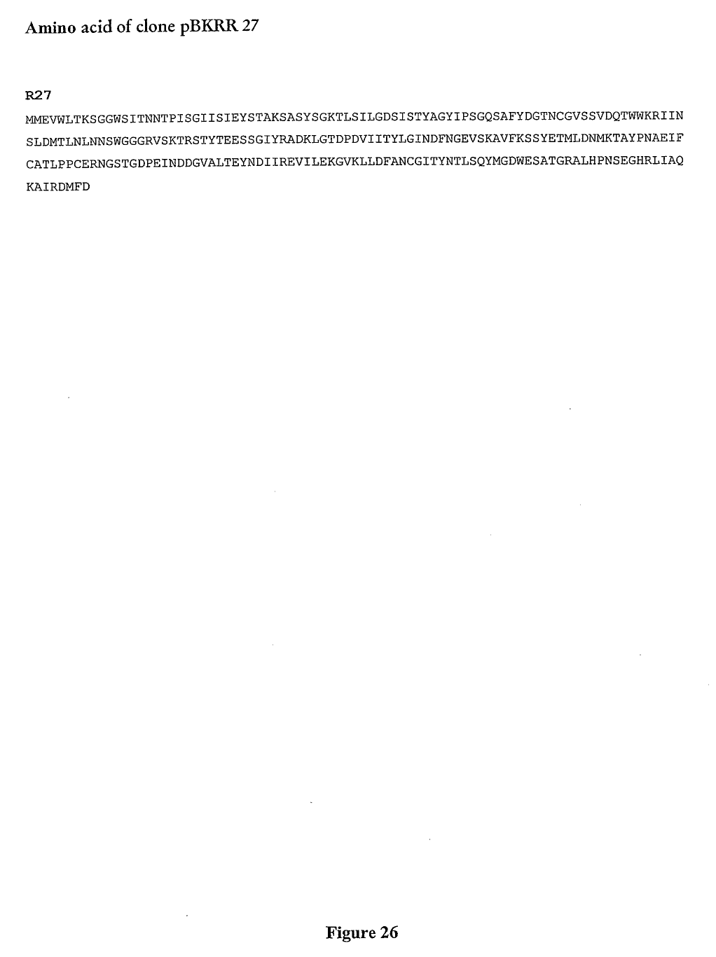

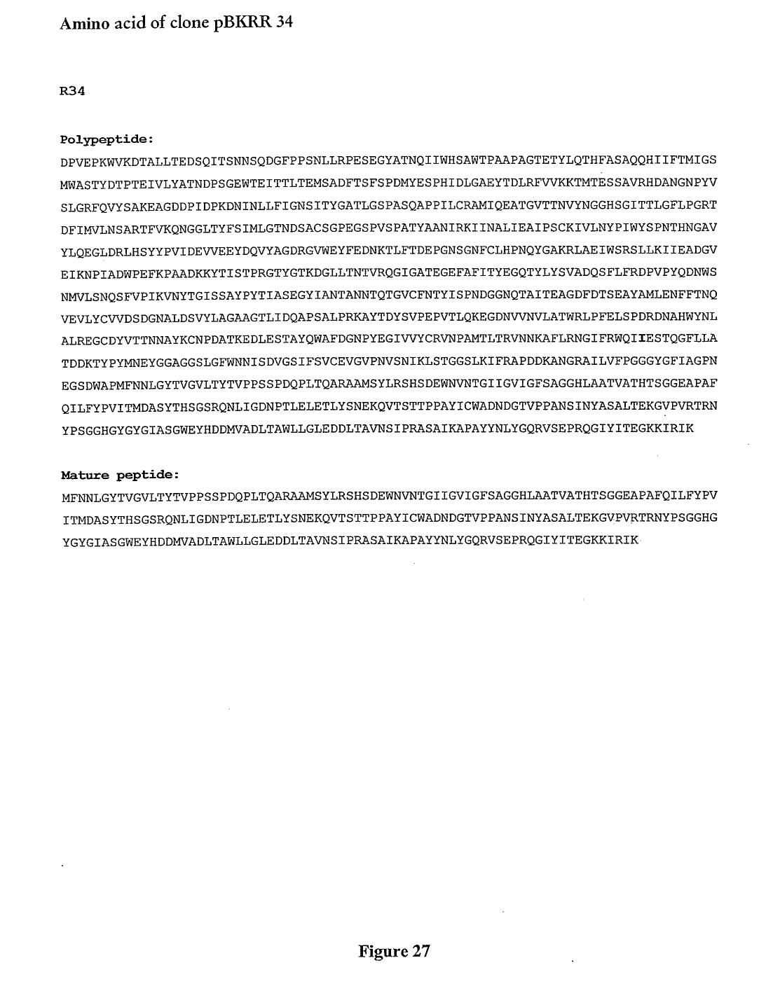

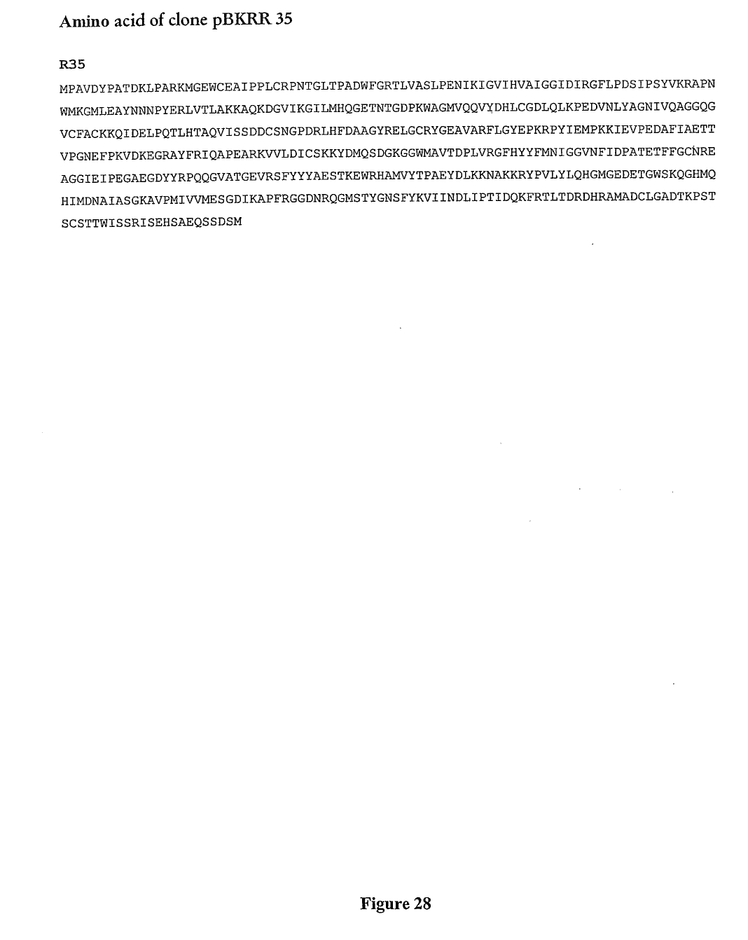

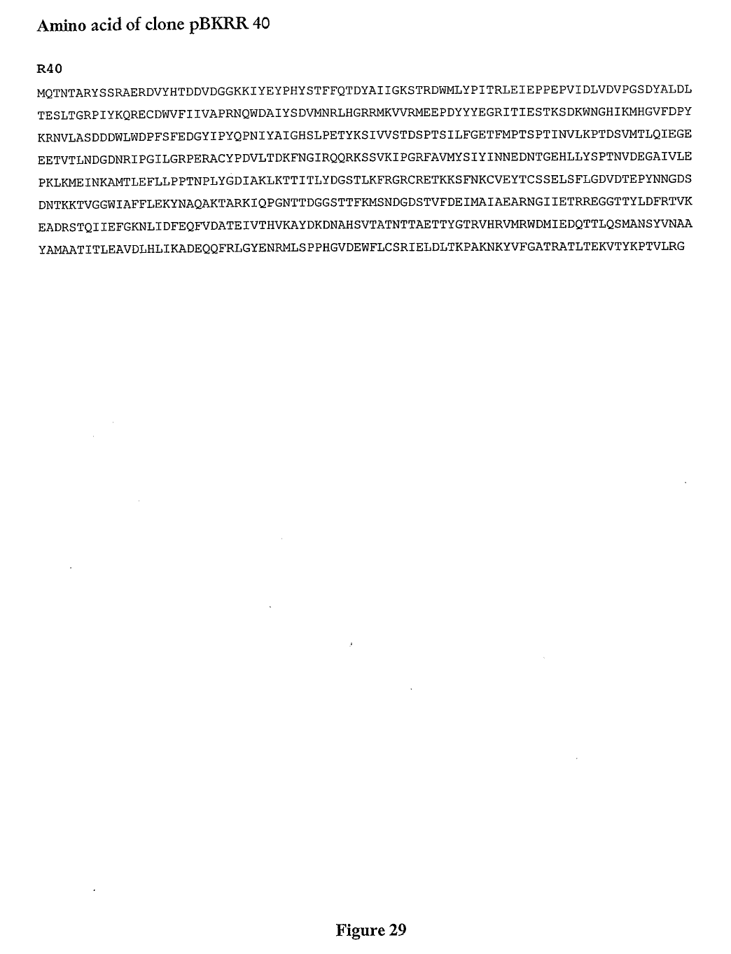

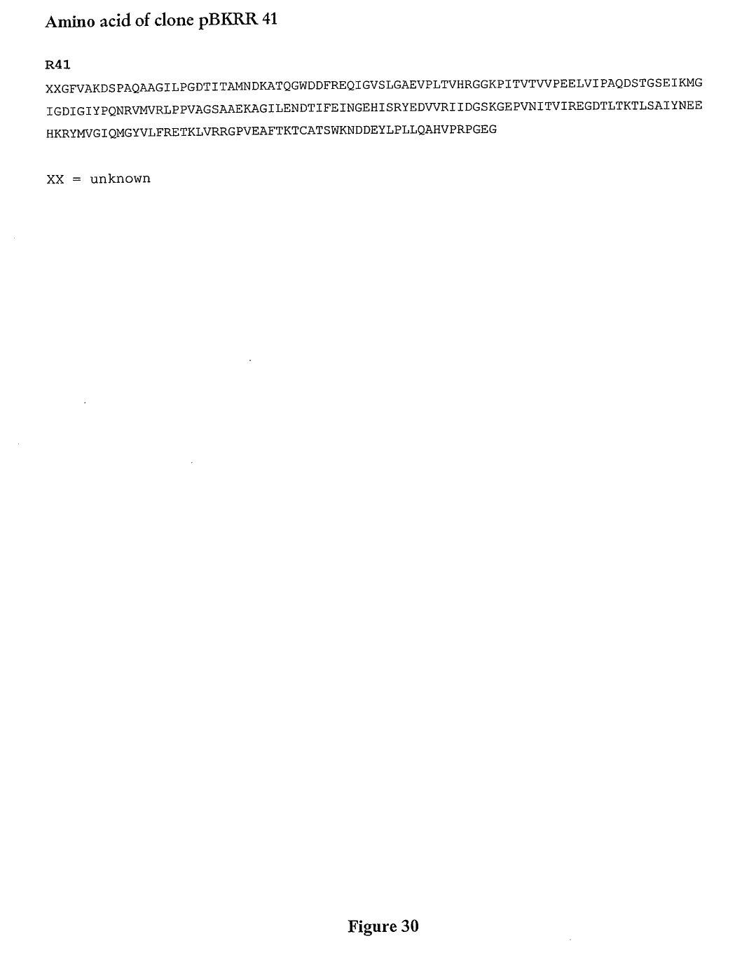

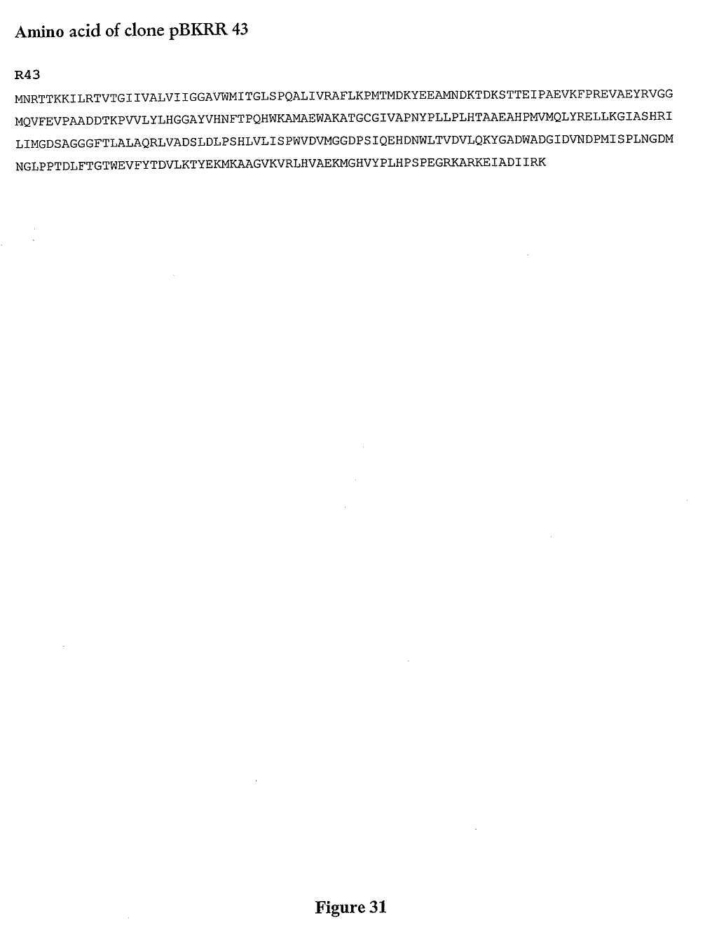

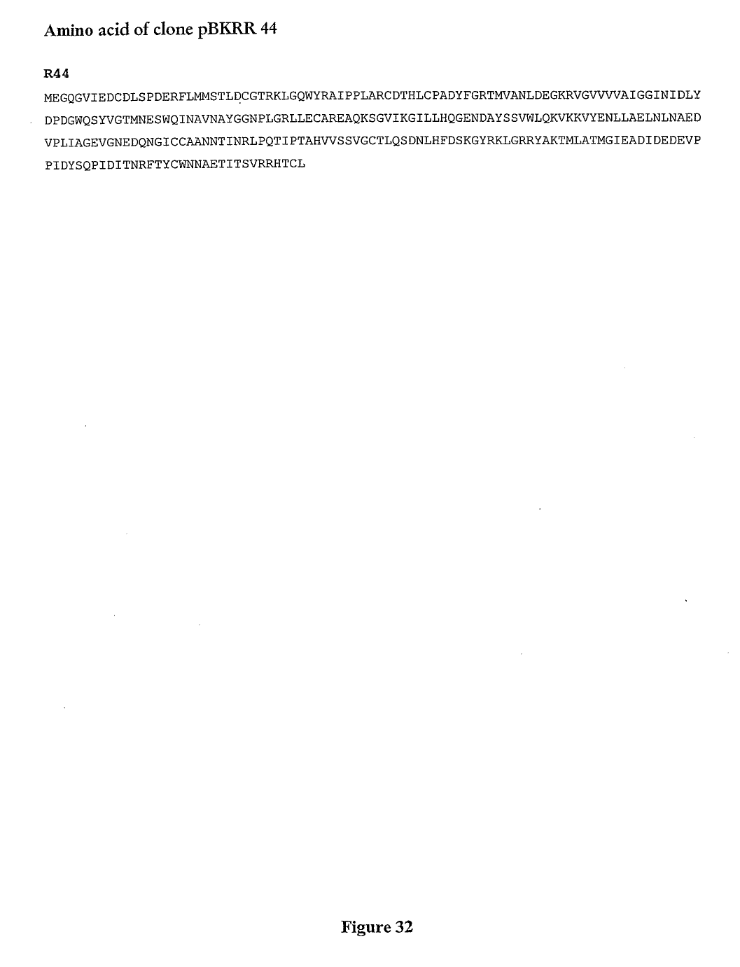

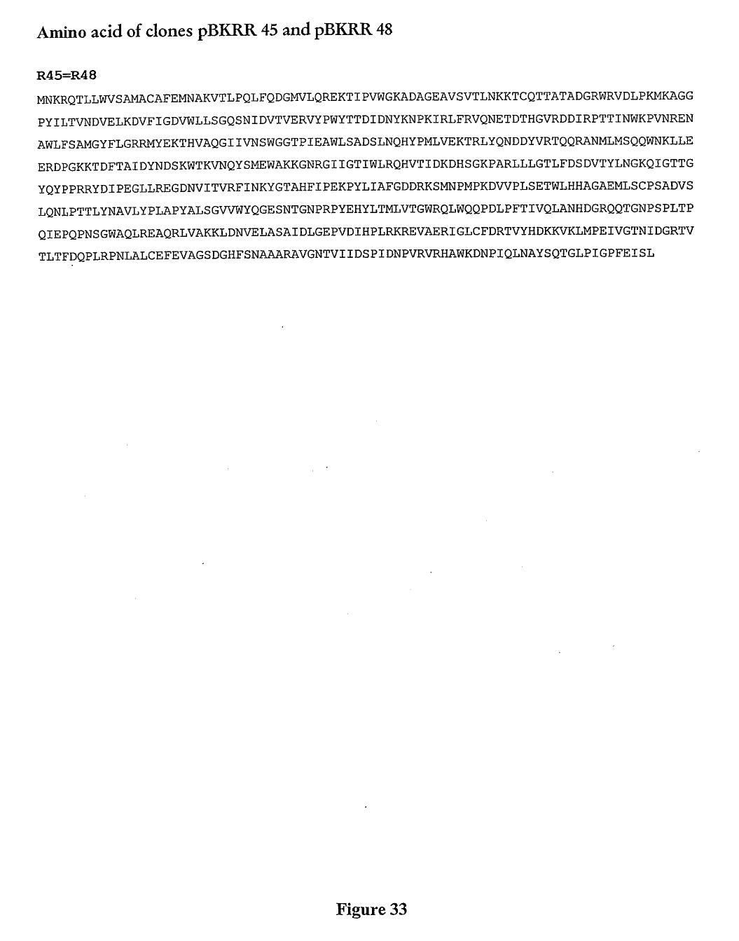

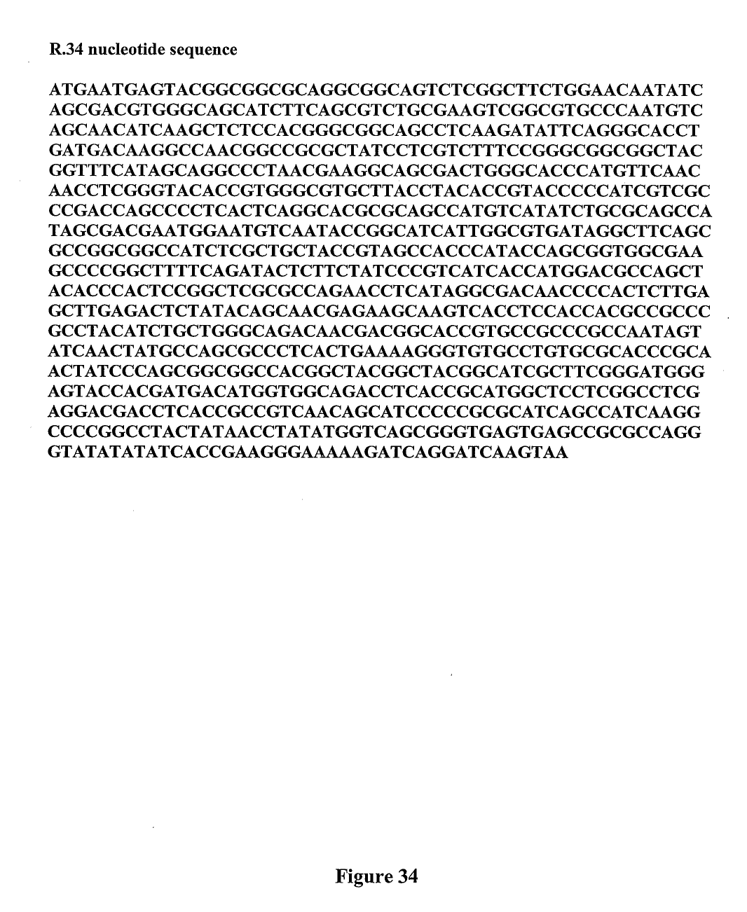

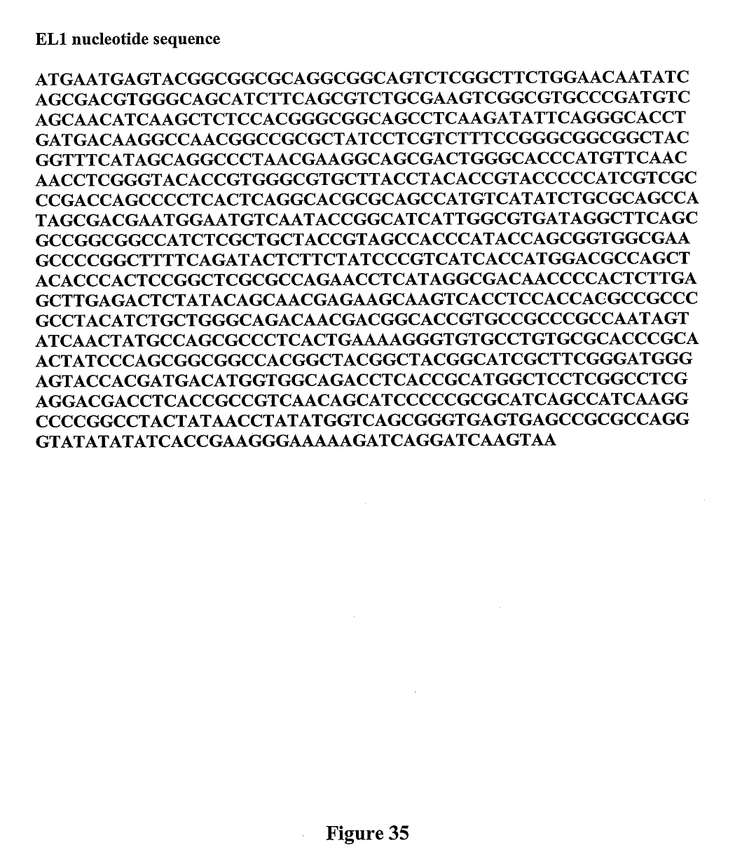

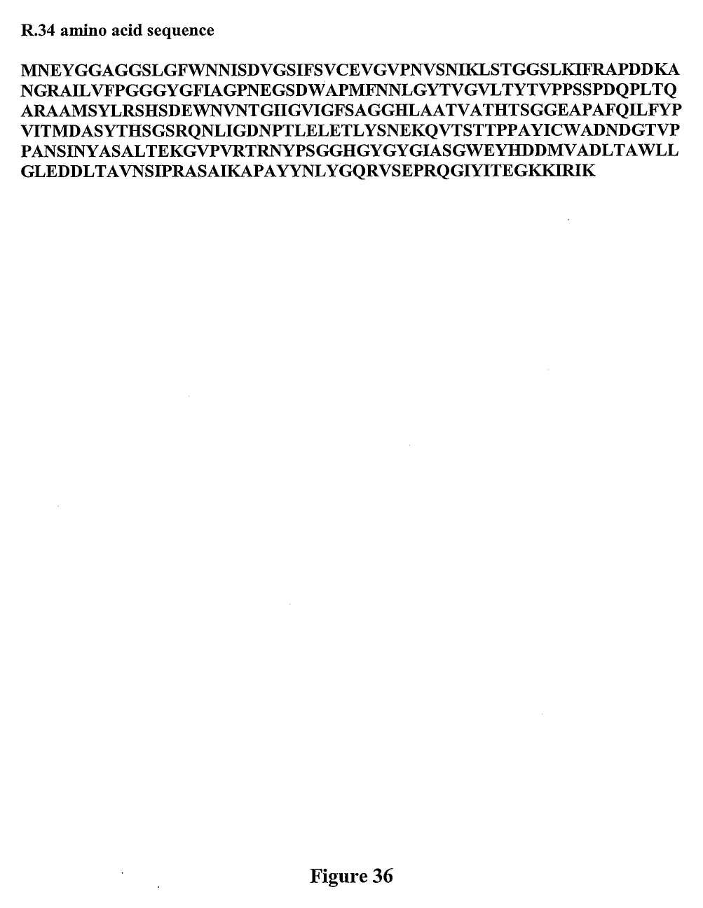

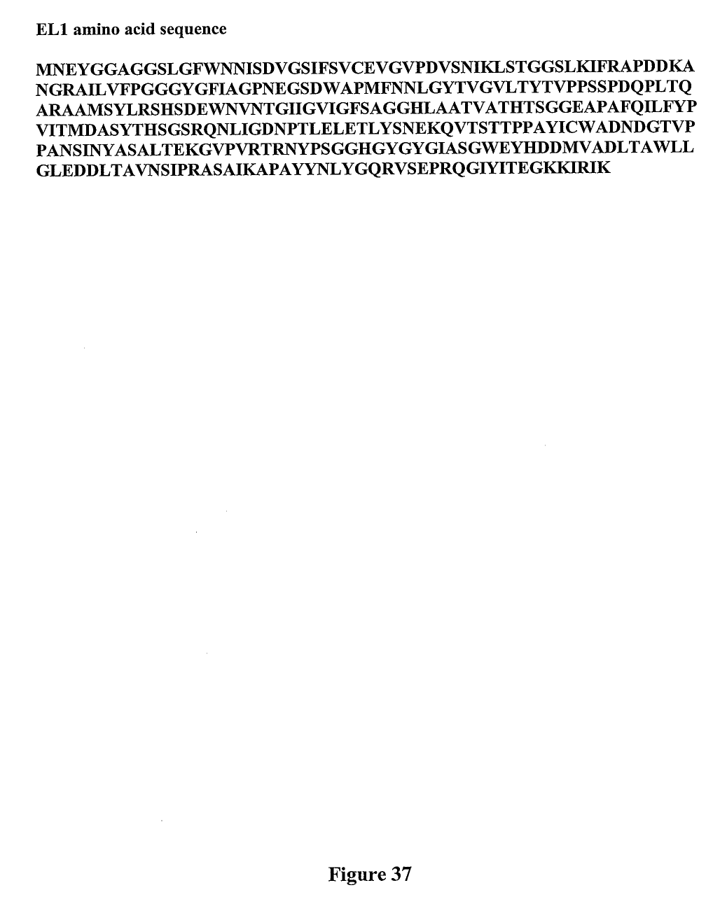

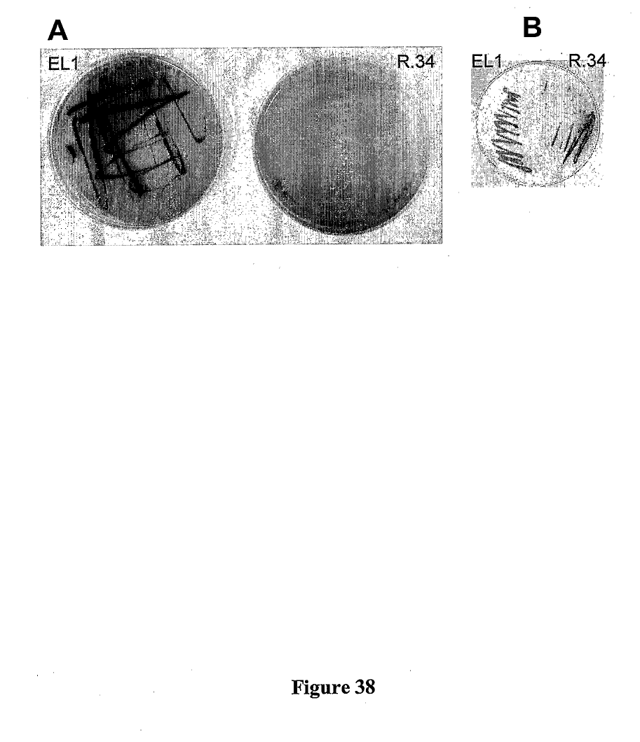

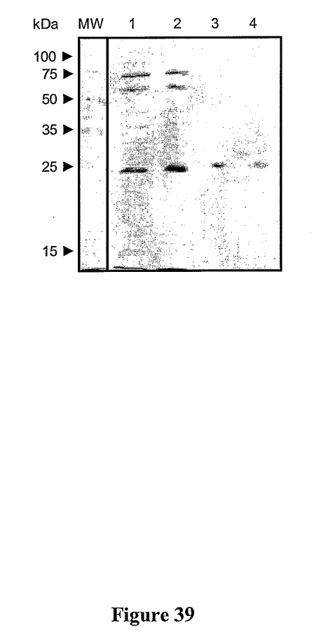

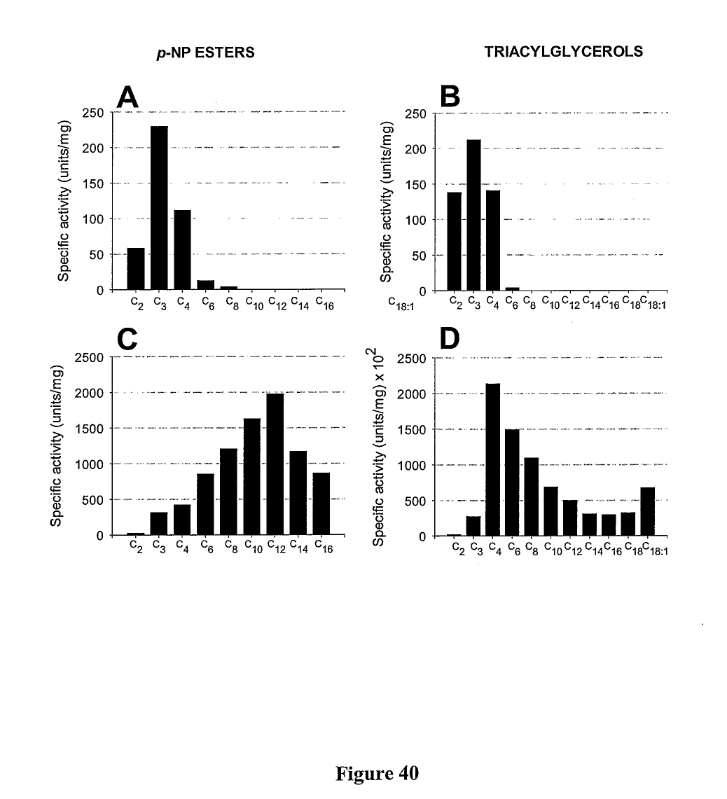

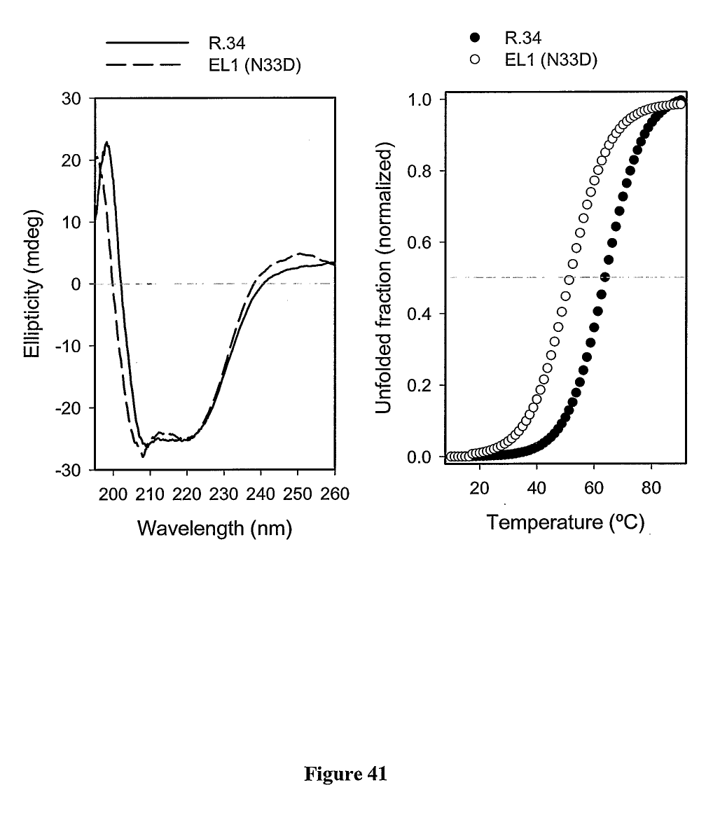

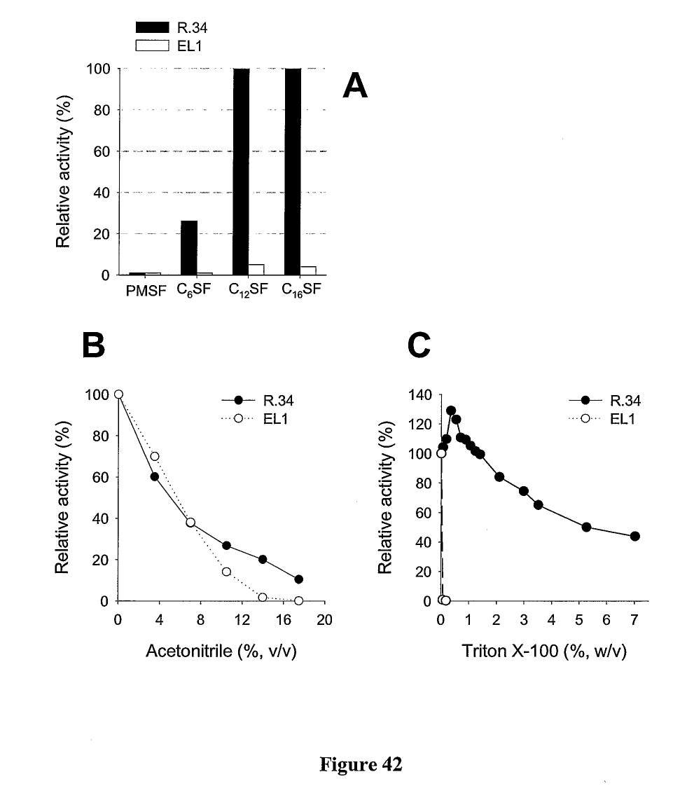

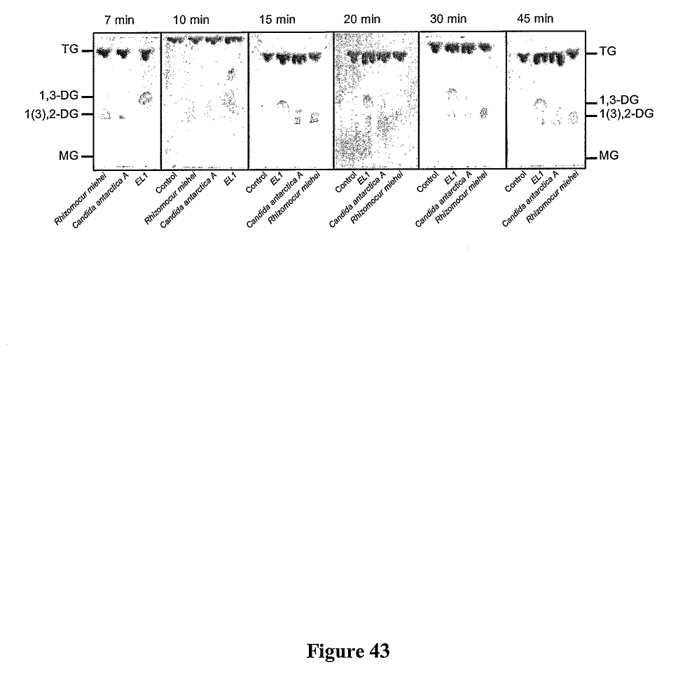

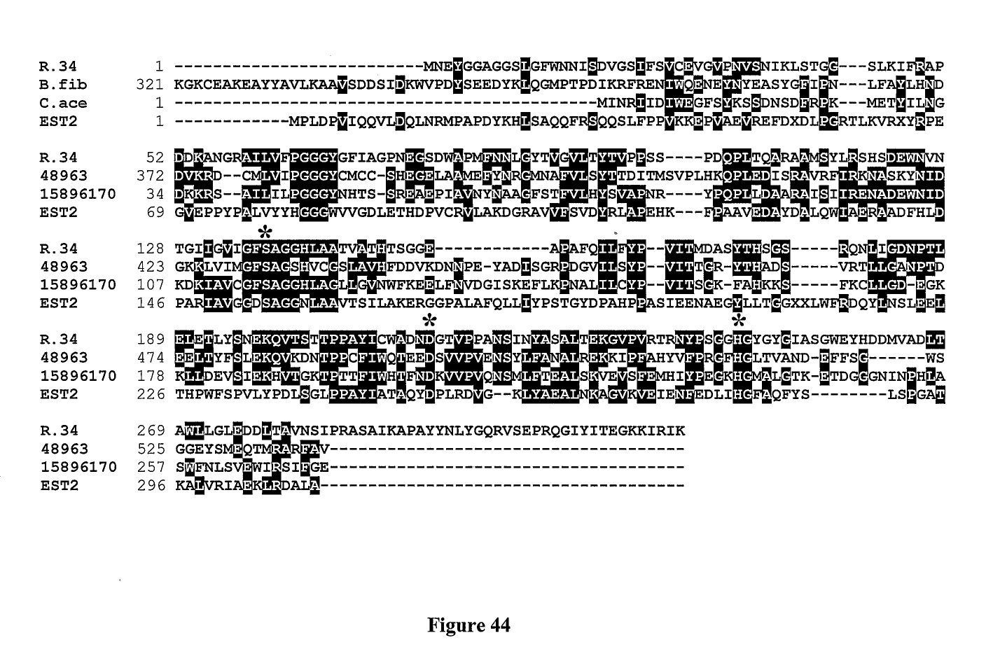

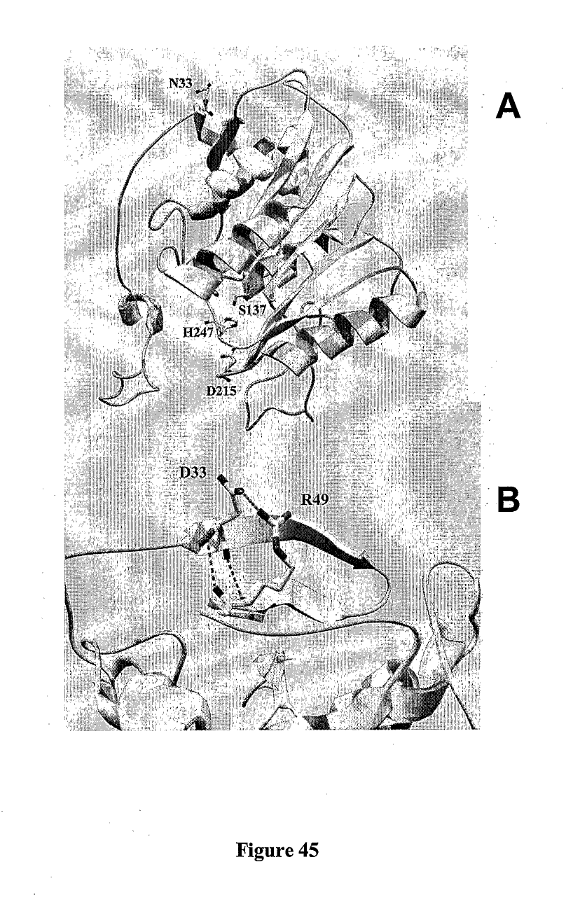

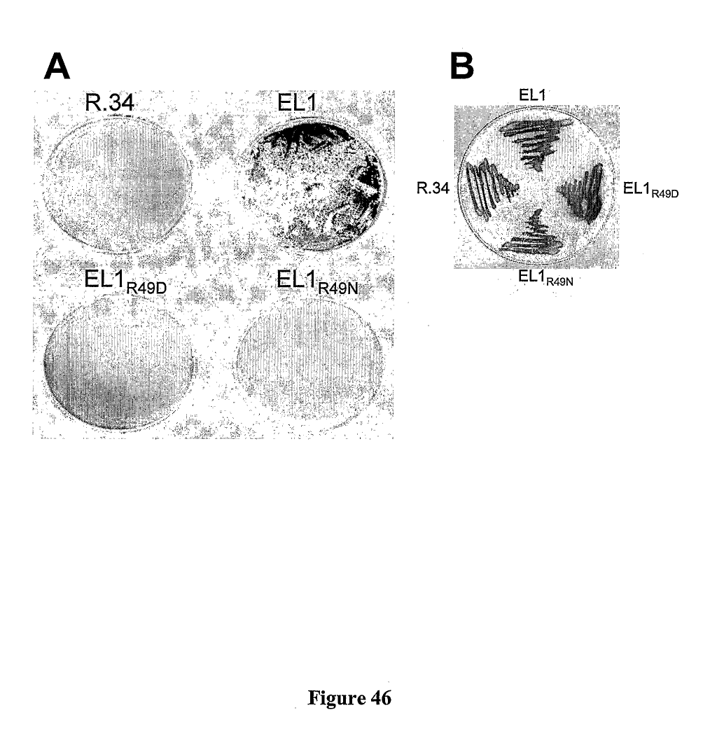

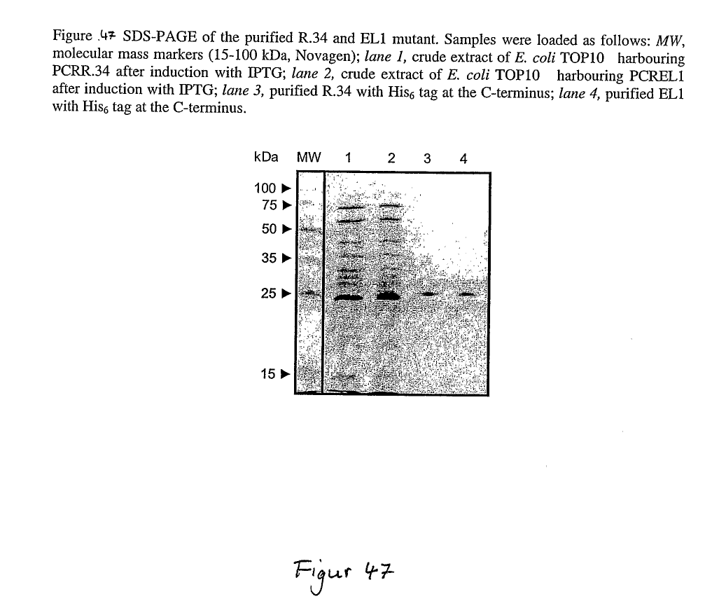

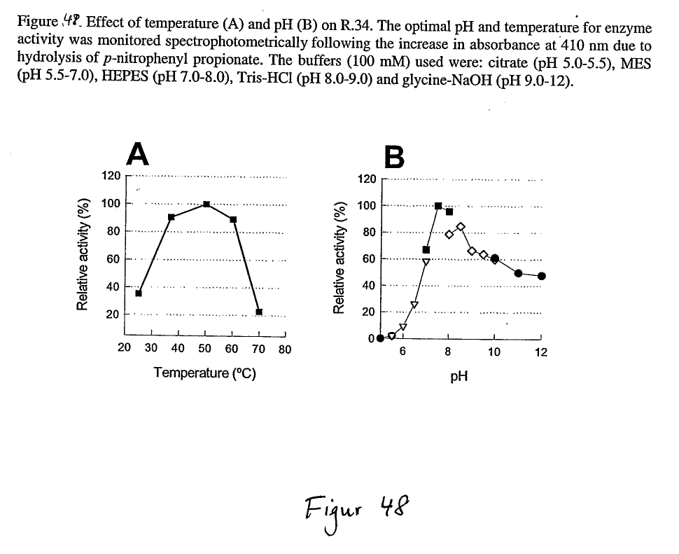

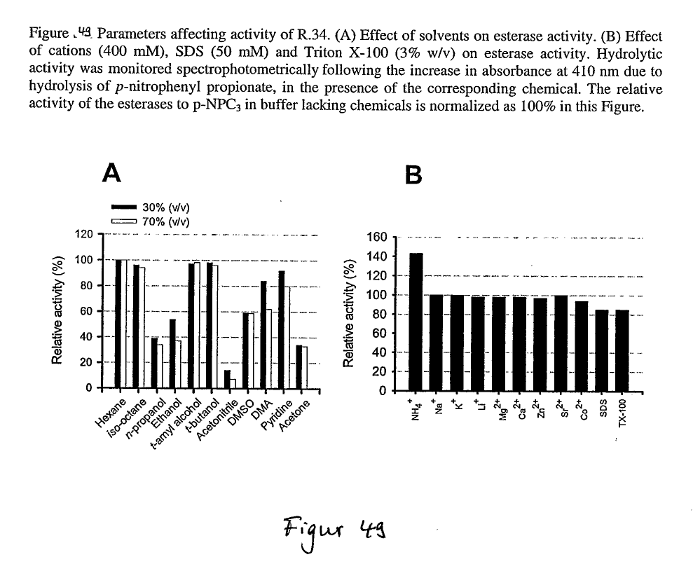

Acyl chain specificity: Substrate specificity of purified R.34 was determined using p-NP esters and triacylglycerols of varying chain length. Higher activity of R.34 was shown towards p-NP propionate (C₃) (230 units/mg) as substrate (see main text, FIG. 1A). the activity of the enzyme decreased 2-fold with p-NP butylate (C₄), and was very low for p-NP caproate (C₆). R.34 was also able to hydrolyze triacylglycerols shorter than C₄ (optimum with tripropionin: 210 units/mg). p_NP esters and triacylglycerols with acyl chains longer than C₄, triolein and olive oil were poorly (or not) hydrolyzed, which suggests that the enzyme is an esterase rather than a lipase. In order to exclude any influence of the His₆ tag on the recombinant esterase activity, the protein was expressed in an purified from E. coli XLOLR cells harboring pBKR.34 plasmid, by native gel electrophoresis and gel filtration chromatography as will be described in elsewhere (Ferrer et al., unpublished). Enzyme obtained by this method did not show relevant differences in substrate specificity (Table S2).

Optimal temperature and pH: R.34 showed maximum activity at 50° C. although enzyme activity was retained over a temperature range from 35 to 60° C. with activity falling drastically beyond this range (FIG. 48A). Esterase activity was most active at pH 7.5-8.0, although it retains more than 50% activity at an alkaline pH range 9.0-12.0 (FIG. 48B).

Enantioselectivity of R.34 esterase: We employed the Quick E colorimetric assay (L. E. Janes, C. Löwendahl, and R. J. Kazlauskas, Chem. Eur. J. 1998, 4, 2317-2324) to analyze the enantioselectivity of R.34 esterase. Firstly, we assayed a variety of racemic esters to eliminate substrates that were not hydrolyzed (Table S3). This screening identified four potential racemic substances (specific activity over 570 units/mg). We then estimated the enantioselectivity (Eₐₚₚ) of the hydrolase by separate measurements of the initial rates of hydrolysis of each enantiomer using the Quick E assay. It should be mentioned that the ratios obtained by these measurements were not true enantiomeric ratios (Eₜᵣᵤₑ), because the rates of hydrolysis of the enantiomers were measured separately; nevertheless, recent studies have clearly demonstrated that apparent (Eₐₚₚ) and true (Eₜᵣᵤₑ) enantioselectivity values closely match each other (U. T. Bornscheuer. Eng. Life Sci. 2004, 4, 539-542). As shown, R.34 esterase exhibited good enantiomeric ratios (Eₐₚₚ values of 18.5-117; Table S4), although enantiopreferences varied with substrate.

Influence of chemicals: The effect of different solvents, metal ions and detergents on R.34 activity was determined. The level of activity of the enzyme after 24 h incubation with each chemical did not display significant differences from the activity observed when the activity was measured immediately. It is well known that organic solvents affect the enzyme activities of different lipases and esterases, which are different from each others. The purified esterase was incubated with various water-miscible and immiscible solvents at 40° C. A concentration range from 30 to 70% v/v was used. As shown in FIG. 49A, esterase was active and stable in non-polar solvents such as hexane, iso-octane and pyridine, medium polar solvents such as tert-butyl and tert-amyl alcohol, as well as polar solvents such as dimethyl sulfoxide and dimethyl acetamide, normally used in biocatalysis. The purified enzyme exhibited hydrolytic activity without addition of any metal ion (FIG. 49B). Any of the cations tested inhibited R.34 under the experimental conditions tested (400 mM) although it was slightly activated by NH₄⁺ (1.4-fold). Moreover, the addition of ethylenediaminetetraacetic acid (EDTA) or ethyleneglycoltetraacetic acid (EGTA) did not result in a decrease in esterase activity, indicating that esterase functioning was independent of divalent cations. We further observed that R.34 was resistant to high surfactant concentration (50 mM SDS and 3% w/v Triton X-100) (FIG. 49B), agents that rapidly inactivate most esterases and lipases.

It was found that R.34 has a typical sequence motif (GDS(L)) being typical for family II ester hydrolases (FIG. 47). R34 is a monomeric protein of Mr 25.810 Da and an isoelectric point of 4.57. It is an esterase hydrolyzing esters with fatty acid chains of 4 or less carbon atoms, ideally 3 carbon atoms. Its maximum activity was found to be at 50° C. and pH 7.5-7.8 (FIG. 48). R.34 showed excellent enantioselectivities by using the Quick E colorimetric assay. R.34 was stable and active in non-polar solvents, such as hexane and pyridine, or medium polar solvents, like tert-amyl alcohol, or polar solvents, like DMSO. It exhibited hydrolytic activity without adding metal ions. The activity was maintained even in the presence of surfactants at high concentrations (50 mM SDS or 3% w/v Triton X-100), agents which normally inactivate most esterases and lipases (FIG. 49). The results are summarized in Tables S1 to S4.

The esterase R34, the amino acid and nucleic sequence of which is disclosed and claimed herein in the same way as the other sequences disclosed herein, was used as starting material for the identification of the second invention described in the following.

Similarly to esterases, lipases (the second invention herein) represent a group of enzymes with increasing importance for classical and new industrial applications as described above. The most significant properties of both are that they are very stable and active, even in organic solvents, and possess regio- and stereo-specificity. Despite their close relationship in sequence and structure, these enzymes show relevant differences in their profile for chain length specificity. While esterases (EC 3.1.1.1) hydrolyze preferentially esters solely soluble in water (mostly triglycerides with short-chain fatty acids, in general shorter than C₆ [C₆ means 6 carbon atoms]), lipases (EC 3.1.1.3) prefer water-insoluble substrates, typically triglycerides composed of long-chain fatty acids (U. T. Bornscheuer, FEMS Microbiol. Rev. 2002, 26, 73-81). Thus, clear experimental evidence to distinguish between esterase and lipase activity is the determination of their ability to hydrolyze long-chain acyl glycerols (Verger, R., Trends Biotechnol 1997, 15, 32-38).

Lipases were defined in kinetic terms based on the phenomenon of interfacial activation (Sadra L. et al., Biochim. Biophys. Acta 1958, 30, 513-521). It amounts to the fact that the activity of lipases is low on monomeric substrates but becomes strongly enhanced once an aggregated “supersubstrate”, for example an emulsion or a micellar solution, is formed above its saturation limit. This property is quite different from that of esterases acting on water-soluble carboxylic ester molecules. Therefore, a distinct feature of lipases compared to esterases is that they catalyze ester hydrolysis at the lipid-water interface in spite of their water solubility.

In addition, lipases can accommodate a wide range of substrates other than triglycerides (having three ester bonds coupling glycerol to fatty acids). E.g. lipases may catalyze reactions involving substrates with less than three ester bonds, in particular substrates containing just one ester bond which may comprise aliphatic, alicyclic, bicyclic and aromatic esters and even esters based on organometallic sandwich compounds. Lipase substrates having one or two ester bonds are designated in the following mono- or diacylester. With respect to racemic esters or substrates with several hydroxyl groups, lipases react with high enantio- and regioselectivity (Chen et al., Angew. Chem. 1998, 101, 711-724; Angew. Chem. Int. Ed. 1998, 28, 695-708). Finally, the acyl enzyme intermediate in lipase-catalyzed reactions is not only formed from carboxylic esters but also from a wide range of other substrates such as thioesters or activated amines, which increases the synthetic potential of lipases considerably (Gutman, A. L. et al., Synthetic Applications of Enzymatic Reactions in Organic Solvents, Adv. Biochem. Eng. Biotechnol., Vol. 52 (Ed. Fiechter), Springer Heidelberg, 1995, 87-128). In summary, the broad application potential of lipases is largely due to the fact that lipases, contrary to esterases and most other enzymes, accept a wide range of substrates, are stable in non-aqueous organic solvents, and thus, depending on the solvent system used, can be applied to ester synthesis as well as hydrolysis reactions (Schmid, R. D. et al., Angew. Chem. Int. Ed. 1998, 37, 1608-1633).

With respect to triglycerides (=esters of triglycerols), lipases may hydrolyze their primary (sn-1 position), secondary (sn-2 position) and/or tertiary (sn-3 position) ester bonds. While most of the lipases hydrolyze sn-1 and/or sn-3 ester bonds, only few lipases are able to hydrolyze sn-2 ester bonds. Rogalska et al. (Chirality 1993, 5, 24-30) reported that Candida rugosa lipase (CR), Pseudomonas glumae lipase (PG), Candida antarctica A lipase (CAA), Fusarium solani cutinase (FSC) and Penicillium simplississimum lipase (PS) were the only lipases capable of hydrolyzing sn-2 ester bonds from trioctanoyl and trioleoylglycerol. These five lipases were selected for further investigations concerning the hydrolysis of sn-2 ester bonds. However, no lipase acting exclusively sn-2 specific is yet known in the art (Douchet, I. et al., Chirality 2003, 15, 220-226). In fact, all above mentioned at the sn-2 position hydrolyzing lipases are involved in the hydrolysis at the sn-1 position and/or sn-3 position of triglycerides as well converting triglycerides to free fatty acid and glycerol. Thus, no enzymes are known in the art, which hydrolyze ester bonds sn-2 specifically. Furthermore, even though PG hydrolyzes preferably (but not specifically) sn-2 ester bonds, it shows strong substrate specificity, namely for specific triglycerides. However, it is desired to have polypeptides showing sn-2 specifity combined with catalytic activity for a wide range of substrates, which include any triglyceride substrate as well as any mono- and diacylester substrate.

In summary, industrial applications are restricted to the use of 1-specific, 3-specific or 1,3-specific lipases to provide products of the hydrolytic or esterizing reaction, which may be used e.g. as nutritional lipids. Provision of a highly sn-2 specific lipase would allow the enzymatic synthesis of lipid products not yet obtained by state-of-the-art lipases. Therefore, there is also need for lipases hydrolyzing preferably or exclusively the sn-2 position of triglycerides combined with hydrolyzing activity for a broad range of substrates including mono- and diacylesters.

Thus, it is an object of the present invention to provide an enzymatic system for hydrolyzing lipase substrates characterized by long-chain fatty acids, and, furthermore, for hydrolyzing preferably or exclusively ester bonds at the sn-2 position of triglycerides.

This technical problem is solved by the second invention relating to a polypeptide comprising the amino acid sequence of amino acids No. 20 to No. 50 of the amino acid sequence shown in FIG. 37 or a functional fragment, or functional derivative thereof. Preferably, the polypeptide comprises the amino acid sequence of amino acids No. preferably No. 18 to No. 70, more preferably No. 15 to No. 100, most preferably No. 10 to No. 130 of the amino acid sequence shown in FIG. 37. Most preferably, the polypeptide comprises the amino acid sequence shown in FIG. 37. Amino acid No. 33 of the sequence shown in FIG. 37 is characterized by a characteristic binding and/or catalytic site, which is different from all known prior art lipase enzymes hydrolyzing ester substrates. The characteristic feature is the substitution of Asn by Asp (N33D) leading to a formation of an additional ionic pair (for further details see below, especially specification of FIG. 45). Thereby, the inventive polypeptide may bind and catalyze ester bond substrates having long-chain fatty acids in contrast to the original sequence characterized by Asn at amino acid No. 33 (of FIG. 37).

The present invention is based on the discovery that specific techniques, especially the so-called directed evolution technique, can lead to improved enzyme properties, like thermal stability (Zhang, N. et al, Protein Eng. 2003, 16, 599-605; Acharya, P., J. Mol. Biol. 2004, 341, 1271-1281, Santarossa, G., FEBS Lett. 37 2005, 579, 2383-2386), specificity, substrate selectivity, inverted or improved enantioselectivity (Bornscheuer, U. T., Biotechnol. Bioeng. 1998, 58, 554-1 4-559; Krebsfänger, N, J. Biotechnol. 1998a, 60, 105-111; Liebeton, K., 2000. Chem. Biol. 7, 709-718) and altered activity. According to the invention, directed evolution was used to convert an naturally occurring esterase (being disclosed according to the first invention of the present invention) as starting material into an enzyme having lipase activity.

According to the invention the short-chain specific and chemically stable esterase R.34 (which corresponds to pBKR.34 described above by the first invention) from rumen metagenome was converted into an enzyme exhibiting lipase activity (polypeptide of FIG. 37, in the following also designated as EL1). The inventive lipase EL1 according to the invention exhibited a complete change of substrate specificity compared to esterase R.34. Preferably, p-NP esters (t-nitrophenyl esters; substrate belonging to the class of mono- and diacylester) and triglycerides were used as test substrates. Whereas esterase R.34 hydrolyzes short-chain p-NP esters from C₂ to C₆ carbon atoms, with an optimum for C₄, inventive lipase EL1 hydrolyzes mainly long-chain p-NP esters from C₆ to C₁₆. Furthermore, in contrast to R.34, which hydrolyzes short-chain triglycerides having fatty acid chains of up to C₄, lipase EL1 hydrolyzes short-chain triglycerides as well as long-chain triglycerides up to C₁₈. Therefore, the present invention provides a substrate-specific lipase characterized by its enzymatic preference for monoacylesters with fatty acid chains of more than C₄ and triglycerides with fatty acid chains having more than C₆.

Preferably, the inventive polypeptide having lipase activity hydrolyzes substrate ester bonds coupling long-chain fatty acids to alcohols. More preferably, the polypeptide of the invention hydrolyzes additionally ester bonds formed by short-chain fatty acids instead of long-chain fatty acids, which means that the preferred inventive lipase catalyzes both the hydrolysis of ester bonds formed by short- and long-chain fatty acids. Preferably, said long-chain fatty acids coupled to alcohols by ester bonds contain from 7 to at 30 carbon atoms, preferably from 8 to 28 carbon atoms, more preferably from 10 to 25 carbon atoms, even more preferably from 12 to 20 carbon atoms, most preferably from 15 to 18 carbon atoms. Preferably, the short-chain fatty acids contain from about 2 to about 6 carbon atoms. The fatty adds occurring in substrates of an inventive lipase may be aliphatic or non-aliphatic, saturated or non-saturated, substituted or non-substituted.

Furthermore, according to the invention it was established that an inventive lipase hydrolyzes specifically and exclusively sn-2 ester bonds of its substrates, in particular of triglycerides. As described above most of known lipases hydrolyze sn-1 or sn-3 ester bonds or sn-1 and sn-3 (1,3-positions) ester bonds of triglycerides. If any lipase known in the art hydrolyzes the sn-2 ester bond of triglycerides at all, this hydrolysis is accompanied by hydrolysis of sn-1 and/or sn-3 ester bond(s). Therefore, the present invention provides unexpectedly a preferably exclusively sn-2-specific lipase, which may provide new and advantageous lipid products. Consequently, a preferred embodiment relates to the polypeptide of the invention, which hydrolyzes preferably sn-2 ester bonds of its substrates, in particular of triglycerides. More preferably, the polypeptide of the invention hydrolyzes exclusively sn-2 ester bonds of its substrates, in particular of triglycerides. Typical substrates of inventive lipases are as disclosed above.

As mentioned above, the present invention also relates to functional fragments or derivatives of the polypeptide of the invention. The term “functional” is intended to define a polypeptide of the invention exhibiting lipase activity, particularly any lipase effect on lipase substrates. In particular, it relates to the hydrolysis of lipids and related molecules and beyond that to the catalytic activity on reactions associated with acyl groups. These include, e.g.:
hydrolysis: reaction of ester with water producing add and alcohol as well as with hydrogen peroxide to peroxy acids.
esterification: the reversal of hydrolysis; i.e. the production of ester from acid and alcohol.
alcoholysis: reaction of an ester with a monohydric alcohol such as ethanol, butanol, lauric alcohol or a polyhydric alcohol, such as glycerol, to produce an ester with a different alkyl group.
acidolysis: reaction of an ester with an acid leading to the exchange of acyl groups.
interesterification: reaction of one ester with another one leading to the randomization of acyl and alcohol moieties.

Thus, polypeptides of the invention encompassing lipase function are able to hydrolyze ester compounds as well as to catalyze the opposite reaction (synthesis of ester compounds). Accordingly, the inventive lipase may be used for the provision of various compounds, which are produced by the catalytic activity of an inventive lipase. These product compounds may exhibit more ester bonds than the corresponding educts (i.e. ester bond forming function) or may have less ester bonds than the educts (ester bond cleaving function, hydrolytic function of the inventive lipase).

Several methods for measuring enzymatic activity, including determination of lipase activity, are known by a person skilled in the art (e.g., enzyme assays using marked substrates, substrate analysis by chromatographic methods, such as HPLC or TLC for separating enzyme and substrate and spectrophotometric assays for measuring esterolytic activity) (see e.g., Maniatis et al. (2001) Molecular Cloning: A laboratory manual, Cold Spring Harbor Laboratory Press, Cold Spring Harbor, N.Y.).

The terms “fragment” and “derivative” of a polypeptide of the invention relate to the corresponding definitions given in the context of the esterases disclosed above, except that the functional fragments and derivatives of polypeptides of the present invention retain preferably their lipase activity (instead of esterolytic activity as mentioned above). The production and isolation of such polypeptide fragments or derivatives can be carried out as described above in the context of the esterases.

The polypeptide of the invention having lipase function can also be fused to at least one second moiety. Preferably, the second or further moiety/moieties does not occur in the natural lipase. The at least one second moiety can be an amino acid, oligopeptide or polypeptide and can be linked to the polypeptide of the invention at a suitable position, for example, the N-terminus, the C-terminus or internally. Linker sequences can be employed to fuse the polypeptide of the invention with at least one other moiety/moieties. According to one embodiment of the invention, the linker sequences preferably form a flexible sequence of 5 to 50 residues, more preferably 5 to 15 residues. In a preferred embodiment the linker sequence contains at least 20%, more preferably at least 40% and even more preferably at least 50% Gly residues. Appropriate linker sequences can be readily selected and prepared by a person skilled in the art. Additional moieties may be linked to the inventive sequence, if desired. If the polypeptide is produced as a fusion protein, the fusion partner (e.g., HA, HSV-Tag, His6) can be used to facilitate purification and/or isolation. If desired, the fusion partner can then be removed from the polypeptide of the invention (e.g., by proteolytic cleavage or other methods known in the art) at the end of the production process.

Another embodiment of the invention relates to a nucleic acid encoding a polypeptide of the invention having lipase function. Preferably, the nucleic acid comprises the nucleic acid sequence of FIG. 35. Also encompassed by the invention are nucleic acids encoding functional fragments or functional derivatives of the inventive polypeptide as described above.

Moreover, skilled artisans will recognize that the amino acids of polypeptides of the invention can be encoded by a multitude of different nucleic acid triplets because most of the amino acids are encoded by more than one nucleic acid triplet due to the degeneracy of the amino acid code. Since these alternative nucleic acid sequences would encode the same amino acid sequences, the present invention further comprises these alternate nucleic acid sequences coding for the same inventive amino acid sequence.

The nucleic acids of the invention can be DNA or RNA, for example, mRNA. The nucleic acid molecules can be double-stranded or single-stranded; single-stranded RNA or DNA can be either the coding (sense) strand or the non-coding (antisense) strand. If desired, the nucleotide sequence can include additional non-coding sequences such as non-coding 3′- and 5′-sequences or regulatory sequences. All nucleic acid sequences, unless otherwise designated, are written in the direction from the 5′ end to the 3′ end.

The nucleic acids of the invention can be fused to a nucleic acid comprising, for example, a marker sequence or a nucleotide sequence which encodes a polypeptide to assist, e.g., in isolation or purification of the polypeptide. Representative sequences include, but are not limited to those, which encode a glutathione-S-transferase (GST) fusion protein, a poly-histidine (e.g., His6), hemagglutinin, HSV-Tag.

Hybridization of nucleic acid strands can be used herein to analyze whether a given fragment or gene corresponds to the lipase described herein and thus falls within the scope of the pre-sent invention. Further details concerning the process of hybridization are described in the context of the first invention (directed to esterases) disclosed above and apply correspondingly.

Other embodiments of this second invention relate to a vector comprising an inventive nucleic acid coding for polypeptides having lipase function as well as a host cell comprising the vector and/or the nucleic acid of this second invention. All comments, terms (e.g. control sequences, other sequences etc.), explanations, definitions, examples (e.g. for expression vectors) etc. mentioned above concerning a vector, a host cell and the incorporation of the vector in said host cell in connection with the first invention (directed to esterases as first invention) disclosed above apply accordingly to a vector, a host cell and the incorporation of the vector in said host cell of the present invention.

Another embodiment of the invention provides a method for the production of the polypeptide of the invention having lipase function comprising the following steps:
(a) cultivating a host cell of the invention and expressing the nucleic acid under suitable conditions;
(b) isolating the polypeptide having lipase function by suitable means.

All comments, terms, explanations, definitions, examples for methods (e.g., activity-based screening method, in vitro transcription, solid phase peptide synthesis, suitable means for isolation etc.) mentioned above concerning the production and isolation of the polypeptide in connection with the esterases disclosed above apply accordingly to the production and isolation of inventive polypeptides having lipase function.

The polypeptides of the invention are usable in many applications, for example in olechemistry, in detergents, in paper manufacture, in the diary industry, as biocatalysts in organic syntheses, e.g., as acylating agents, in medicine, e.g. in the digestion of dietary fats, in substitution therapy, as anti-obesity agents, in food products and in the production or degradation of fats. This broad range of applications is due to the versatility of lipases, which catalyze various reactions, e.g. ester bond forming reactions and ester bond cleaving reaction in dependency upon the reaction parameters like temperature, substrate concentration etc. The products of lipase reaction catalyzed by the inventive enzyme may therefore comprise a large variety of molecules, e.g. (fatty) acids, in particular long-chain fatty acids (more than 6 carbon atoms) or alcohols resulting from the hydrolytic reaction. The fatty acids as products of the catalytic activity of the inventive lipase may be selected e.g. from any saturated or unsaturated (having one, two or more double bonds) fatty acids. In particular, saturated or unsaturated (with one or two double bonds) fatty acids with 12, 14, 16 or 18 or 20 carbon atoms are preferred. Any alcoholic compound, which is coupled by an ester bond to e.g. fatty acids may be prepared by the inventive lipase. Alcoholic compounds may be selected from any chemical compound showing an hydroxyl function (aromatic, aliphatic, heteroaromatic, heteroaliphatic, cyclic, heterocyclic compounds substituted i.a. by at least one hydroxyl group). A particular preferred product of a lipase-catalyzed reaction as provided by the present invention are triglycerides with the ester bond at the sn-2 position hydrolyzed. These products exhibit a hydroxyl group at sn-2, whereas at the sn-1 and sn-3 position are left ester bonded and are due to the sn-2 specificity of lipases of the invention. On the other hand, the product obtained from the ester bond forming function of an inventive lipase may be an ester, e.g. an ester from the class of fats, waxes, lecithins composed of alcoholic and (fatty) acid components.

Commercial oils and fats are produced at levels around 100 millions tons per annum and are used mainly for food (80%) and animal feed (6%) and for oleochemical purposes (14%). Thus, the polypeptide, nucleic acid, vector and/or cell of the present invention (hereinafter “substances of the invention”) are, for example, useful, for example, for the production of nutritional lipids and for the use in consumer products, particularly food products, preferably as food additive for the degradation of fat. For example, lipases can be used in cheese production. Addition of lipase(s) to a milk product (e.g., cow milk, goat milk, sheep milk) enhances the flavour of the cheese, accelerates the cheese ripening and/or assists in the preparation of “enzyme-modified cheeses” (EMC), an important commercial flavour used preferably in the USA for the manufacture of dips, sauces, dressings, crackers etc. EMC is produced from cheese curd by the addition of lipases at elevated temperatures, increasing the content of free fatty acids 10-fold.

Apart from their direct use in food, fats and oils are mainly used for oleochemical purposes. Therefore, another embodiment relates to the use of the substances of the invention (as an additive or component) in oleochemistry, in particular for the manufacture of oleochemicals, especially as catalyst for the manufacture of soap. The major oleochemical application of triglycerides is in the preparation of soaps. About two million tons of soap are produced annually by Monsovom, Sharples or De Laval—Centriput processes (Encyclopaedia of Chemical Technology, Eds. Kirk, R. E. and Othmer, D. F, Wiley, New York (1978)).

Another embodiment relates to the use of the substances of the invention for the preparation of a medicament in the treatment of digestive disorders and/or diseases of the pancreas. For example, lipases are useful in substitution therapy for the treatment of said disorders/diseases. Dietary fats are composed of about 95% triacylglycerols. Preduodenal and pancreatic lipases hydrolyzes these dietary triacylglycerols, wherein the pancreatic lipase hydrolyzes three, and preduodenal lipase hydrolyzes one of four acyl chains. In case a patient suffers from exocrine pancreatic insufficiency, it is possible to synthesize a recombinant lipase by genetic engineering techniques to compensate for the absence of the pancreatic lipase (so-called substitution therapy). These inventive substances, e.g. polypeptides with lipase function, may be administered orally or parentally in order to degrade excess fat substances prone to be enzymatically cleaved by the inventive lipase.

Furthermore, the substances of the invention are usable as enzymes (i.e. the inventive polypeptides exhibiting lipase activity) or for the production of enzymes (e.g. nucleic acids, vectors or cells producing these enzymes) for the treatment starting material in pulp and paper industry. For example, the addition of an inventive lipase to pulp and paper results in the hydrolysis of triglycerides (or other esters (e.g. fats)) contained therein. This results in a better “pitch control” for an easier processing of lumber to paper. A corresponding process is described by Fujita Y. et al. (Tappi J., 1992, 75, 117-122). The inventive substances may therefore serve as components or additives in the multi-step process for the preparation of e.g. paper.

Another embodiment relates to the use of the substances of the invention (for the preparation of a ((detergent) composition) or as an additive to (detergent) compositions) for the treatment of textiles or fabrics, in particular as ingredient in detergent compositions or fabric softener compositions. Standard detergent compositions contain anionic and non-ionic surfactants, oxidants and complexing agents at a pH of about 10. Lipases as detergent additives compete with chemical surfactants in the detergent composition, and thus enable the change of detergent formula in view of, e.g., lower wash temperatures and ecologically benign components. Moreover, lipases as detergent additive remove dyed soils, fat stains, wax esters, which are found in lipsticks, etc. from textiles. All comments, terms, explanations, definitions, examples etc. mentioned above in connection with the use of the esterases disclosed above in detergent compositions and in the treatment of cellulosic textiles or fabrics apply accordingly to the use of the substances of the invention in detergent compositions and in the treatment of textiles or fabrics.

As mentioned above, an example for the conversion of an esterase into a lipase is provided herewith. The short-chain specific and chemically stable esterase clone R.34 from rumen metagenome was converted into a lipase. Hereby, the amino acid sequence (FIG. 36) and nucleic acid sequence (FIG. 34) of R.34 are identical to the amino acid sequence (FIG. 27) and nucleic acid sequence (FIG. 15) of pBKR.34, as defined above, except that the sequences of pBKR.34 represent the full fragment whereas the sequences of R.34 represent only the gene sequence without the test of the fragment (flanking sequences).

Particularly preferred, the inventive polypeptide having lipase functionality is produced by directed evolution, in particular by conversion of an polypeptide having esterase activity (e.g. sequence 36) into a lipase polypeptide (e.g. sequence 37) of the invention. In this context, error-prone PCR mutagenesis and site-directed mutagenesis, e.g. as described in the following Examples 8 and 11 (and above in the context of the first invention), are the preferred methods to produce the polypeptide of the invention. However, any method suitable to produce the polypeptide and/or nucleic acid of the invention can be used.

One round of error-prone PCR mutagenesis was used to create a variant of the starting sequence with higher activity towards long-chain fatty acid esters. The improved variant lipase (EL1) was identified and sequence analysis of EL1 revealed a single amino acid substitution, N33D leading to a lipase exhibiting strong preference (>1000-fold) for the hydrolysis of esters bonds at the sn-2 position of long-chain triglycerides. Analysis of a three-dimensional model of EL1 revealed that the structural change underlying the properties of the inventive lipase enzyme consist in the formation of a salt bridge between the new D33 residue and R49. A consequence of the additionally introduced ionic part is a distortion of the enzyme structure that renders the catalytic site more accessible to larger substrates. The present invention shows that the substrate specificity of a true carboxyl-esterase (R.34) can be engineered towards insoluble substrates, i.e. converted into a true lipase (e.g. EL1), without modifications on the shape, size and hydrophobicity of the substrate-binding sites that are being considered the key to influence the esterase/lipase chain-length specificity (Klein, R. R et al., Lipids 1997, 32, 123-130; Eggert, T. et al., Eur J. Biochem. 2000, 267, 6459-6469; Kauffmann, L. et al., Protein Eng. 2001, 14, 919-928; Yang, J. et al., Protein Eng. 2002, 15, 147-152)

In summary, the present invention provides a novel stereoselective lipase acting preferably on the sn-2 position of triglycerides and was engineered from a short-chain specific esterase isolated from rumen fluid metagenome. The remarkable feature of this finding is that—in contrast to the action of other lipases—the polypeptides according to the invention are the only enzymes capable of preferably or exclusively hydrolyzing secondary (and not primary) ester bonds from triglycerides known so far. This represents an important step, e.g., towards the synthesis of nutritional lipids not being obtainable by prior art enzymes.

The following Figures and Examples are intended to illustrate the invention without limiting the scope of the invention. All references cited herein are incorporated in their entirety.

### FIGURES

FIG. 34 shows the nucleic acid sequence of wild type R.34,

FIG. 35 shows the nucleic acid sequence of EL1 sequence having lipase functionality,

FIG. 36 shows the amino acid sequence of wild type R.34,

FIG. 37 shows the amino acid sequence of EL1, an inventive polypeptide having lipase activity,

FIG. 38 shows esterase phenotype of E. coli TOP10 cells expressing EL1 improved variant (left side) and wild type esterase R.34 (right side) (both cloned in PCR2.1 cloning vector). Cells were plated onto fresh LB-plate containing kanamycin (50 μg/ml). Plates were incubated for 12 h at 37° C., and then the plates were covered with a second layer containing the substrate (20 ml Tris-HCl 50 mM, pH, 0.4% agarose, 320 μl of Fast Blue RR solution in DMSO [80 mg/ml] and 320 μl of αNL solution in acetone [20 mg/ml]). Positive clones are visible due to the formation of a brown precipitate.

Firstly, one round of error-prone PCR mutagenesis was used to create a mutant with higher activity towards long-chain fatty acid esters. The starting material of this mutagenesis was R.34 (for further details see Example 8). Potential improved variants were identified on agar plates by using α-naphthyl laurate (αNL) and azo dye (Fast Blue RR) that reacts with the released 2-naphthol to generate an insoluble brown product (Khalameyzer, V. et al., Appl Environ Microbiol. 1999, 65, 477-482). As can be seen, E. coli colonies expressing wild type R.34 produced no brown zones on αNL-plates (FIG. 38 A) but produced them on αNA-plates (α-naphthyl acetate) (FIG. 38 B). Approximately 8,200 colonies were screened in the first round and only one clone (EL1) was identified. Such a low frequency of improvement was surprising and estimated the fact that long-chain esters are very inefficient substrates for R.34. The EL mutant identified by the plate assay (see FIGS. 38 A and B) was isolated and a His tag was added to the C-terminus to allow its easy purification.

FIG. 39 shows SDS-PAGE of the purified wild type R.34 and EL1 mutant in order to compare R.34 and EL1 in more detail (for further details see Example 10). About 2.2 mg of pure recombinant protein per g wet weight cells were recovered by a one-step purification method involving metal-chelating chromatography. Samples were loaded as follows: MW, molecular mass markets (15-100 kDa, Novagen); lane 1, crude extract of E. coli TOP10 harbouring PCRR.34 after induction with IPTG; lane 2, crude extract of E. coli TOP10 harbouring PCREL1 after induction with IPTG; lane 3, purified R.34 with His₆ tag at the C-terminus; lane 4, purified EL1 with His₆ tag at the C-terminus. Both proteins possess a molecular weight of about M, 25 kDa.

FIG. 40 A-D shows the relative activity of wild-type esterase R.34 and EL1 mutant to different substrates with varying chain lengths. FIG. 40 A shows the relative activity of wild-type R.34 to p-nitrophenyl esters [p-NP esters], FIG. 40 B shows the relative activity of wild-type R.34 to triacylglycerols. FIG. 40C shows the relative activity of EL1 mutant to p-nitrophenyl esters [p-NP esters], FIG. 40 D shows the relative activity of EL1 mutant to triacylglycerols. Specific activities are given in units/mg pure protein.

As shown in FIG. 40 A and B R.34 preferentially hydrolyses triacylglycerol and p-NP esters of fatty acid with short-chain length of C≦4, being optimal for C₃. These results provide evidence that R.34 is a true esterase. In contrast, EL1 preferentially hydrolyses substrates with long-chain length. The optimal acyl chain specificity for p-NP esters switches from C₃ to C₁₂ (p-NP laurate) with nearly one order of magnitude increase in specific activity (FIG. 40 C). Moreover, the specificity switches off >1,000-fold towards short-chain triacylglycerols. Although, tributyrin (C₄) was the optimal substrate for EL1 (214,000 units/mg), it was also able to hydrolyse efficiently typical lipase substrates, such as trilaurin, tripalmitin and triolein (over 67,000 units/mg) (FIG. 40 D). R.34 was not able to hydrolyze triacylglycerols with chain lengths of more than C₄ (the result for C₆ is negligible).

FIG. 41 represents circular dichroism studies. FIG. 41A shows Fat-UV CD spectra of wild type R.34 and EL1. FIG. 41B shows unfolding profiles of wild type R.34 and EL1. The samples were heated at 1° C./min. from 15 to 90° C. and the ellipticity was recorded at 222 nm. The CD spectra were measured at 25° C. The Tₘ values were calculated by a non-linear least-squares fit of the transition temperatures.

The CD spectra for wild type R.34 and EL1 were almost similar and showed minima at 208 and 222 nm (FIG. 41 A). This profile is consistent with a α-helical protein. The thermal unfolding of each protein was determined by fitting the ellipticity at 222 nm (θ₂₂₂) versus temperature (FIG. 41 B). As shown in FIG. 41 B the Tₘ for EL1 shifted from 63.7° C. (for wild type R.34) to 51.3° C. (for EL1).

FIG. 42 represents parameters affecting activity of wild type R.34 and EL1. The measurement was carried out spectrophotometrically following an increase in the absorbance at 410 nm due to hydrolysis of p-NP propionate. The relative activity of the enzymes to p-NP propionate is normalized. FIG. 42 (A) shows the results of inactivation experiments by addition of active site inhibitors. After incubation (1 mM) of PMSF (seine esterase inhibitor, control), capryl sulphonyl fluoride (C6SF), lauryl sulfonyl fluoride (C12SF) and palmityl sulphonyl chloride (C16 SF) the hydrolytic activity was monitored. While the activity of R.34 is maintained or only slightly reduced, EL1 shows strong reduction of its enzymatic activity for all compounds tested (short, medium and long fatty acid sulfonyl fluorides), which documents that the serine residue at the active site is more accessible in EL-1 than in the wild-type. Further, it was tested whether the improved lipase variant was more susceptible to detergents and solvents. The effect of ionic Triton X-100 (C) on esterase and mutated esterase (lipase) activity was measured. The EL-1 variant was more affected than the wild type enzyme. Whereas R.34 showed maximum activity at 0.6% (w/v) Triton X-100 and retained more than 50% of the maximum activity at 5% (w/v), the EL-1 variant was strongly inhibited above 0.6% (w/v). This shows that the mutation of EL-1 induces a conformational change to facilitate catalytic residues to be exposed to big detergent micelles. As illustrated in FIG. 42, stability of the mutant was highly similar that that of the wild type upon addition of acetonitrile (B). This suggests that solvent-exposed residues are not likely to change upon mutations and that they are equally exposed in both variants.

FIG. 43 shows a TLC analysis of products of triolein hydrolyzed by EL1 mutant, Rhizomocur miebei lipase (1,3-specific) (Novozymes), Candida antarctica A (no specific) (Novozymes). Control: triolein substrate without enzyme. The procedures used for enzyme and substrate preparation, hydrolysis, TLC, and visualization are described in Example 10. Abbreviation: TG—triglyceride; DG—diglyceride; MG—monoglyceride.

The aim was to explore the biotechnological potential of the new created lipase EL1 compared to other lipases. Therefore, the positional specificity of EL1 mutant using triolein was examined by thin-layer chromatography. The reactions were carried out until the extent of hydrolysis was 25%, which was reached in 1-2 min. Spontaneous acyl migration was considered negligible because of the short reaction time. As shown in FIG. 43, EL1 is highly specific to the 2-position (sn-2 specific), in particular at lower incubation time. This contrasts with Candida antarctica A lipase, which showed any ester bond preference and hydrolyzed ester bonds at the sn-1 or sn-3 as well as at sn-2 and Rhizomocur miebei lipase, which has preference for the hydrolysis of ester bonds at the sn-1 and sn-3. Using these conditions wild type R.34 did not hydrolyze triacylglycerols>C₄.

FIG. 44 shows sequence alignments of wild type esterase R.34 and other xylanases and esterases. Source organisms and accession numbers are as follows: R.34 (described herein); B.fib=beta-1,4-D-xylanase from Butyrivibrio fibrisolvens (accession number X61495.1); C.ace=acetyl esterase family enzyme from Clostridium acetobutylicum ATCC 824 (accession number NC_{—}003030.1); EST2=esterase from A. acidocaldarius (PDB Acc. number 1EVQA; crystal structure resolved). Sequence inspection allowed the identification of residues Ser₁₃₇, Asp₂₁₅ and His₂₄₇ as the catalytic residues of R.34 esterase (shown with asterisk). Mutated residue is shown by an arrow (▴). As can be observed, sequence similarity extends along all the protein sequence with the exception of the 1-25 region (R.34 numbering). Secondary structure data allowed to divide R.34 in regions where sequence similarity alone provides unambiguous results.

FIG. 45 represents three-dimensional structures. FIG. 45 A represents an overall three-dimensional structure of R.34 obtained by homology modelling. Residues belonging to the catalytic triad and N33 are explicitly shown. FIG. 45 B shows a schematic representation of the putative salt bridge binding residues D33 and R44 in the EL1 mutant.

The sequence analysis of EL1 revealed a single amino acid substitution, namely N33D. The question rising from this finding was, why this single ammo acid substitution in EL1 mutant has such a profound effect on the substrate specificity. In order to explain the significant differences observed in specific activity mediated by this substitution, a three-dimensional model of R.34 structure was produced. For that, the esterase sequence was aligned with that of A. acidocaldarius (for further details see Example 13). As can be seen from FIG. 45 A the substitution of Asn33 (N33) by Asp (D) leads to the formation of an ionic pair between the newly introduced Asp33 (D33) and Arg49 (R49) (see FIG. 45 B). Most likely, this causes a distortion of the enzyme structure that makes the catalytic site more accessible to larger substrates.

FIG. 46 shows esterase phenotype of E. coli TOP10 cells expressing R.34 variants (pCR.2.1 cloning vector).

To confirm the supposed interaction between D33 and R49 (as described in FIG. 45) and that this interaction affects the catalytic activity of the mutant enzyme towards triacylglycerols, single R49D and R49N mutant variants of the enzyme were generated by site-directed mutagenesis. Mutated proteins were cloned into pCR2.1 plasmid (Invitrogen) and expressed in E. coli TOP10. Cells were plated onto fresh LB-plate containing 50 μg/ml Kanamycin. Plates were incubated for 12 h at 37° C. together with E. coli TOP10 cells expressing wild type R.34 and EL1 mutant and then covered with a second layer containing the substrate (20 ml Tris-HCl 50 mM, pH, 0.4% agarose, 320 μl of Fast Blue RR solution in DMSO [80 mg/ml] and 320 μl of αNL solution in acetone [20 mg/ml]).

Positive clones are visible due to the formation of a brown precipitate. As can be seen, E. coli colonies expressing wild type R.34, EL1_{R49D} and EL1_{R49N} produced no clear zones on αNL-plates (FIG. 46 A), but they produced on αNA-plates (FIG. 46 B). Therefore, all variants, except that containing the N33D mutation (EL1), were unable to hydrolyze αNL. This revealed that both D33 and R49 residues, participate in the acyl chain length preference of R.34 enzyme. Mutations at R49 produced variants with lower or no hydrolytic activity towards long-chain fatty acyl substrates.

### Examples

### Materials

### Reagents, Strains and Buffers:

p-nitrophenyl esters (p-NP esters), triacylglycerols, Fast Blue RR, α-naphthyl acetate (αNA) and laurate (αNL), and phenyl methyl sulphonyl fluoride (PMSF) were purchased from Sigma Chemical Co. (St. Louis, Mo., USA). Caproyl-, lauryl- and palmitoylsulphonyl fluoride were synthesized as described by Deutsch, D. G., et al. Biochem. Biophys. Res. Commun. 1997, 231, 217-221. Unless noted otherwise, esters for the chiral substrate library were purchased from Aldrich or Fluka (Oakville, Canada). All other chemicals were of analytical grade. Molecular mass markets for SDS-PAGE were obtained from Novagen (Madison, Wis., USA). Restriction and modifying enzymes were from New England Biolabs (Beverly, Mass., USA). DNase I grade II was from Boehringer Mannheim (Mannheim, Germany). Chromatographic media and molecular markets for native electrophoresis, were from Amersham Pharmacia Biotech (Little Chalfont, UK). E. coli strains XL1-Blue MRF′ (for library construction and screening), XLOLR (for expression of the esterase from phagemid) (both—Stratagene; La Jolla, Calif., USA), and TOP 10 (for site-directed mutagenesis and expression of mutant esterases) (Invitrogen; Carlsbad, Calif., USA), were maintained and cultivated according to the recommendations of suppliers and standard protocols described elsewhere (Sambrook, J., ritsch, F., Maniatis, T, 1989. In: Molecular Cloning A Laboratory Manual (Cold Spring Harbor: Cold Spring Harbor Laboratory Press, 1989). [2nd ed.]). Unlike otherwise indicated, the standard buffer used in the present study was 100 mM Tris-HCl buffer, pH 8.5.

### Source of Enzyme:

DNA manipulations were according to Sambrook et al. (1998) and according to manufacturer's instructions for the enzymes and materials employed. Wild type esterase was retrieved from the bacteriophage lambda-based expression library created from DNA extracted from cow rumen fluid, after the screening in NZY soft agar containing αNA, and expressed from the pBK-CMV phagemid pBKR.34 in E. coli XLOLR as will be described in elsewhere (Ferrer et al., unpublished). The sequences of pBKR.34 (FIGS. 15 and 27) represent the full fragment. The sequences of R.34 (FIGS. 34 and 36) represent the gene sequence without flanking sequences.

### Example 8

### PCR Mutagenesis

Error-prone PCR mutagenesis was carried out using Genemorph kit from Stratagene, according to the manufacturer instructions, except that 3% (v/v) dimethyl sulfoxide (DMSO) was included in the reaction mixture. Phagemid pBKR.34 was used as template for mutagenesis. The amplification programme was as follows: 2 min at 95° C., 27 sec at 94° C., 27 sec at 53° C., followed by 28 cycles of 3 min at 74° C., and 10 min at 74° C.

Primers sequences were as follows:

OligF sense (5′-CCT ATC CCT ATA CCA TTG C-3′) and OligR antisense (5′-CCG TCC ATA TAA TAC TTC AGG-3′). The amplified PCR products were purified from a 0.75% agarose gel using QIAEX II gel extraction kit from QIAGEN, cloned into plasmid pCR2.1 (Invitrogen) and transformed into E. coli TOP10 (Invitrogen) as recommended by the supplier, and the resulting transformants plated onto fresh LB-plate containing and 50 μg·mL−1 kanamycin. Plates were incubated for 12 h at 37° C. and then the plates were covered with a second layer containing the substrate (20 ml Tris-HCl 50 mM, pH 8.0, 0.4% agarose, 320 μl of Fast Blue RR solution in DMSO [80 mg/ml] and 320 μl of αNL solution in acetone [20 mg/ml]). Positive clones appeared due to the formation of a brown precipitate. Using these conditions E. coli colonies expressing wild type R.34 produced no clear zones on αNL plates. Positive transformant of E. coli was pooled and the plasmid DNA was isolated using a QIAprep spin miniprep kit (QIAGEN).

### Example 9

### Expression and Purification of Enzyme Variants

To determine the biochemical properties of the R.34 and EL1 variants, the gene corresponding to the full-length protein was amplified and produced as fusion with a hexahistidine His₆ tag at the C-terminus R.34_{His}, or EL1_{His}) as follows. The esterase-encoding gene was amplified from pBKR.34 or pCREL1 plasmids, by PCR with oligonucleotide primers designated Mut34FpCR sense: 5′-CCT ATC CCT ATA CCA TTG CTT-3′ and Mut34RpCR antisense: 5′-TTT AGT GGT GGT GGT GGT GGT GCT TGA TCC TGA TCT TTT TCC CTT CGG T-3′. Reactions were carried out in a total volume of 50 μL and were catalyzed by 25 U of Taq polymerase (Qiagen). The amplification program was as follows: 1 min 94° C. followed by 25 cycles of 20 sec. 94° C., 60 sec. 40° C., 1 min 72° C., the final elongation step was 5 min 72° C. and 15 min 10° C. The amplified fragments, purified from a 0.75% agarose gel, were cloned into plasmid pCR2.1 (Invitrogen) and electroporated into E. coli TOP10 (Invitrogen) as recommended by the supplier. E. coli TOP10 transformed with the expression plasmids (pCRR.34_{His} and pCREL1_{His}) were grown overnight at 37° C. in Luria-Bertani medium supplemented with 50 μg·mL⁻¹ kanamycin. Isopropyl thio-β-D-galactoside (IPTG) was added to a concentration of 1 mM and cultivation was continued for an additional 4 h. Cells were collected by centrifugation (30 min, 8000 g, 4° C.) and resuspended in 20 mM NaH₂PO₄ pH 7.4, 150 mM NaCl and 20 mM imidazole. After the addition of lysozyme (1 mg·mL⁻¹), the suspension was incubated on ice for 30 min and then sonicated four times for 30 s. The cell lysate was centrifuged for 20 min at 4° C., 25000 g. The His₆-tagged enzyme was purified at 4° C. on HisTrap HP column (Amersham Pharmacia Biotech; Little Chalfont, UK). After washing with 4 ml of 20 mM NaH₂PO₄ pH 7.4, 150 mM NaCl and 20 mM imidazole, the recombinant enzyme was eluted at pH 7.4 with 10 ml of 20 mM NaH₂PO₄, 150 mM NaCl and 500 mM imidazole. Purification of the recombinant proteins was monitored spectrophotometrically following the increase in absorbance at 405 nm due to hydrolysis of p-NP propionate.

### Example 10

### Protein Characterization

SDS PAGE was performed using 12% (v/v) acrylamide gels according to Laemmli (Laemmli, U. K. Nature 1970, 227, 680-685. The protein concentration was determined according to Bradford (Bradford, M. M. Anal. Biochem. 1976, 72, 248-254) with BSA as the standard.

Hydrolytic activity was determined spectrophotometrically at 40° C. using p-NP esters ranging from acetate to palmitate as substrates, as described by Ferrer et al. (Ferrer, M. et al., Chem. Biol. 2005, In Press), although with small modifications. Briefly, the reaction mixture (3 ml of 50 mM Tris-HCl pH 8.0) contained 0.2 mM p-NP esters with 0.2% (w/v) arabic gum. p-NP propionate was the esterase substrate for activity determination, if not otherwise stated. Lipase activity was determined at 40° C. in a pH-stat assay (San Clemente and Vadegra, 1967) by titrating fatty acids released from triacylglycerols ranging from triacetin to triolein, with 0.1 M sodium hydroxide in a pH-stat Mettler Toledo (model DL50-Graphix) (Metrohm). The reaction mixture (20 ml of 1 mM Tris-HCl, pH 8.0) contained emulsions of 80 mM triacylglycerols (C₂-C₄) or 40 mM (C₆-C_{18:1}) with 0.2% (w/v) arabic gum. Tripropionin was the lipase substrate for activity determination, if not otherwise stated. Esterase chiral-substrate ranges and calculation of apparent enantiomeric ratios for each substrate and enzyme pair were performed at 40° C. as described previously (Ferrer, M. et al., Chem. Biol. 2005, In Press; Janes, L. E. et al., Chem. Eur. J. 1998, 4, 2317-2324) in 96-well microtiter plates containing 5 μg of pure protein, 10 mM substrate, 0.8 mM resorufin acetate as internal standard and 0.911 mM phenol red, in 200 μl of 5 mM EPPS buffer (N-(2-hydroxyethyl) piperazine-N′-(3-propanesulfonic acid; pH 8.0) per well, and monitored colorimetrically at 550 nm. In all cases, one unit of enzyme is defined as the amount of enzyme liberating 1 μmol product per min, under experimental conditions. All values were determined in triplicate and were corrected considering the autohydrolysis of the substrate. Positional specificity of the lipase EL1 was examined by thin-layer chromatography (TLC) of the reaction product obtained by using pure triolein (Sigma Chemicals Co.) as a substrate. A reaction mixture composed of 5 ml of 100 mM Tris-HCl buffer (pH 8.5), containing 0.2% arabic gum (Sigma Chemicals Co.), 0.5 g of triolein and 50 μg of the enzyme, were incubated at 30° C., with orbital shaking at 1000 rpm. After incubation, the reaction products from 5 ml-individual reactions incubated for a period of time ranging from 0 to 45 min, were extracted with 25 ml of ethyl ether and an aliquot was applied to a Silica gel and developed with a 97:2:1 (v/v) mixture of chloroform, acetone, and acetic acid. The spots were visualized by spraying the plate with 95% (v/v) H₂SO₄ in ethanol and then heating in an oven at 150° C. until charring occurred.

The pH and temperature optima were investigated in the range of 5-12 and 25-70° C., respectively. The buffers (100 mM) used were: citrate (pH 5.0-5.5), MES (pH 5.5-7.0), HEPES (pH 7.0-8.0), Tris-HCl (pH 8.0-9.0) and glycine-NaOH (pH 9.0-12). The influence of cations and solvents on enzyme activity was analyzed by adding the chloride salts and solvents to the standard esterase solution to final concentrations of 400 mM and 0-70% (v/v), respectively. Activity measurements were made immediately and after 720 min of incubation, at 40° C. Detergents were tested at 50 mM (for sodium dodecyl sulfate, SDS) or from 0 to 7% (w/v) for Triton X-100. Residual activity was expressed as percent of the control value obtained without addition of chemical. All values were determined in triplicate and were corrected considering the spontaneous hydrolysis of the substrate.

### Example 11

### Site-Directed Mutagenesis

EL1 esterase mutants were prepared using a QuikChange XL site-directed mutagenesis kit (Stratagene), according to the vendor's instructions. The oligonucleotides used for mutagenesis were as follows. R49D: 5′-GCC TCA AGA TAT TCg acG CAC CTG ATG ACA AGG-3′ and 5′-CCT TGT CAT CAG GTG Cgt cGA ATA TCT TGA GGC-3′; R49N: 5′-GCC TCA AGA TAT TCA acG CAC CTG ATG ACA AGG-3′ and 5′-CCT TGT CAT CAG GTG Cgt TGA ATA TCT TGA GGC-3′. E11-derived plasmids containing mutations were introduced into E. coli TOP10 by electroporation.

### Example 12

### DNA Sequencing

Plasmids containing mutant genes were sequenced at the Sequencing Core Facility of the Instituto de Investigaciones Biomédicas (CSIC, Madrid) using an Applied Biosystems 377 automated fluorescent DNA sequencer. The primers used were as follows. F1: 5′-AAC AAC AAG GCC TTC CTG CGC-3′, F2: 5′-TGG GCG TGC TTA CCT ACA CCG-3′, and F3: 5′-ACA TCT GCT GGG CAG ACA ACG-3′.

### Example 13

### Molecular Modeling

Multiple sequence alignments of protein homologues to R.34 esterase were generated by GenTHREADER (Jones, D. T. J. Mol. Biol. 1999, 287, 797-815) using the following hydrolase sequences: beta-1,4-D-xylanase from Butyrivibrio fibrisolvens (accession number X61495.1), acetyl esterase family enzyme from Clostridium acetobutylicum ATCC 824 (accession number NC_{—}003030.1), EST2-esterase from A. acidocaldarius (De Simone, G. et al., J. Mol. Biol. 2000, 303, 761-771) (PDB accession code 1EVQA) and R.34 (R.34, this work). The alignment featuring the highest score was obtained using the Blosum matrix (Henikoff, S. et al., Proc. Natl. Acad. Sci. USA 1992, 89, 10915-10919) and standard CLUSTALX parameters. The structure of esterase EST2 from A. acidocaldarius was chosen as the most suitable template to generate a model for R.34. Model coordinates were obtained from the Swiss-Model server (Guex, N. et al., Electrophoresis 1997, 18, 2714-2723; Guex, N. et al., Trends Biochem. Sci. 1999, 24, 364-367) and analysed with Swiss-PDB Viewer program (Guex, N. et al., Electrophoresis 1997, 18, 2714-2723).

### Supporting Tables

TABLE S1
Properties, subunit composition and putative catalytic triad
of the recombinant R.34 enzymes from rumen metagenome

| | |
|---|---|
| Temperature optimum (° C.) | 50 |
| Optimum pH | 7.5 |
| Number of amino acids | 273 |
| pH stability^{a} | 9.0 |
| T stability (° C.)^{a} | 56 |
| Apparent Mr (kDa) | |
| Native enzyme | 26.00 |
| Subunit^{b} | 25.81 |
| Subunits | 1 |
| pI^{b} | 4.57 |
| Putative catalytic Ser | S₁₃₇ [GDS(L)] |

^{a}The esterase activity:pH and activity:temperature relationships were determined by incubating the standard enzyme:substrate mixtures at different pH values and constant temperature (40° C.), and at different temperatures (15-80° C.) and constant pH (8.5), respectively. Aliquots (100 μl) were taken at intervals and the remaining activity was measured using the standard assay, after adding the substrate. All values were determined in triplicate and were corrected considering the autohydrolysis of the substrate. pH and thermal stability refer to the values at which the activity is 80% of the optimum being t_{1/2} >30 min.
^{b}Theoretical molecular masses and isoelectric points determined by translation
of gene sequence.

TABLE S2
Specific activities of purified rumen esterase R.34.

| | Specific activity |
|---|---|
| Substrate | (units/mg) |
| p-Nitrophenyl esters | |
| p-nitrophenyl Acetate | 63.0 |
| p-nitrophenyl Propionate | 449.0 |
| p-nitrophenyl Butyrate | 142.6 |
| p-nitrophenyl Caproate | 93.2 |
| p-nitrophenyl Caprylate | 20.3 |
| p-nitrophenyl Laurate | — |
| p-nitrophenyl Myristate | — |
| p-nitrophenyl Palmitate | — |
| Triacylglycerols | |
| Triacetin | 162.2 |
| Tripropionin | 350.5 |
| Tributyrin | 130.4 |
| From tricaproin to triolein | — |

^{a}Reaction conditions: [E] = 5 μg, [substrate] = 0.2 mM for p-NP-esters or 150 mM for triglycerides, 100 Tris-HCl, pH 8.5, T = 40° C. For hydrolysis of p-NP esters, 150 μl of a 16 mM p-NP-ester stock solution in acetone (Sigma) were incubated for 2 min with 5 μg enzyme diluted in 2850 μl of 100 mM buffer containing 0.2% (w/v) arabic gum, and followed spectrophotometrically at 405 nm. Tritration of free fatty acid released by hydrolysis of triglycerols was followed in a pH-stat (Mettler, model DL50), using 0.1 M NaOH as titrant. All values were determined in triplicate and were corrected considering the spontaneous hydrolysis of the substrate. Results shown are the average of three independent assays.
—^{b} no hydrolysis product detected.

TABLE S3
Specific activities of rumen R.34 esterase towards enantiomers

| | Specific activity |
|---|---|
| | (units/mg)^{a} |
| Solketal acetate | 573 |
| Neomenthyl acetate | —^{b} |
| Menthyl acetate | — |
| Methyl 3-hydroxybutyrate | 349 |
| Pantolactone | — |
| Methyl-3-bromo-2-methylpropionate | 507 |
| Methyl-3-hydroxy-2-methylpropionate | 310 |
| Dihydro-5-hydroxymethyl-2(3H)-furanone | — |
| Alanine methyl ester | — |
| Tryptophan methyl ester | — |
| Methyl lactate | — |
| N-benzyl-proline-ethyl ester | — |

^{a}All measurements were performed three times under the following conditions: 96-well microtiter plates containing 5 μg of pure protein, 10 mM substrate, 0.8 mM resorufin acetate as internal standard and 0.911 mM phenol red, in 200 μl of 5 mM EPPS buffer (N-(2-hydroxyethyl)piperazine-N′-(3-propanesulfonic acid; pH 8.0) per well, and monitored colorimetrically at 550 nm. Hydrolytic activities were measured at 40° C. Activity values are given as the average of the hydrolytic rates for both pure enantiomers (R/S or D/L), measured at 40° C.
—^{b} no hydrolysis product detected.

TABLE S4
Enantiomeric ratio, Eₐₚₚ [Stereo-preference]^{a}

| | Enantiomeric ratio, Eₐₚₚ | |
|---|---|---|
| | [Stereo-preference]^{a} | |
| Solketal acetate | 18.5 | [R] |
| Neomenthyl acetate | —^{b} | |
| Menthyl acetate | — | |
| Methyl 3-hydroxybutyrate | 117 | [S] |
| Pantolactone | — | |
| Methyl-3-bromo-2-methylpropionate | 54.1 | [S] |
| Methyl-3-hydroxy-2-methylpropionate | 2.4 | [S] |

| | |
|---|---|
| Dihydro-5-hydroxymethyl-2(3H)-furanone | — |
| Alanine methyl ester | — |
| Tryptophan methyl ester | — |
| Methyl lactate | — |
| N-benzyl-proline-ethyl ester | — |

^{a}All measurements were performed three times under the following conditions: 96-well microtiter plates containing 5 μg of pure protein, 10 mM substrate, 0.8 mM resorufin acetate as internal standard and 0.911 mM phenol red, in 200 μl of 5 mM EPPS buffer (N-(2-hydroxyethyl)piperazine-N′-(3-propanesulfonic acid; pH 8.0) per well, and monitored colorimetrically at 550 nm. Apparent enantiomeric ratios for each substrate and enzyme pair were performed as described by Janes et al. (1998).
—^{b} no hydrolysis product detected.

1. Polypeptide comprising an amino acid sequence of amino acids No. 90 to No. 120 of one of the amino acid sequences shown in FIGS. 22 to 33 or a functional fragment, or functional derivative thereof.

2. Polypeptide of claim 1, wherein the polypeptide comprises an amino acid sequence of amino acids No. 90 to No. 120, preferably No. 85 to No. 135, more preferably No. 70 to No. 160 most preferably No. 60 to No. 175 of one of the amino acid sequences shown in FIGS. 22 to 33.

3. Polypeptide of claim 1, wherein the polypeptide comprises one of the amino acid sequences shown in FIGS. 22 to 33.

4. Polypeptide of claim 1, wherein the polypeptide hydrolyzes p-nitrophenyl acetates and/or p-nitrophenyl esters, preferably p-nitrophenyl esters containing from 2 to 12 carbon atoms.

5. Polypeptide of claim 1, wherein the polypeptide releases covalently bound lignin from hemicelluloses.

6. Polypeptide of claim 5, wherein the polypeptide represents a feruloyl esterase.

7. Polypeptide of claim 1, wherein the polypeptide releases acetic acid from carbohydrates.

8. Polypeptide of claim 7, wherein the polypeptide represents a carbohydrate esterase.

9. Polypeptide of claim 1, wherein the polypeptide is derived from rumen, particularly from rumen ecosystem, preferably from cow rumen, more preferably from New Zealand dairy cow.

10. Polypeptide of claim 1, wherein the polypeptide shows activity at pH optimum, preferably at pH ranging from 7.5 to 12.0, more preferably from 7.5 to 8.5 or from 9.5 to 10.0 or from 11.0 to 12.0.

11. Polypeptide of claim 1, wherein the polypeptide shows activity at temperature optimum, preferably at a temperature from 40° C. to 60° C., more preferably 40° C. to 50° C. or from 50° C. to 60° C.

12. Polypeptide of claim 1, wherein the polypeptide shows activity at low addition of cations, preferably without any addition of cations.

13. Polypeptide of claim 1, wherein the polypeptide shows high specific activity towards its substrate.

14. Polypeptide of claim 1, wherein the polypeptide shows high stability towards its substrate.

15. Polypeptide of claim 1, wherein the polypeptide shows high enantio-selectivity towards its substrate.

16. Polypeptide of claim 1, wherein the polypeptide shows a combination of at least two features, preferably three features, more preferably four features, even more preferably five features, most preferably six features selected from:
a. hydrolyzing p-nitrophenyl esters containing from 2 to 12 carbon atoms;
b. releasing covalently bound lignin from hemicelluloses;
c. representing a feruloyl esterase;
d. releasing acetic acid from carbohydrates;
e. representing a carbohydrate esterase;
f. being derived from rumen, particularly from rumen ecosystem preferably from cow rumen, more preferably from New Zealand dairy cow;
g. showing activity at pH optimum, preferably at pH ranging from 7.5 to 12.0, more preferably from 7.5 to 8.5 or from 9.5 to 10.0 or from 11.0 to 12.0;
h. showing activity at pH optimum, preferably at pH ranging from 7.5 to 12.0, more preferably from 7.5 to 8.5 or from 9.5 to 10.0 or from 11.0 to 12.0;
i. showing activity at low addition of cations, preferably without any addition of cations;
i. showing high specific activity towards its substrate;
k. showing high stability towards its substrate; and
l. showing high enantio-selectivity towards its substrate.

17. A nucleic acid encoding a polypeptide of claim 1 or a functional fragment or functional derivative thereof.

18. The nucleic acid of claim 17 comprising or consisting of one of the nucleic acid sequences of FIGS. 10 to 21.

19. A vector comprising the nucleic acid of claim 17.

20. A host cell comprising the vector of claim 19.

21. A method for the production of the polypeptide of claim 1 comprising the following steps:
a. cultivating a host cell, said host cell comprising a nucleic acid encoding said polypeptide, and expressing the nucleic acid under suitable conditions; and
b. isolating the polypeptide with suitable means.

22. (canceled)

23. (canceled)

24. (canceled)

25. Polypeptide comprising the amino acid sequence of amino acids No. 20 to No. 50 of the amino acid sequence shown in FIG. 37 or a functional fragment, or functional derivative thereof.

26. Polypeptide of claim 25, wherein the polypeptide comprises the amino acid sequence of amino acids No. 18 to No. 70, more preferably No. 15 to No. 100, even more preferably No. 10 to No. 130 of the amino acid sequence shown in FIG. 37 or a functional fragment, or functional derivative thereof.

27. Polypeptide of claim 25, wherein the polypeptide comprises the amino acid sequence shown in FIG. 37.

28. Polypeptide of claim 25, wherein the polypeptide hydrolyzes ester bonds of saturated or unsaturated, substituted or unsubstituted long-chain fatty acids.

29. Polypeptide of claim 28, wherein the long-chain fatty acids contain from 7 to 30 carbon atoms, preferably from 8 to 28 carbon atoms, more preferably from 10 to 25 carbon atoms, even more preferably from 12 to 20 carbon atoms, most preferably from 15 to 18 carbon atoms.

30. Polypeptide of claim 25, wherein the polypeptide hydrolyzes preferably sn-2 ester bonds of its substrates.

31. A nucleic acid encoding a polypeptide of claim 25 or a functional fragment or functional derivative thereof.

32. The nucleic acid of claim 31 comprising the nucleic acid sequence of FIG. 35.

33. A vector comprising the nucleic acid of claim 31.

34. A host cell comprising the vector of claim 33.

35. A method for the production of the polypeptide of claim 25 comprising the following steps:
a. cultivating a host cell, said host cell comprising a nucleic acid encoding said polypeptide, and expressing the nucleic acid under suitable conditions; and
b. isolating the polypeptide with suitable means.

36. (canceled)

37. (canceled)

38. (canceled)

39. (canceled)

40. (canceled)

41. (canceled)

42. An enzyme additive for consumer products, such as food products, selected from the polypeptide of claim 1, a nucleic acid enclosing said polypeptide, a vector comprising said nucleic acid, a host cell comprising said vector, functional fragments and functional derivatives thereof.

43. An additive for consumer products, such as food products, and/or particularly as a food additive for the degradation of fats, selected from the polypeptide of claim 25, a nucleic acid enclosing said polypeptide, a vector comprising said nucleic acid, a host cell comprising said vector, functional fragments and functional derivatives thereof.

44. A method for treating paper pulp comprising contacting said pulp with the polypeptide of claim 1 or a nucleic acid encoding said polypeptide, functional fragments, or functional derivatives thereof.

45. A method for producing an enzyme additive for use in consumer products, such as food products, comprising causing the production of the polypeptide of claim 1 by a material selected from a nucleic acid encoding said polypeptide, a vector comprising said nucleic acid, and a host cell comprising said vector.

46. A method for producing an enzyme for use in consumer products, such as food products, and/or particularly as a food additive for the degradation of fats, comprising causing the production of the polypeptide of claim 25 by a material selected from a nucleic acid encoding said polypeptide, a vector comprising said nucleic acid, and a host cell comprising said vector.

47. An enzyme for the pulp and paper industry, or for treating starting material for the production of paper, selected from the polypeptide of claim 1, a nucleic acid enclosing said polypeptide, a vector comprising said nucleic acid, a host cell comprising said vector, functional fragments and functional derivatives thereof.

48. An enzyme for the pulp and paper industry, or for treating starting material for the production of paper, selected from the polypeptide of claim 25, a nucleic acid enclosing said polypeptide, a vector comprising said nucleic acid, a host cell comprising said vector, functional fragments and functional derivatives thereof.

49. A method for producing an enzyme for the pulp and paper industry, or for treating starting material for the production of paper, comprising causing the production of the polypeptide of claim 1 by a material selected from a nucleic acid encoding said polypeptide, a vector comprising said nucleic acid, and a host cell comprising said vector.

50. A method for producing an enzyme for the pulp and paper industry, or for treating starting material for the production of paper, comprising causing the production of the polypeptide of claim 25 by a material selected from a nucleic acid encoding said polypeptide, a vector comprising said nucleic acid, and a host cell comprising said vector.

51. An enzyme for the preparation of nutritional lipids, selected from the polypeptide of claim 25, a nucleic acid enclosing said polypeptide, a vector comprising said nucleic acid, a host cell comprising said vector, functional fragments and functional derivatives thereof.

52. A method for producing an enzyme for the preparation of nutritional lipids, comprising causing the production of the polypeptide of claim 25 by a material selected from a nucleic acid encoding said polypeptide, a vector comprising said nucleic acid, and a host cell comprising said vector.

53. An enzyme for oleochemistry, in particular, for the manufacture of oleochemicals, selected from the polypeptide of claim 25, a nucleic acid enclosing said polypeptide, a vector comprising said nucleic acid, a host cell comprising said vector, functional fragments and functional derivatives thereof.

54. A method for producing an enzyme for oleochemistry, in particular, for the manufacture of oleochemicals, comprising causing the production of the polypeptide of claim 25 by a material selected from a nucleic acid encoding said polypeptide, a vector comprising said nucleic acid, and a host cell comprising said vector.

55. A method for the preparation of a medicament in the treatment of digestive disorders and/or diseases of the pancreas, comprising causing the production of the polypeptide of claim 25 by a material selected from a nucleic acid encoding said polypeptide, a vector comprising said nucleic acid, and a host cell comprising said vector.

56. A medicament in the treatment of digestive disorders and/or diseases of the pancreas, selected from the group consisting of the polypeptide of claim 25, a nucleic acid encoding said polypeptide, a vector comprising said nucleic acid, and a host cell comprising said vector.

57. An enzyme for treating textiles or fabrics, and/or e.g. as an ingredient in a detergent composition or fabric softener, said enzyme or ingredient selected from the group consisting of the polypeptide of claim 25, a nucleic acid enclosing said polypeptide, a vector comprising said nucleic acid, a cell comprising said vector, functional fragments and functional derivatives thereof.

58. A method for producing an enzyme for treating textiles or fabrics, and/or e.g. as an ingredient in a detergent composition or fabric softener, comprising causing the production of the polypeptide of claim 25 by a material selected from a nucleic acid encoding said polypeptide, a vector comprising said nucleic acid, and a host cell comprising said vector.


SEQUENCE LISTING (44 sequences)

SEQ ID NO: 1 - DNA, 807 bp, Unknown
  Description of Unknown Sequence CDS of clone pBKRR 09 obtained from the genomic library constructed from ruminal ecosytem (figure 10)
atgaaacgaaaaatatgctacatcctcatcgctctgaccaccacactggcggttgcagcacagaccacactcacccacccctggacaggaaaacgtgtggctttctttggcgactccatcacagaccccaagaacgctgcttcacaaaacaaatactggagcctgttgcaagaatggcttcacatcaccccatacgtctatgccgtcagcggcagacaatggaacgatattcctcgacagactgagcagctgcagacggaacacggacaggacgtcgatgcgattctcatcttcatcggcaccaatgacttcaatgcaggagtacccatcggtcaatggttcatcgaacaggaggaagaagtgatggcaggcatccatgaacccaagcacctggtaaaacggttgcgtaggattcccgatatgaatgccgacacctatcgtgggcgcatcaacatcgccttagacaaggtgaaacgcacctatcctgacaagcagatcgtgctgctcacacccatccatcgtgcaggattctatcgcaatgagaaaaactggcagcccacagaagactatcagaacaagtgtggcgagtggatatcagcgtatgtcgagtctgtgaaagaagcaggcaacctctgggccatgcccgttatcgacctcaacgccctctgtggcctctaccccctaatggacgaatacgtaccttatttcaatgatggcgccgttgaccgtctgcaccccaacgacaaaggccacgagcgtatggctcgcaccctctattaccagttgtctgcgctgcctgtatattag

SEQ ID NO: 2 - DNA, 810 bp, Unknown
  Description of Unknown Sequence Identical CDS of clone pBKRR 13 and clone pBKRR 52 obtained from the genomic library constructed from ruminal ecosytem (figure 11)
atgaaacaacgaatatatacatttatcctgacggctctgctatgcggagccgtcaatgctcaaacaggtttcacacatccttggatgcagaagcgtgtagcctttttcggtgactccattactgatccgaagcacaaaggatccaacgtaaaatactggcaatacctacaagattggttaggtattaccccctatgtatatggcataagcggacgccagtggaatgatattcccaaccaaaccgacaagttacagaaagagcatggcgacacggttgatgccattcttatttttataggcactaacgatttcaatgacggagtacccattggcgagtggtatgacatcaaggaagaaaaggtgatggctggtatccatgaacccaagcacatggtggttcgcaaacgcatgtatccaatcatgacgacaaccacgttcaaaggccgcattaatatagcgctcgataaattaaagcgtgcttatccaaccaagcaggtagtattgctcacacctatacaccgtgcaggattctatgcgaacgacaagaattggcaaccaacagaagactattacaatcgctgtggtgaaccctttaaacgttatatagacgccgtcaaagaggctggtaacatatgggcagtacctgttattgacttgaattccctaagtggccttttccctatgatggatgaacatgtcgtgtacttcaaggacggtaaaaacgaccgtctgcatcctaatgaaaaaggacacgagcgtatagcccgtaccctttattaccaacttaatgccctaccctgctgtttctaa

SEQ ID NO: 3 - DNA, 579 bp, Unknown
  Description of Unknown Sequence CDS of clone pBKRR 14 obtained from the genomic library constructed from ruminal ecosytem (figure 12)
atggccatcatagtgaagagcgctttacaccagatggacacggagtcgtacgacaaggccatggctgacaagaccgacaagcgtacaacggagattcctgccggcatagtatttccgcgtgaggttgctcaatataccatcggaaccatgcaggtatttgaagtgcctgctgagaacgaagagaaacctgtagtactctatctgcatggcggcgcttacgtccataactttacgtctcagcactggaaggccatggctgagtgggcaaaggccacaggctgtggcatcgtggctcccaactacccgttgctgcctctccatactgctgctgaggctcacctgctcatgatgcagctttatcgtgaactgctgaagggtattcctgcccaccgtattcttatcatgggcgactctgcaggtggcggctttacgctggcgctggcacagcagatacgcaatgactcgctcgacctgcctcgccatctggtgcttatctcgccctgggttgatgtcatgggcggtgattcttcgctgcaggagcgcgacaactggctcaccattgatgtgttgcagaaannn

SEQ ID NO: 4 - DNA, 1887 bp, Unknown
  Description of Unknown Sequence CDS of clone pBKRR 17 obtained from the genomic library constructed from ruminal ecosytem (figure 13)
gatccaaatttctacatcttcctctgtttcggacagtcgaacatggaaggcaatgcacgtcctgaggctgtggatcttgaaagtcctggaccccgattccttttaatgcccgcagtggacttccccgacaagggtcgtaagatgggcgaatggtgtgaggcttcggctcccctctgtcgtcctaacacaggtttgacccccgccgactggttcggtcgtaccttggtggcttcactgcccgagaacatcaagatcggtgtaatccacgtggctgtaggtggaatcaagatcgagggtttcatgcccagcgagattgccaactatgtgaagacggaagctccaggttggatgaagggaatgctggaggcatacggtaacaacccctatgaacgattggttactttggccaagaaggcacagaaggatggtgtaatcaagggtatcctgatgcaccagggcgagtcgaataccggtgatcccgactgggccaagaaggtgcagaaggtgtatgattccctttgcagcgacctgaagctgaagcccgaggatgtgcccctcttcgctggtaatatcgtacaggccaacgggcagggtgtttgcattggttgcaagaagcagatcgacgagctgccgcagaccattcataccagtcaggtgatctcctcagacgactgctctaacggtcctgaccgtctgcacttcgatgctgcaggctaccgtgagctgggttgccgctatggcgaggccgttgcccgtttcctgggcttcgagcccaagcgtcctaagatgccgggtaagaagattgttgttcctgccgatgcaaagattgccgagaccaccgttcccggtaacgactttccgaagatcgactcacagcgtcgcggctatttctatctgagtgctcctgatgcacagaaggtagtgcttgatatctgcgacaagaaatacgacatgcagtcggacggcaaaggtggctggatggctgttactgatccgctggtggaaggcttccactattatttcatgaatatcggtggtgttaacttcatcgatcctgctacagagaccttcttcggctgtaaccgtgaggctggtggcttcgaggttcctgaaggacccgaaggcgactactatcgtccgcagcagggtattgagcatggaaaggtgagcagcatctactacttcagtaatgagcagcagacatggcgccacgccatggtctatacccccgctggctatgatgccaagaagaacatcaagaagcgctacccggtgctctatctgcagcatggtatgggtgaggatgagaccggctggagcaagcagggacacatgcagcacatcatggacaacgccattgccagtggcgaggctgttcctatgattgtggtgatggagagtggtgacatcaaggccccgatgggtcgtggacaaggtatggacagctatggtaacacgttctatcctgttctcttgaacgacctgattccgtatatcgatgccaactaccgtaccaagaccgatcgtgacaaccgtgccatggccggactttcttggggtggtcatcaaaccttcgacattgtgttcaacaacctcgacaagttctcttacctgggaaccttcagcggtgccatcttcaacctcgacgtgaagacagcctacgatggtgtgttcaccaaggctgacgagctgaacaagaagattcactacttctttatgatgagcggtaccgagggaatggacaagatgttcggaaccgaacgactagtgaagagtctgaacgatctgggggtgaatgctcattactatgagtcaacaggtactgcccatgagtggcttacttggcgccgcggtctgaagcagttcattccgcatctgtttaaataa

SEQ ID NO: 5 - DNA, 774 bp, Unknown
  Description of Unknown Sequence CDS of clone pBKRR 27 obtained from the genomic library constructed from ruminal ecosytem (figure 14)
atgatggaagtatggctgaccaaatcgggtggatggagcatcacaaacaacacaccgattagcggaatcatcagcattgaatactccacagcgaaatctgcgtcttacagcggaaaaacactgtcgatcttgggcgattcaatcagcacctatgctggctacattccaagcggacagtcggcattctatgacggtacaaactgcggtgtatcttcggttgaccagacatggtggaaacggattatcaactcgctcgacatgacactgaatcttaataactcatggggcggtggaagagtttccaagacacgaagcacctatacagaagaatcttcgggtatctaccgtgcggataaactcggcacagatcccgatgtgattatcacctaccttggaatcaacgatttcaacggtgaggtatccaaagcggtattcaaatcatcctacgaaaccatgctcgataacatgaagacggcatacccgaacgcagaaatcttctgcgctacgttaccgccttgcgaacgaaatggtagtacaggcgatcccgaaatcaatgatgatggagtggcattgactgagtacaacgatatcattcgtgaagtcatcttggaaaagggagtgaaactgcttgactttgcaaactgcggaattacctacaacaccctgtcgcagtacatgggagactgggagagcgcaacaggcagagcattacatcctaattcggaaggacacagactgattgctcagaaggctatcagggatatgtttgattaa

SEQ ID NO: 6 - DNA, 3231 bp, Unknown
  Description of Unknown Sequence CDS of clone pBKRR 34 obtained from the genomic library constructed from ruminal ecosytem (figure 15)
gatcctgtggagcccaagtgggtgaaggatacagcccttctcacagaggactcgcagattacctccaacaactcccaggacggcttcccgcccagcaatctgttgcgccccgagagtgaaggctacgctaccaaccaaatcatctggcacagtgcatggacaccggccgctccggcaggcaccgaaacctacctgcagacccacttcgcctcagcccagcagcatatcatcttcaccatgataggctccatgtgggcgagcacctacgacacccccaccgagatagtgctctacgccaccaatgacccctccggcgagtggactgagataacgacgctcactgagatgtctgccgatttcaccagtttctcgcccgatatgtatgagtcgccccacatcgacctcggcgctgagtacaccgacctccgcttcgtggtgaagaagaccatgaccgaaagctccgctgtccgtcatgatgccaacggcaacccttacgtctcactcggccgtttccaagtgtacagtgccaaggaggcaggcgatgaccccatcgaccccaaggacaatatcaacctgctgttcataggcaacagtatcacttacggcgccaccctcggctcacctgccagccaggcaccccccatcctgtgccgcgcgatgatacaggaggccaccggcgtcacgaccaacgtctataacggcggccattcaggcatcaccactctgggcttcctgccgggacgcaccgacttcatcatggtactcaatagcgcccgcaccttcgttaagcaaaacggaggcctcacctacttctccatcatgctgggcaccaacgacagcgcatgcagcggccccgagggctcgcccgtgtcacctgccacctatgccgccaatatccgcaagataatcaacgccctcatcgaggcaataccctcgtgcaagatagtgctcaactatcctatctggtatagccccaacactcacaacggcgccgtctatctgcaggaaggcctcgaccgcttgcacagctactatcccgtcatcgacgaggtggtggaggagtacgaccaggtgtatgccggcgaccgcggcgtttgggaatatttcgaggataacaagacgctctttactgacgagcccggcaattcgggcaacttctgcctccaccccaaccagtacggggccaagcgcctcgccgagatatggtcgcgcagcctccttaagattatcgaggctgacggtgttgagataaagaatcccatcgccgactggccggagtttaagcccgccgccgacaagaaatacaccatctccaccccgcgcggcacttatggcaccaaagacggcctcctcaccaacaccgtgcgccagggcatcggtgccaccgagggcgagtttgccttcatcacctacgagggccagacctacctctatagcgttgccgatcagtcgttcctgttccgcgaccctgtgccctatcaggacaactggagcaacatggtactctccaaccagagcttcgtgcccatcaaggtcaactacaccggcatcagcagcgcctatccctataccattgcttctgagggctacatagccaacaccgccaacaacacccagaccggtgtatgcttcaacacctatatcagccctaacgacggcggcaaccagacagccattaccgaggcaggcgactttgatacctccgaggcgtatgccatgctcgagaacttcttcaccaatcaggtggaagtgctctattgcgtcgttgattcagacggcaacgccctcgactccgtctacctcgctggcgcagcaggtaccctcatcgaccaggcgccctctgcactgccccgcaaggcctacaccgactactccgtgcccgagcctgtcaccctgcagaaggagggcgacaacgtggtcaatgtgcttgccacatggcgcctgccctttgagctatcacccgaccgcgacaacgcgcattggtacaacctcgccctgcgtgagggctgcgactacgtgaccactaataacgcctacaagtgcaaccccgatgccactaaggaagacctggagagcacagcctaccagtgggcgtttgacggcaacccctacgagggcattgtcgtctattgtcgtgtcaaccctgccatgacgctcacccgtgtgaacaacaaggccttcctgcgcaacggcattttccgttggcagataatagagagcacacagggcttcctgctcgccaccgacgacaagacctatccctacatgaatgagtacggcggcgcaggcggcagtctcggcttctggaacaatatcagcgacgtgggcagcatcttcagcgtctgcgaagtcggcgtgcccaatgtcagcaacatcaagctctccacgggcggcagcctcaagatattcagggcacctgatgacaaggccaacggccgcgctatcctcgtctttccgggcggcggctacggtttcatagcaggccctaacgaaggcagcgactgggcacccatgttcaacaacctcgggtacaccgtgggcgtgcttacctacaccgtacccccatcgtcgcccgaccagcccctcactcaggcacgcgcagccatgtcatatctgcgcagccatagcgacgaatggaatgtcaataccggcatcattggcgtgataggcttcagcgccggcggccatctcgctgctaccgtagccacccataccagcggtggcgaagccccggcttttcagatactcttctatcccgtcatcaccatggacgccagctacacccactccggctcgcgccagaacctcataggcgacaaccccactcttgagcttgagactctatacagcaacgagaagcaagtcacctccaccacgccgcccgcctacatctgctgggcagacaacgacggcaccgtgccgcccgccaatagtatcaactatgccagcgccctcactgaaaagggtgtgcctgtgcgcacccgcaactatcccagcggcggccacggctacggctacggcatcgcttcgggatgggagtaccacgatgacatggtggcagacctcaccgcatggctcctcggcctcgaggacgacctcaccgccgtcaacagcatcccccgcgcatcagccatcaaggccccggcctactataacctatatggtcagcgggtgagtgagccgcgccagggtatatatatcaccgaagggaaaaagatcaggatcaagtaa

SEQ ID NO: 7 - DNA, 1566 bp, Unknown
  Description of Unknown Sequence CDS of clone pBKRR 35 obtained from the genomic library constructed from ruminal ecosytem (figure 16)
atgcctgcagtagactatcctgctacagataaactgccagcccgtaagatgggtgagtggtgcgaggctattcctccactctgtcgtcctaataccggactgactcctgccgactggttcggacgtactctcgtggcttcactgccggagaatatcaagataggtgttatccacgtagctatcggcggtatcgatatccgcggattcctcccagatagtattccttcttatgttaagagggctcccaactggatgaaaggcatgctcgaggcatataacaataatccttacgagcgattggtgactttagccaagaaagctcagaaggatggtgtcatcaagggcatcctgatgcatcagggtgagacaaacactggtgacccgaagtgggcaggaatggtgcagcaggtgtatgatcatctctgtggcgacctgcagctgaagccagaggatgtgaacctctatgcgggaaatatcgtccaggctggtggccagggtgtctgctttgcttgtaagaaacagatcgacgaactcccccagaccctgcatacagcacaggtgatctcttctgatgactgtagcaacggtcctgatcgtctgcatttcgatgcggcaggctatcgtgagctgggttgccgttatggtgaggctgtagcacgattcctcggctatgagcctaagcgtccttatatcgagatgccaaagaagatagaagttccagaggatgcctttatcgcagaaacaaccgttcccggtaatgagtttccgaaggtggacaaggagggacgtgcatatttccgtatccaggctcctgaggcccgtaaggtggtgctcgacatctgcagcaagaaatatgatatgcagtctgacggaaagggtggctggatggctgttaccgatcctctcgtacgcgggttccattattattttatgaatatcggaggcgtgaacttcattgatcctgccacagagacattctttggatgtaaccgtgaggcaggtggtatcgagattcctgagggtgctgagggtgactactaccgtccacagcaaggcgtggctacgggtgaggtgcgcagcttctactactatgcagagtcgacaaaggagtggcgtcacgctatggtttacacccctgcagagtatgacctgaagaaaaatgccaagaaacgctatcctgtgctctatctgcagcacggtatgggtgaggacgagacaggctggagcaagcagggtcacatgcagcatatcatggacaatgccatcgccagcggtaaggctgtgccaatgattgttgttatggagagtggtgacatcaaggctcctttcagaggcggcgacaaccgtcagggcatgagcacctacggcaactcgttctacaaagttataatcaacgatttaataccaaccatagaccagaagttccgcaccctgacagatcgtgaccatcgtgcgatggcagactgtcttggggcggacaccaaaccttcgacatcgtgctcaacaacatggataagttctcgtatatcggaacattcagcggagcaatcttcggactcgatgtga

SEQ ID NO: 8 - DNA, 1992 bp, Unknown
  Description of Unknown Sequence CDS of clone pBKRR 40 obtained from the genomic library constructed from ruminal ecosytem (figure 17)
atgcaaacgaatacggcgcgatattcttcccgggcagaacgcgacgtgtaccataccgacgacgtggacggcggcaaaaagatatatgaatatccacattactcaacgttctttcagacggactacgccattatcggaaagtcaacgcgggactggatgctctatccgattacgcgccttgagatcgagccgccggagccggtaattgaccttgtggatgttcccggctccgattatgcccttgacctcacggaatccctgacgggcagaccgatatacaagcaacgcgaatgcgattgggtgtttatcattgtcgcgccgcggaatcagtgggacgcgatatacagcgacgtgatgaaccgcctgcacggacggcgaatgaaggtcgttagaatggaagagccggactattactatgagggcagaataacgattgagtccacaaaatcggacaagtggaacgggcatatcaagatgcacggagtttttgacccgtataagagaaacgttcttgcatcggacgacgactggctgtgggacccgttctcgttcgaggatggctatatcccgtatcagccgaatatatacgcaatcggtcacagcctgcccgaaacatacaagagcatcgtcgtatcaacggatagccctacgtcaattctgttcggcgaaacgtttatgccaacatcaccgacgattaacgtattgaagccgacagatagcgttatgacgctgcaaattgagggcgaagaggaaacggtaacgctgaacgacggcgataacagaatccccggtattctcggcagaccggaacgcgcctgttaccctgacgttctcactgacaaattcaacggcatccggcaacagcgcaaaagtagcgttaaaattccggggaggttcgctgtaatgtattcgatttatataaataacgaagacaacaccggcgaacatctcctttattcaccgacaaacgttgacgaaggcgcgattgtgcttgaaccgaagctcaagatggaaataaacaaggcgatgacccttgaatttcttttgccgccgacaaatccgctttacggagatattgcaaagctgaaaacaacgataacgctgtacgacggttcgaccctcaaatttcgcgggcggtgcagggagacaaaaaaatcatttaacaaatgcgtcgaatatacctgctcgtccgaattgtcttttctcggagatgttgacactgaaccgtataacaacggcgattctgacaacactaaaaagaccgtcggcgggtggatcgcattttttctcgaaaaatacaacgcgcaggcgaagacggcgcggaagattcagccggggaacacaacggacggcggctccactacattcaagatgagcaacgacggcgattcgacggtgtttgatgagattatggcaattgccgaagcgcgaaacggaatcattgagacgcgcagagagggcggcacaacataccttgatttccgaacagtaaaagaagctgaccgctcaacgcagattattgaattcggcaagaatctgatcgactttgaacagtttgtcgatgcgacggagatcgttacgcacgttaaggcttacgacaaggacaatgcgcacagcgttacggcgacaaacacaaccgcagaaaccacatacggaacgcgggtacaccgtgttatgcggtgggatatgatcgaggatcaaaccacactgcaatctatggcaaatagctatgtgaacgcggcttatgcgatggcggcgacgatcactcttgaagcggttgaccttcatttgattaaagcagacgaacagcaattccggctcggatatgagaatcgaatgctttcaccgccgcacggcgtggacgaatggtttttgtgttcgcggatcgaacttgatttgacaaagcccgcaaaaaacaagtacgtcttcggcgcgacacgtgcaacgctaaccgaaaaagtcacatacaaaccgacagtattgagggggtaa

SEQ ID NO: 9 - DNA, 668 bp, Unknown
  Description of Unknown Sequence CDS of clone pBKRR 41 obtained from the genomic library constructed from ruminal ecosytem (figure 18)
nnggctttgtcgcgaaggattccccggcgcaggctgcgggcattttgccaggcgatacgattaccgccatgaacgacaaggcgacccagggctgggacgatttccgcgaacagattggcgtgagtctcggcgcggaagttcccctcacggtgcatcgcggaggcaagcccatcactgtcaccgtcgttcccgaagaactcgtgattcccgcacaagattccaccggttccgaaattaagatgggtatcggcgatatcggtatttacccgcagaaccgcgtgatggtgcgccttccgcccgtggcgggctccgcggcagaaaaagcgggcatcctcgaaaacgatacgatttttgaaatcaacggggagcatatctcccgctacgaagacgtggtgcgcattattgacggcagcaagggcgaaccggtgaacattaccgtcattcgcgaaggcgatacgttgaccaagacgcttagcgctatctataacgaggaacacaagcgctacatggtcggtatccagatgggctacgtgctcttccgcgaaacgaaactggtgcgtcgtggccccgtggaggcgtttaccaagacgtgcgccaccagctggaaaaatgacgacgagtatcttccgctacttcaagcgcatgttccaaggccaggtgaaggttga

SEQ ID NO: 10 - DNA, 951 bp, Unknown
  Description of Unknown Sequence CDS of clone pBKRR 43 obtained from the genomic library constructed from ruminal ecosytem (figure 19)
atgaacaggacaacaaagaagattctgcgaaccgtgacgggcatcatagttgccctcgttattattggtggggctgtatggatgattaccggtctctcacctcaggcacttatcgtgagggctttcctgaaacccatgaccatggataaatatgaggaagccatgaacgacaagaccgataagagtacgacggaaatacctgccgaagtgaagttcccccgcgaagtggctgaatacagggttggcggtatgcaggtgtttgaagtgcctgctgctgacgacaccaagcccgtggtgctctatctgcatggtggcgcttatgtacacaacttcaccccccagcattggaaggcgatggccgaatgggccaaggcaacgggctgcggcatcgtcgcccccaactatccgctgctgcccctgcatacagctgccgaggcccatcctatggtgatgcagctctatcgcgaactgctgaagggcatcgcttcacatcgcattcttatcatgggcgactctgcaggaggaggtttcacgttggcgctggctcagagacttgtggctgattcgctcgacctcccaagtcatctggtgctgatctcaccctgggttgacgtgatgggtggcgacccttccatacaagagcatgacaattggcttaccgtcgatgtgttacaaaaatatggcgctgattgggctgacggcatcgatgtcaacgacccgatgatctcaccccttaatggtgatatgaacggactaccgcccaccgacctttttaccggtacgtgggaggtgttttacaccgatgttctcaaaacctacgagaagatgaaggctgctggagttaaagtccgactgcatgttgctgagaagatgggccacgtttatccgctccatcccagtcctgagggtcgtaaagcgcgcaaggagatagcagatatcatcagaaaatag

SEQ ID NO: 11 - DNA, 849 bp, Unknown
  Description of Unknown Sequence CDS of clone pBKRR 44 obtained from the genomic library constructed from ruminal ecosytem (figure 20)
atggaaggccagggcgttatcgaagattgcgacctttctcccgatgaacgcttcctgatgatgtcaacgctcgactgcggaacccgcaagctcggacaatggtatcgcgccatcccgccgctggcacgttgtgacacccatctctgccctgccgattattttggccgcaccatggtagcgaaccttgacgaaggcaagcgcgtgggtgttgtggtggtagccatcggcggcattaatattgatttgtacgatcccgacggctggcagtcgtatgtgggaacaatgaacgagagttggcagatcaatgctgtcaacgcttacggcggcaatcccctggggcgtttgcttgaatgtgccagggaagcacagaagagcggtgtcatcaaaggtatcctcctgcatcagggggaaaacgatgcctacagcagcgtgtggctgcagaaagtgaaaaaggtgtatgagaacctactcgcggaactcaaccttaatgccgaagacgtgcctctcattgctggcgaggtaggcaatgaagaccaaaacggtatttgttgcgccgccaacaacaccatcaaccgcttgccccagaccattcctacggcacacgtcgtgtcttccgtaggttgtaccctgcagagtgacaacttgcacttcgattcaaagggctaccgcaaactgggacgccgctatgccaagaccatgcttgccacaatgggaatcgaagcagatatcgacgaggacgaggtgccaccaatcgattactctcagcccattgacataaccaaccgcttcacttattgctggaacaatgccgaaaccattacctctgtacggcggcatacttgtctataa

SEQ ID NO: 12 - DNA, 1980 bp, Unknown
  Description of Unknown Sequence Identical CDS of clone pBKRR 45 and clone pBKRR 48 obtained from the genomic library constructed from ruminal ecosytem (figure 21)
atgaacaagagacaaacgctactatgggtgtcggctatggcctgcgcctttgagatgaatgccaaggtgacactgccgcaactgttccaggacggcatggtgctgcaacgtgaaaagaccattcccgtctggggcaaggccgatgctggcgaggcggtcagcgtgacgctgaataagaagacctgccagacgacggcgacggccgacggccgatggcgggtcgacttgccgaagatgaaagccggcgggccatatatcttaacggtcaacgacgtcgagctgaaggatgtgttcatcggcgacgtatggctgctgtcgggccagtcgaacatcgacgtgaccgtcgagcgcgtctatccgtggtacacgacggacatcgacaactacaagaatcccaagatccgcctgttccgcgtgcagaacgagaccgacacccacggcgtgcgcgacgacatacgccccacgaccatcaactggaagcccgtcaaccgcgagaacgcctggctgttctctgcaatgggttattttctgggacgccggatgtacgagaagacccacgtggcacagggcatcattgtcaacagctggggcggcacacccattgaggcgtggctcagcgccgactccctcaatcagcattacccgatgctcgtcgaaaagacacgtctttaccagaacgatgactacgtgcgcacccagcagcgcgccaacatgctgatgagccagcagtggaacaaattgctcgaagagcgcgaccctggcaaaaagaccgatttcaccgctatcgactataacgattcaaaatggacgaaagttaaccaatattcgatggaatgggccaaaaaaggtaatcgtggcatcatcggcacgatctggctgcgccagcatgtgaccatcgacaaagaccactccggcaaacccgcgcgactgttgctcggtacgctgtttgactccgacgtaacctacctgaatggcaagcagataggtacaacgggctatcagtatccgccgcgccgctacgacattccagaaggactgctccgcgagggcgacaacgtcatcaccgttcgctttatcaataagtatggtacggcgcactttattccagagaagccctacctcatcgcctttggcgacgaccgcaagagcatgaaccccatgccgaaggacgtcgtgcccctgagcgagacgtggctccatcatgccggtgccgagatgctgtcgtgccccagcgccgacgtcagcctgcaaaacctcccaacgacgctctacaacgccgtgctctacccgttagcgccgtacgccctgtcaggcgtggtgtggtatcagggagagtcgaacacgggcaacccccgcccctacgagcactatctgacaatgctcgtcacgggctggcgccagctgtggcagcaacccgacctgccctttaccatcgtgcagctggccaatcatgacggtcgccagcagacgggcaacccgtcgccgctcacgccacagatagagccgcagcccaactccggctgggcacagctacgtgaggctcagcgcctggtagccaagaagctggacaacgtggagctggcttcggccatcgacctcggcgagcctgtcgacatccacccgctgcgtaagcgtgaggtggccgagcgcatcggactctgcttcgaccgtaccgtctatcatgacaagaaagtgaaactgatgcccgagattgtcggcaccaacatcgacggccgcaccgtcacgctgacctttgaccagccgctgcgtcccaatttagcgctctgtgagtttgaagtggctggcagcgacggacacttctccaatgctgcagcacgcgccgtgggcaacaccgtcatcatcgactcgcccatcgacaaccccgtccgcgtgcgccatgcatggaaagacaaccccatccagctgaatgcctacagccagacggggttgccaattggaccttttgagatttcgctctaa

SEQ ID NO: 13 - protein, 268 aa, Unknown
  Description of Unknown Sequence Amino acid of clone pBKRR 09 obtained from the genomic library constructed from ruminal ecosytem (figure 22)
MKRKICYILIALTTTLAVAAQTTLTHPWTGKRVAFFGDSITDPKNAASQNKYWSLLQEWLHITPYVYAVSGRQWNDIPRQTEQLQTEHGQDVDAILIFIGTNDFNAGVPIGQWFIEQEEEVMAGIHEPKHLVKRLRRIPDMNADTYRGRINIALDKVKRTYPDKQIVLLTPIHRAGFYRNEKNWQPTEDYQNKCGEWISAYVESVKEAGNLWAMPVIDLNALCGLYPLMDEYVPYFNDGAVDRLHPNDKGHERMARTLYYQLSALPVY

SEQ ID NO: 14 - protein, 269 aa, Unknown
  Description of Unknown Sequence Identical amino acid of clone pBKRR 13 and clone pBKRR 52 obtained from the genomic library constructed from ruminal ecosytem  (figure 23)
MKQRIYTFILTALLCGAVNAQTGFTHPWMQKRVAFFGDSITDPKHKGSNVKYWQYLQDWLGITPYVYGISGRQWNDIPNQTDKLQKEHGDTVDAILIFIGTNDFNDGVPIGEWYDIKEEKVMAGIHEPKHMVVRKRMYPIMTTTTFKGRINIALDKLKRAYPTKQVVLLTPIHRAGFYANDKNWQPTEDYYNRCGEPFKRYIDAVKEAGNIWAVPVIDLNSLSGLFPMMDEHVVYFKDGKNDRLHPNEKGHERIARTLYYQLNALPCCF

SEQ ID NO: 15 - protein, 195 aa, Unknown
  Description of Unknown Sequence Amino acid of clone pBKRR 14 obtained from the genomic library constructed from ruminal ecosytem (figure 24)
MAIIVKSALHQMDTESYDKAMADKTDKRTTEIPAGIVFPREVAQYTIGTMQVFEVPAENEEKPVVLYLHGGAYVHNFTSQHWKAMAEWAKATGCGIVAPNYPLLPLHTAAEAHLLMMQLYRELLKGIPAHRILIMGDSAGGGFTLALAQQIRNDSLDLPRHLVLISPWVDVMGGDSSLQERDNWLTIDVLQKXXX

SEQ ID NO: 16 - protein, 628 aa, Unknown
  Description of Unknown Sequence Polypeptide of clone pBKRR 17 obtained from the genomic library constructed from ruminal ecosytem (figure 25)
DPNFYIFLCFGQSNMEGNARPEAVDLESPGPRFLLMPAVDFPDKGRKMGEWCEASAPLCRPNTGLTPADWFGRTLVASLPENIKIGVIHVAVGGIKIEGFMPSEIANYVKTEAPGWMKGMLEAYGNNPYERLVTLAKKAQKDGVIKGILMHQGESNTGDPDWAKKVQKVYDSLCSDLKLKPEDVPLFAGNIVQANGQGVCIGCKKQIDELPQTIHTSQVISSDDCSNGPDRLHFDAAGYRELGCRYGEAVARFLGFEPKRPKMPGKKIVVPADAKIAETTVPGNDFPKIDSQRRGYFYLSAPDAQKVVLDICDKKYDMQSDGKGGWMAVTDPLVEGFHYYFMNIGGVNFIDPATETFFGCNREAGGFEVPEGPEGDYYRPQQGIEHGKVSSIYYFSNEQQTWRHAMVYTPAGYDAKKNIKKRYPVLYLQHGMGEDETGWSKQGHMQHIMDNAIASGEAVPMIVVMESGDIKAPMGRGQGMDSYGNTFYPVLLNDLIPYIDANYRTKTDRDNRAMAGLSWGGHQTFDIVFNNLDKFSYLGTFSGAIFNLDVKTAYDGVFTKADELNKKIHYFFMMSGTEGMDKMFGTERLVKSLNDLGVNAHYYESTGTAHEWLTWRRGLKQFIPHLFK

SEQ ID NO: 17 - protein, 260 aa, Unknown
  Description of Unknown Sequence Mature peptide of clone pBKRR 17 obtained from the genomic library constructed from ruminal ecosytem (figure 25)
VPEGPEGDYYRPQQGIEHGKVSSIYYFSNEQQTWRHAMVYTPAGYDAKKNIKKRYPVLYLQHGMGEDETGWSKQGHMQHIMDNAIASGEAVPMIVVMESGDIKAPMGRGQGMDSYGNTFYPVLLNDLIPYIDANYRTKTDRDNRAMAGLSWGGHQTFDIVFNNLDKFSYLGTFSGAIFNLDVKTAYDGVFTKADELNKKIHYFFMMSGTEGMDKMFGTERLVKSLNDLGVNAHYYESTGTAHEWLTWRRGLKQFIPHLFK

SEQ ID NO: 18 - protein, 257 aa, Unknown
  Description of Unknown Sequence Amino acid of clone pBKRR 27 obtained from the genomic library constructed from ruminal ecosytem (figure 26)
MMEVWLTKSGGWSITNNTPISGIISIEYSTAKSASYSGKTLSILGDSISTYAGYIPSGQSAFYDGTNCGVSSVDQTWWKRIINSLDMTLNLNNSWGGGRVSKTRSTYTEESSGIYRADKLGTDPDVIITYLGINDFNGEVSKAVFKSSYETMLDNMKTAYPNAEIFCATLPPCERNGSTGDPEINDDGVALTEYNDIIREVILEKGVKLLDFANCGITYNTLSQYMGDWESATGRALHPNSEGHRLIAQKAIRDMFD

SEQ ID NO: 19 - protein, 1076 aa, Unknown
  Description of Unknown SequencePolypeptide of clone pBKRR 34 obtained from the genomic library constructed from ruminal ecosytem (figure 27)
DPVEPKWVKDTALLTEDSQITSNNSQDGFPPSNLLRPESEGYATNQIIWHSAWTPAAPAGTETYLQTHFASAQQHIIFTMIGSMWASTYDTPTEIVLYATNDPSGEWTEITTLTEMSADFTSFSPDMYESPHIDLGAEYTDLRFVVKKTMTESSAVRHDANGNPYVSLGRFQVYSAKEAGDDPIDPKDNINLLFIGNSITYGATLGSPASQAPPILCRAMIQEATGVTTNVYNGGHSGITTLGFLPGRTDFIMVLNSARTFVKQNGGLTYFSIMLGTNDSACSGPEGSPVSPATYAANIRKIINALIEAIPSCKIVLNYPIWYSPNTHNGAVYLQEGLDRLHSYYPVIDEVVEEYDQVYAGDRGVWEYFEDNKTLFTDEPGNSGNFCLHPNQYGAKRLAEIWSRSLLKIIEADGVEIKNPIADWPEFKPAADKKYTISTPRGTYGTKDGLLTNTVRQGIGATEGEFAFITYEGQTYLYSVADQSFLFRDPVPYQDNWSNMVLSNQSFVPIKVNYTGISSAYPYTIASEGYIANTANNTQTGVCFNTYISPNDGGNQTAITEAGDFDTSEAYAMLENFFTNQVEVLYCVVDSDGNALDSVYLAGAAGTLIDQAPSALPRKAYTDYSVPEPVTLQKEGDNVVNVLATWRLPFELSPDRDNAHWYNLALREGCDYVTTNNAYKCNPDATKEDLESTAYQWAFDGNPYEGIVVYCRVNPAMTLTRVNNKAFLRNGIFRWQIIESTQGFLLATDDKTYPYMNEYGGAGGSLGFWNNISDVGSIFSVCEVGVPNVSNIKLSTGGSLKIFRAPDDKANGRAILVFPGGGYGFIAGPNEGSDWAPMFNNLGYTVGVLTYTVPPSSPDQPLTQARAAMSYLRSHSDEWNVNTGIIGVIGFSAGGHLAATVATHTSGGEAPAFQILFYPVITMDASYTHSGSRQNLIGDNPTLELETLYSNEKQVTSTTPPAYICWADNDGTVPPANSINYASALTEKGVPVRTRNYPSGGHGYGYGIASGWEYHDDMVADLTAWLLGLEDDLTAVNSIPRASAIKAPAYYNLYGQRVSEPRQGIYITEGKKIRIK

SEQ ID NO: 20 - protein, 239 aa, Unknown
  Description of Unknown Sequence Mature peptide of clone pBKRR 34 obtained from the genomic library constructed from ruminal ecosytem (figure 27)
MFNNLGYTVGVLTYTVPPSSPDQPLTQARAAMSYLRSHSDEWNVNTGIIGVIGFSAGGHLAATVATHTSGGEAPAFQILFYPVITMDASYTHSGSRQNLIGDNPTLELETLYSNEKQVTSTTPPAYICWADNDGTVPPANSINYASALTEKGVPVRTRNYPSGGHGYGYGIASGWEYHDDMVADLTAWLLGLEDDLTAVNSIPRASAIKAPAYYNLYGQRVSEPRQGIYITEGKKIRIK

SEQ ID NO: 21 - protein, 521 aa, Unknown
  Description of Unknown Sequence Amino acid of clone pBKRR 35 (figure 28)
MPAVDYPATDKLPARKMGEWCEAIPPLCRPNTGLTPADWFGRTLVASLPENIKIGVIHVAIGGIDIRGFLPDSIPSYVKRAPNWMKGMLEAYNNNPYERLVTLAKKAQKDGVIKGILMHQGETNTGDPKWAGMVQQVYDHLCGDLQLKPEDVNLYAGNIVQAGGQGVCFACKKQIDELPQTLHTAQVISSDDCSNGPDRLHFDAAGYRELGCRYGEAVARFLGYEPKRPYIEMPKKIEVPEDAFIAETTVPGNEFPKVDKEGRAYFRIQAPEARKVVLDICSKKYDMQSDGKGGWMAVTDPLVRGFHYYFMNIGGVNFIDPATETFFGCNREAGGIEIPEGAEGDYYRPQQGVATGEVRSFYYYAESTKEWRHAMVYTPAEYDLKKNAKKRYPVLYLQHGMGEDETGWSKQGHMQHIMDNAIASGKAVPMIVVMESGDIKAPFRGGDNRQGMSTYGNSFYKVIINDLIPTIDQKFRTLTDRDHRAMADCLGADTKPSTSCSTTWISSRISEHSAEQSSDSM

SEQ ID NO: 22 - protein, 663 aa, Unknown
  Description of Unknown Sequence Amino acid of clone pBKRR 40 obtained from the genomic library constructed from ruminal ecosytem (figure 29)
MQTNTARYSSRAERDVYHTDDVDGGKKIYEYPHYSTFFQTDYAIIGKSTRDWMLYPITRLEIEPPEPVIDLVDVPGSDYALDLTESLTGRPIYKQRECDWVFIIVAPRNQWDAIYSDVMNRLHGRRMKVVRMEEPDYYYEGRITIESTKSDKWNGHIKMHGVFDPYKRNVLASDDDWLWDPFSFEDGYIPYQPNIYAIGHSLPETYKSIVVSTDSPTSILFGETFMPTSPTINVLKPTDSVMTLQIEGEEETVTLNDGDNRIPGILGRPERACYPDVLTDKFNGIRQQRKSSVKIPGRFAVMYSIYINNEDNTGEHLLYSPTNVDEGAIVLEPKLKMEINKAMTLEFLLPPTNPLYGDIAKLKTTITLYDGSTLKFRGRCRETKKSFNKCVEYTCSSELSFLGDVDTEPYNNGDSDNTKKTVGGWIAFFLEKYNAQAKTARKIQPGNTTDGGSTTFKMSNDGDSTVFDEIMAIAEARNGIIETRREGGTTYLDFRTVKEADRSTQIIEFGKNLIDFEQFVDATEIVTHVKAYDKDNAHSVTATNTTAETTYGTRVHRVMRWDMIEDQTTLQSMANSYVNAAYAMAATITLEAVDLHLIKADEQQFRLGYENRMLSPPHGVDEWFLCSRIELDLTKPAKNKYVFGATRATLTEKVTYKPTVLRG

SEQ ID NO: 23 - protein, 223 aa, Unknown
  Description of Unknown Sequence Amino acid of clone pBKRR 41 obtained from the genomic library constructed from ruminal ecosytem (figure 30)
XXGFVAKDSPAQAAGILPGDTITAMNDKATQGWDDFREQIGVSLGAEVPLTVHRGGKPITVTVVPEELVIPAQDSTGSEIKMGIGDIGIYPQNRVMVRLPPVAGSAAEKAGILENDTIFEINGEHISRYEDVVRIIDGSKGEPVNITVIREGDTLTKTLSAIYNEEHKRYMVGIQMGYVLFRETKLVRRGPVEAFTKTCATSWKNDDEYLPLLQAHVPRPGEG

SEQ ID NO: 24 - protein, 316 aa, Unknown
  Description of Unknown Sequence Amino acid of clone pBKRR 43 obtained from the genomic library constructed from ruminal ecosytem (figure 31)
MNRTTKKILRTVTGIIVALVIIGGAVWMITGLSPQALIVRAFLKPMTMDKYEEAMNDKTDKSTTEIPAEVKFPREVAEYRVGGMQVFEVPAADDTKPVVLYLHGGAYVHNFTPQHWKAMAEWAKATGCGIVAPNYPLLPLHTAAEAHPMVMQLYRELLKGIASHRILIMGDSAGGGFTLALAQRLVADSLDLPSHLVLISPWVDVMGGDPSIQEHDNWLTVDVLQKYGADWADGIDVNDPMISPLNGDMNGLPPTDLFTGTWEVFYTDVLKTYEKMKAAGVKVRLHVAEKMGHVYPLHPSPEGRKARKEIADIIRK

SEQ ID NO: 25 - protein, 282 aa, Unknown
  Description of Unknown Sequence Amino acid of clone pBKRR 44 obtained from the genomic library constructed from ruminal ecosytem (figure 32)
MEGQGVIEDCDLSPDERFLMMSTLDCGTRKLGQWYRAIPPLARCDTHLCPADYFGRTMVANLDEGKRVGVVVVAIGGINIDLYDPDGWQSYVGTMNESWQINAVNAYGGNPLGRLLECAREAQKSGVIKGILLHQGENDAYSSVWLQKVKKVYENLLAELNLNAEDVPLIAGEVGNEDQNGICCAANNTINRLPQTIPTAHVVSSVGCTLQSDNLHFDSKGYRKLGRRYAKTMLATMGIEADIDEDEVPPIDYSQPIDITNRFTYCWNNAETITSVRRHTCL

SEQ ID NO: 26 - protein, 659 aa, Unknown
  Description of Unknown Sequence Identical amino acid of clone pBKRR 45 and clone pBKRR 48 obtained from the genomic library constructed from ruminal ecosytem (figure 33)
MNKRQTLLWVSAMACAFEMNAKVTLPQLFQDGMVLQREKTIPVWGKADAGEAVSVTLNKKTCQTTATADGRWRVDLPKMKAGGPYILTVNDVELKDVFIGDVWLLSGQSNIDVTVERVYPWYTTDIDNYKNPKIRLFRVQNETDTHGVRDDIRPTTINWKPVNRENAWLFSAMGYFLGRRMYEKTHVAQGIIVNSWGGTPIEAWLSADSLNQHYPMLVEKTRLYQNDDYVRTQQRANMLMSQQWNKLLEERDPGKKTDFTAIDYNDSKWTKVNQYSMEWAKKGNRGIIGTIWLRQHVTIDKDHSGKPARLLLGTLFDSDVTYLNGKQIGTTGYQYPPRRYDIPEGLLREGDNVITVRFINKYGTAHFIPEKPYLIAFGDDRKSMNPMPKDVVPLSETWLHHAGAEMLSCPSADVSLQNLPTTLYNAVLYPLAPYALSGVVWYQGESNTGNPRPYEHYLTMLVTGWRQLWQQPDLPFTIVQLANHDGRQQTGNPSPLTPQIEPQPNSGWAQLREAQRLVAKKLDNVELASAIDLGEPVDIHPLRKREVAERIGLCFDRTVYHDKKVKLMPEIVGTNIDGRTVTLTFDQPLRPNLALCEFEVAGSDGHFSNAAARAVGNTVIIDSPIDNPVRVRHAWKDNPIQLNAYSQTGLPIGPFEISL

SEQ ID NO: 27 - DNA, 966 bp, Unknown
  Description of Unknown Sequence R.34 nucleotide sequence (figure 34)
atgaatgagtacggcggcgcaggcggcagtctcggcttctggaacaatatcagcgacgtgggcagcatcttcagcgtctgcgaagtcggcgtgcccaatgtcagcaacatcaagctctccacgggcggcagcctcaagatattcagggcacctgatgacaaggccaacggccgcgctatcctcgtctttccgggcggcggctacggtttcatagcaggccctaacgaaggcagcgactgggcacccatgttcaacaacctcgggtacaccgtgggcgtgcttacctacaccgtacccccatcgtcgcccgaccagcccctcactcaggcacgcgcagccatgtcatatctgcgcagccatagcgacgaatggaatgtcaataccggcatcattggcgtgataggcttcagcgccggcggccatctcgctgctaccgtagccacccataccagcggtggcgaagccccggcttttcagatactcttctatcccgtcatcaccatggacgccagctacacccactccggctcgcgccagaacctcataggcgacaaccccactcttgagcttgagactctatacagcaacgagaagcaagtcacctccaccacgccgcccgcctacatctgctgggcagacaacgacggcaccgtgccgcccgccaatagtatcaactatgccagcgccctcactgaaaagggtgtgcctgtgcgcacccgcaactatcccagcggcggccacggctacggctacggcatcgcttcgggatgggagtaccacgatgacatggtggcagacctcaccgcatggctcctcggcctcgaggacgacctcaccgccgtcaacagcatcccccgcgcatcagccatcaaggccccggcctactataacctatatggtcagcgggtgagtgagccgcgccagggtatatatatcaccgaagggaaaaagatcaggatcaagtaa

SEQ ID NO: 28 - DNA, 966 bp, Unknown
  Description of Unknown Sequence EL1 nucleotide sequence (figure 35)
atgaatgagtacggcggcgcaggcggcagtctcggcttctggaacaatatcagcgacgtgggcagcatcttcagcgtctgcgaagtcggcgtgcccgatgtcagcaacatcaagctctccacgggcggcagcctcaagatattcagggcacctgatgacaaggccaacggccgcgctatcctcgtctttccgggcggcggctacggtttcatagcaggccctaacgaaggcagcgactgggcacccatgttcaacaacctcgggtacaccgtgggcgtgcttacctacaccgtacccccatcgtcgcccgaccagcccctcactcaggcacgcgcagccatgtcatatctgcgcagccatagcgacgaatggaatgtcaataccggcatcattggcgtgataggcttcagcgccggcggccatctcgctgctaccgtagccacccataccagcggtggcgaagccccggcttttcagatactcttctatcccgtcatcaccatggacgccagctacacccactccggctcgcgccagaacctcataggcgacaaccccactcttgagcttgagactctatacagcaacgagaagcaagtcacctccaccacgccgcccgcctacatctgctgggcagacaacgacggcaccgtgccgcccgccaatagtatcaactatgccagcgccctcactgaaaagggtgtgcctgtgcgcacccgcaactatcccagcggcggccacggctacggctacggcatcgcttcgggatgggagtaccacgatgacatggtggcagacctcaccgcatggctcctcggcctcgaggacgacctcaccgccgtcaacagcatcccccgcgcatcagccatcaaggccccggcctactataacctatatggtcagcgggtgagtgagccgcgccagggtatatatatcaccgaagggaaaaagatcaggatcaagtaa

SEQ ID NO: 29 - protein, 321 aa, Unknown
  Description of Unknown Sequence R.34 amino acid sequence (figure 36)
MNEYGGAGGSLGFWNNISDVGSIFSVCEVGVPNVSNIKLSTGGSLKIFRAPDDKANGRAILVFPGGGYGFIAGPNEGSDWAPMFNNLGYTVGVLTYTVPPSSPDQPLTQARAAMSYLRSHSDEWNVNTGIIGVIGFSAGGHLAATVATHTSGGEAPAFQILFYPVITMDASYTHSGSRQNLIGDNPTLELETLYSNEKQVTSTTPPAYICWADNDGTVPPANSINYASALTEKGVPVRTRNYPSGGHGYGYGIASGWEYHDDMVADLTAWLLGLEDDLTAVNSIPRASAIKAPAYYNLYGQRVSEPRQGIYITEGKKIRIK

SEQ ID NO: 30 - protein, 321 aa, Unknown
  Description of Unknown Sequence EL1 amino acid sequence (figure 37)
MNEYGGAGGSLGFWNNISDVGSIFSVCEVGVPDVSNIKLSTGGSLKIFRAPDDKANGRAILVFPGGGYGFIAGPNEGSDWAPMFNNLGYTVGVLTYTVPPSSPDQPLTQARAAMSYLRSHSDEWNVNTGIIGVIGFSAGGHLAATVATHTSGGEAPAFQILFYPVITMDASYTHSGSRQNLIGDNPTLELETLYSNEKQVTSTTPPAYICWADNDGTVPPANSINYASALTEKGVPVRTRNYPSGGHGYGYGIASGWEYHDDMVADLTAWLLGLEDDLTAVNSIPRASAIKAPAYYNLYGQRVSEPRQGIYITEGKKIRIK

SEQ ID NO: 31 - protein, 314 aa, Butyrivibrio fibrisolvens
  MISC_FEATURE: description of sequence amino acid sequence from B.fib = beta-1,4-D-xylanase (Figure 44)
KGKCEAKEAYYAVLKAAVSDDSIDKWVPDYSEEDYKLQGMPTPDIKRFRENIWQENEYNYEASYGFIPNLFAYLHNDDVKRDCMLVIPGGGYCMCCSHEGELAAMEFYNRGMNAFVLSYTTDITMSVPLHKQPLEDISRAVRFIRKNASKYNIDGKKLVIMGFSAGSHVCGSLAVHFDDVKDNNPEYADISGRPDGVILSYPVITTGRYTHADSVRTLLGANPTDEELTYFSLEKQVKDNTPPCFIWQTEEDSVVPVENSYLFANALREKKIPFAHYVFPRGFHGLTVANDEFFSGWSGGEYSMEQTMRARFAV

SEQ ID NO: 32 - protein, 272 aa, Chlostridium acetobutylicum ATCC 824 (Figure 44)
  misc_feature: description of sequence  amino acid sequence of C. ace = acetyl esterase family enzyme fom Clostridium acetobutylicum (Figure 44)
MINRIIDIWEGFSYKSSDNSDFRPKMETYILNGDKKRSAILILPGGGYNHTSSREAEPIAVNYNAAGFSTFVLHYSVAPNRYPQPLLDAARAISIIRENADEWNIDKDKIAVCGFSAGGHLAGLLGVNWFKEELFNVDGISKEFLKPNALILCYPVITSGKFAHKKSFKCLLGDEGKKLLDEVSIEKHVTGKTPTTFIWHTFNDKVVPVQNSMLFTEALSKVEVSFEMHIYPEGKHGMALGTKETDGGGNINPHLASWFNLSVEWIRSIFGE

SEQ ID NO: 33 - protein, 310 aa, A. acidocaldarius
  misc_feature: description of sequence amino acid sequence of EST2 = esterase from A. acidocaldarius (PDB accession number 1EVQA; crystal structure) (see Figure 44)
MPLDPVIQQVLDQLNRMPAPDYKHLSAQQFRSQQSLFPPVKKEPVAEVREFDXDLPGRTLKVRXYRPEGVEPPYPALVYYHGGGWVVGDLETHDPVCRVLAKDGRAVVFSVDYRLAPEHKFPAAVEDAYDALQWIAERAADFHLDPARIAVGGDSAGGNLAAVTSILAKERGGPALAFQLLIYPSTGYDPAHPPASIEENAEGYLLTGGXXLWFRDQYLNSLEELTHPWFSPVLYPDLSGLPPAYIATAQYDPLRDVGKLYAEALNKAGVKVEIENFEDLIHGFAQFYSLSPGATKALVRIAEKLRDALA

SEQ ID NO: 34 - DNA, 19 bp, Artificial
  Description of artificial sequence  OligF sense Primer (description p. 59)
cctatccctataccattgc

SEQ ID NO: 35 - DNA, 21 bp, Artificial
  Description of artificial sequence  OligR antisense Primer (description p. 59)
ccgtccatataatacttcagg

SEQ ID NO: 36 - DNA, 21 bp, Artificial
  Description of artificial sequence  Mut34FpCR sense Primer (description p. 59)
cctatccctataccattgctt

SEQ ID NO: 37 - DNA, 49 bp, Artificial
  Description of artificial sequence  Mut34RpCR antisense Primer (description p. 59)
tttagtggtggtggtggtggtgcttgatcctgatctttttcccttcggt

SEQ ID NO: 38 - DNA, 33 bp, Artificial
  Description of artificial sequence  R49D Primer for mutagenesis (description p. 62)
gcctcaagatattcgacgcacctgatgacaagg

SEQ ID NO: 39 - DNA, 33 bp, Artificial
  Description of artificial sequence  R49D Primer for mutagenesis (description p. 62)
ccttgtcatcaggtgcgtcgaatatcttgaggc

SEQ ID NO: 40 - DNA, 33 bp, Artificial
  Description of artificial sequence  R49N Primer for mutagenesis (description p. 62)
gcctcaagatattcaacgcacctgatgacaagg

SEQ ID NO: 41 - DNA, 33 bp, Artificial
  Description of artificial sequence  R49N Primer for mutagenesis (description p. 62)
ccttgtcatcaggtgcgttgaatatcttgaggc

SEQ ID NO: 42 - DNA, 21 bp, Artificial
  Description of artificial sequence  F1 Primer for sequencing (description p. 62)
aacaacaaggccttcctgcgc

SEQ ID NO: 43 - DNA, 21 bp, Artificial
  Description of artificial sequence  F2 Primer for sequencing (description p. 62)
tgggcgtgcttacctacaccg

SEQ ID NO: 44 - DNA, 21 bp, Artificial
  Description of artificial sequence  F3 Primer for sequencing (description p. 62)
acatctgctgggcagacaacg